(19) United States
(12) Patent Application Publication
(10) Pub. No.: US 2026/0152760 A1
(43) Pub. Date: Jun. 4, 2026

(54) Binary Self-Amplifying Nucleic Acid Platform and Uses Thereof

(71) Applicants: The University of British Columbia, Vancouver (CA); The James Hutton Institute, Dundee (GB)

(72) Inventors: Wilfred Arthur JEFFERIES, Vancover (CA); Kyung-Bok CHOI, Vancover (CA); Paolo RIBECA, Edinburgh (GB)

(21) Appl. No.: 19/126,757

(22) PCT Filed: Oct. 31, 2023
(86) PCT No.: PCT/CA2023/051444

§ 371 Date: May 2, 2025

Related U.S. Application Data
(60) Provisional application No. 63/422,549, filed on Nov. 4, 2022.
(60) Provisional application No. 63/429,633, filed on Dec. 2, 2022.
(60) Provisional application No. 63/537,906, filed on Sep. 12, 2023.

(51) Int. Cl. C12N 15/85; A61K 39/00; A61K 39/215; A61P 37/04; C07K 14/005

(52) U.S. Cl. CPC C12N 15/85; A61K 39/215; A61P 37/04; C07K 14/005; A61K 2039/53; C12N 2770/20034; C12N 2770/36122; C12N 2820/60

(57) ABSTRACT

The present invention relates to an expression vector that encodes all or a portion of replicon proteins from a positive stranded virus, wherein expression of the replicon proteins is under the control of CMV and T7 promoters, and wherein expression of a pay load is under the control of a sub-genomic promoter. Also provided are methods of using the vector in therapeutics and vaccines.

### FIELD OF THE INVENTION

This invention generally pertains to a binary self-amplifying nucleic acid platform, and uses thereof.

### BACKGROUND OF THE INVENTION

The zoonotic transmission of pathogens from animals to humans has become the kindling of emergent diseases, with the frequency dramatically increasing in our shift from hunters and gatherers to agrarian societies (1). Human interactions with animals through hunting, animal farming, trade of animal-based foods, wet markets or exotic pet trade (2), together with increased human interactions through global trade and travel (3) have ignited the fires of global pandemics. In the 20^{th} century alone, five major pandemics emerged, including, Smallpox, HIV/AIDS (1976), the sixth cholera pandemic (1899, 1923), the Spanish flu (1918 to 1920), and the Swine flu (2009), that resulted in over 100 Million deaths world-wide (4). Yet in the 21^{st} century, despite the sparks created by SARS-CoV-1 (2003), the global zeitgeist remained in the dark and was unprepared for the bonfire that became SARS-CoV-2 (2019). If not for the promethean intervention of the biotechnology community, combined with truly herculean efforts of public health authorities to collectively quell the flame of the COVID-19 pandemic through the swift introduction of first-generation SARS-CoV-2 vaccines, humanity would likely have been reduced to ashes, but may yet become a mere ember, in the absence of better, more effective vaccines (5). The first-generation vaccines were developed under an unprecedented, accelerated scheme underpinned by breakneck production and delivery rollout, with only a few months elapsing from design to testing and approval, and by in large, target the spike protein of SARS-CoV-2. The initial SARS-CoV-2 vaccines to appear on the world stage mainly differ in their underlying delivery platforms: Oxford/Astra Zeneca vaccine is based on a recombinant adenovirus vector, while Moderna and Pfizer vaccines are both based on a mRNA-based platform. Additional vaccine designs based on various formulation principles have since been developed in a number of countries, which include Novavax, a protein-based design containing spike and Valneva, a vaccine containing the inactivated virus (6). mRNA delivery systems have offered the advantage of rapid development of vaccines (7-9). This platform has been shown to be safe, effective, and adaptable for a variety of therapeutic applications (7-9). However, mRNA systems have been limited by their requirement for highly technical manufacturing, their inherent thermal instability (10) and their inefficient in vivo delivery in the absence of lipid nanoparticles (LNPs) (11). Overall, there remains a societal need to create new and more effective platforms with the clear aim of achieving sterilizing immunity.

Self-amplifying RNA (saRNA) delivery platforms stand out as leading technologies in vaccine development with the potential to solve many of the issues that have been highlighted for other platforms. Recombinant saRNA expression vectors featured an engineered replicon that can encode and drive high levels of antigen expression (12). Very low doses (micrograms) compared to mRNA technologies may only be required, as tens of thousands of copies of saRNAs are synthesized directing immense amounts of payload mRNA transcription within recipient cells (12), and furthermore, saRNA vaccines can be delivered relatively noninvasively by intramuscular injection, similar to mRNA or DNA vaccines (12). Self-amplifying vaccines are considered safe and capable of inducing humoral and cellular immunity and they can also avoid the induction of anti-vector immunity, while simultaneously reducing the risk of the vector genome integration into the host genome (12). Manufacturing advantages when compared with conventional vaccines include a lower intrinsic risk of contamination with live infectious reagents and a much better scalability when mass production is required. On the other hand, the current generation of saRNA vectors also shares a number of issues with conventional mRNA platforms: they require technically demanding production processes involving in vitro transcription; their stable during long-term storage is an open question, and conventional dogma suggests they rely upon costly and technically demanding LNP encapsulation to allow uptake into cells where they express their payload proteins (12).

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An objective of the present invention is to provide a binary self-amplifying nucleic acid platform and uses thereof.

In accordance with an aspect of the invention, there is provided a vector comprising: one or more promoters; replicon protein genes from Venezuelan equine encephalitis (VEE) virus under the control of the one or more promoters; a sub-genomic promoter from the VEE virus; a multi-cloning site for insertion of a nucleic acid encoding a payload under the control of the sub-genomic promoter or a nucleic acid encoding a payload under the control of the sub-genomic promoter and optionally resistance gene(s) for mammalian and/or bacterial cell culture.

In accordance with an aspect of the invention, there is provided a vector comprising: a CMV and T7 binary promoter; NSP1-4 replicon protein genes from Venezuelan equine encephalitis (VEE) virus under the control of the binary promoter; a 26S sub-genomic promoter from the VEE virus; a multi-cloning site for insertion of a nucleic acid encoding a payload under the control of the sub-genomic promoter or a nucleic acid encoding a payload under the control of the sub-genomic promoter or a nucleic acid encoding a payload under the control of the sub-genomic promoter and optionally resistance genes for puromycin (PuroR) and ampicillin (AmpR) for mammalian and bacterial cell culture, respectively.

In another aspect of the invention, there is provided a self-amplifying RNA vector comprising: mRNA encoding NSP1-4 replicon protein genes from Venezuelan equine encephalitis (VEE) virus and mRNA encoding a payload.

Also provided are pharmaceutical compositions comprising the vectors of the invention and a pharmaceutically acceptable carrier and methods of delivering a payload of interest or treating and/or preventing disease using the vectors of the invention.

### BRIEF DESCRIPTION OF THE DRAWINGS

These and other features of the invention will become more apparent in the following detailed description in which reference is made to the appended drawings.

FIG. 1. (A) Genomic map of the Gemini 1.0 vector. The vector contains a binary promoter (CMV and T7), replicon protein genes (NSP1-4), a 26S sub-genomic promoter from the Venezuelan equine encephalitis (VEE) virus, and resistance genes for puromycin (PuroR) and ampicillin (AmpR) for mammalian and bacterial cell culture, respectively. (B) Delivery mechanisms of Gemini as saDNA (Gemini-D) or saRNA (Gemini-R). In-vitro replication. Gemini-D (left) is delivered directly into the cell; it enters the nucleus for transcription then re-enters the cytoplasm for translation using the endogenous machinery to express its RNA dependent RNA polymerase (RdRp). Gemini-R (right) is delivered into the cytoplasm for direct translation of its RdRp by the endogenous machinery. The RdRp then transcribes the payload mRNA, allowing for production of the payload protein. saRNA is transcribed in vitro (top right) via the T7 promoter with the assistance of T7 polymerase. Also, 5′ capping is performed before mRNA delivery into the cytoplasm for direct translation of RdRp and subsequent expression of payload mRNA.

FIG. 2. Negative-strand RNA is present post-transfection with eGFP Gemini-R and Gemini-D. (A) Detection strategy: First-strand synthesis is conducted using a NSP4 region specific primer (purple) with a random nucleotide tag (red) to produce positive strand (+) cDNA. The negative (−) RNA strand is then synthesized as cDNA using primers specific for the eGFP region (green) and the random nucleotide tag, producing a band of about 1.9 kb. A subsequent nested PCR (primers: dark green and fuchsia) then produces a band of 1.4 kb. (B) Results of applying the strategy described in panel (A) to yield total RNA from HEK293 cells transfected with Gemini-R and Gemini-D expressing eGFP. Results of the first and nested PCRs are shown when the forward primer was used (columns 4 and 5) and not used (columns 2 and 3, control). The reference ladder in column 1 indicates the position and sizes of the molecular weight markers in base pairs (bp).

FIG. 3. Temporal dynamics of protein expression in HEK293 cells transfected with Gemini-R and Gemini-D expressing SARS-CoV-2 spike protein. LNP-encapsulated B.1.617.2 (Delta) spike protein variant of the SARS-CoV-2 virus expressed in saDNA (Gemini-D) or saRNA (Gemini-R) in HEK293 cells were compared with a conventional LNP-encapsulated plasmid DNA vector expressing the B. 1.617.2 (Delta) spike protein variant of the SARS-CoV-2 virus on day 2 and 6 using unpaired t-test. (A) Western Blot at day 6 after transfection (B) mRNA expression over time (C) Results of flow cytometry. Spike-expressing cells were subject to flow cytometric analysis on Day 2 and 6 post-transfection, using a BD Cytoflex flow cytometer (D) Quantitated fraction of positive cells (in percent's). p-values are indicated.

FIG. 4. Temporal dynamics of protein expression in HEK293 cells transfected with Gemini-R and Gemini-D expressing eGFP protein. Time course of LNP-encapsulated eGFP positive saDNA (Gemini-D) and saRNA (Gemini-R) cells were compared on weeks 2 and 4 after selection by cell sorting for eGFP positive. (A) Western Blot at day 6 after sorting (B) mRNA expression over time (C) HEK-293 cells were transfected with Gemini-D and Gemini-R expressing eGFP and the samples were sorted for GFP expression 2 days post-transfection. Sorted GFP-expressing cells were then subject to flow cytometric analysis on Day 2, then weekly up to 6 weeks for EGFP expression using a BD Cytoflex flow cytometer (D) Quantitated fraction of positive cells (in percent). eGFP positive saDNA and saRNA cells were compared on weeks 2 and 4 using unpaired t-test. p-values are indicated.

FIG. 5A-F. Non self-amplifying eGFP expression in vivo. Both Gemini-D (saDNA) and Gemini-R (saRNA) achieve long lasting expression of a protein payload in vivo. (A) Representative images showing eGFP fluorescence induced by Gemini-R and Gemini-D in mouse muscle tissue 14, 28 and 42 days after IM injection. (B) Quantitative fluorescence for Gemini-D estimated from selected images including those in panel (A) labelled saDNA. Day 42 is significantly higher than background (p<0.05). (C) Quantitative fluorescence for Gemini-R estimated from selected images including those in panel (A) labelled saRNA. Day 14 is significantly higher than background (p<0.05). Both non-serlf-amplifying vaccines, DNA and RNA, show lower levels of GFP expression in vivo compared to Gemini-D and Gemini-R respectively. (D) Representative images showing eGFP fluorescence induced by DNA and RNA in mouse muscle tissue 2, 6 and 14 days after IM injection. (E) Quantitative fluorescence for DNA estimated from selected images including those in panel (A) labelled DNA. (F) Quantitative fluorescence for RNA estimated from selected images including those in panel (A) labelled RNA. No values in E. or F. were significantly different from background.

FIG. 6. Thermal Stability of Gemini-D and Gemini-R and a conventional LNP-pseudouridine substituted mRNA vaccines all encoding spike protein from the B.1.617.2 (Delta) variant of the SARS-CoV-2 virus. Stability of Naked saDNA (A), Naked saRNA (B), and LNP-mRNA (C) was compared after many cycles of freezing and thawing (FT) at −80° C. and RT respectively. The stability of nucleic acids and lipid structures were examined. For the experiment, each vaccine sample (2 μg for Naked saDNA/mRNA and 1 μg for LNP-mRNA, respectively) was subject to freezing and thawing up to 5 times in total (sample number one is frozen and thawed 2×, 3×, 4× and 5×, respectively) and the gel images were documents using the gel doc system at our research facility.

FIG. 7. Effect of Freeze-Drying (FD) on different forms of vaccines. Assessment of the effect of saDNA, saRNA and LNP-mRNA after Freezing-Drying (FD; lyophilization).

FIG. 8. Immune response in mice inoculated with Gemini-D and Gemini-R expressing SARS-CoV-2 spike protein. A single dose of either the LNP-encapsulated Gemini-R and Gemini-D vaccine formats elicits significant antibody concentrations (A) Antibody concentration measured in mouse serum 28 days post-injection: Left: IgG response; Right: IgM response. All the treatment groups were statistically different than the unvaccinated control group (ANOVA, p=0.0082). (B) Comparison between immune response to Naked and encapsulated eGFP: Left: Gemini-D; Right: Gemini-R. All the treatment groups were statistically different than the unvaccinated control group (ANOVA, p=0.0134). See Materials and Methods for the meaning of bars and points. (C) Comparison between antibody response to Naked D-Gemini (100 μg), R-Gemini (25 μg) and LNP-encapsulated mRNA (5 μg) vaccine in mouse serum 28 days post-injection: IgG response. The antibody responses were also compared to the normal saline injected control (data not shown). Normal serum from age matched unvaccinated K18hAce2 transgenic mice were used as background control in the ELISA. Analysis of Variance (one way ANOVA) was conducted between all the groups including normal saline (data not shown). Multiple comparisons were done comparing normal saline to each group using Dunnett's post-hoc test. The p-value numbers are as follows: 0.0712 (saRNA Naked), 0.0082 (saDNA Naked), 0.0970 (mRNA LNP). (D) Comparison between antibody response to LNP-encapsulated D-Gemini (5 μg), R-Gemini (5 μg) and LNP-encapsulated mRNA (5 μg) vaccine in mouse serum 28 days post-injection: IgG response. The antibody responses were also compared to the normal saline injected control (data not shown). Normal serum from age matched unvaccinated K18hAce2 transgenic mice were used as background control in the ELISA. Analysis of Variance (one way ANOVA) was conducted between all the groups including normal saline (data not shown). Multiple comparisons were done comparing normal saline to each group using Dunnett's post-hoc test. The p-value numbers are as follows: 0.1675 (saRNA LNP), 0.0079 (saDNA LNP), 0.3757 (mRNA LNP). (E) Comparison between IFN-γ ELISPOT response to LNP-encapsulated D-Gemini, LNP-encapsulated R-Gemini, mRNA vaccine in mouse spleen cells post-harvest. Analysis of Variance (one way ANOVA) was conducted between all the groups including normal saline (data not shown). Multiple comparisons were done comparing normal saline to each group using Dunnett's post-hoc test. The p-value numbers are as follows: 0.0006 (LNP saDNA), 0.0370 (LNP saRNA), 0.0461 (LNP mRNA) (F) Comparison between IFN-γ ELISPOT response to Naked D-Gemini, Naked R-Gemini, mRNA vaccine in mouse spleen cells post-harvest. Analysis of Variance (one way ANOVA) was conducted between all the groups including normal saline (data not shown). Multiple comparisons were done comparing normal saline to each group using Dunnett's post-hoc test. The p-value numbers are as follows: 0.0050 (Naked saDNA), 0.3271 (Naked saRNA), 0.0461 (LNP mRNA). (G) Comparison between IL-4 ELISPOT response to LNP-encapsulated D-Gemini, LNP-encapsulated R-Gemini, mRNA vaccine in mouse spleen cells post-harvest. Analysis of Variance (one way ANOVA) was conducted between all the groups including normal saline (data not shown). Multiple comparisons were done comparing normal saline to each group using Dunnett's post-hoc test. The p value numbers are as follows: 0.9730 (LNP saDNA), 0.0004 (LNP saRNA), 0.0088 (LNP mRNA). (H) Comparison between IL-4 ELISPOT response to Naked D-Gemini, Naked R-Gemini, mRNA vaccine in mouse spleen cells post-harvest. Analysis of Variance (one way ANOVA) was conducted between all the groups including normal saline (data not shown). Multiple comparisons were done comparing normal saline to each group using Dunnett's post-hoc test. The p value numbers are as follows: 0.2048 (Naked saDNA), 0.0054 (Naked saRNA), 0.0088 (LNP mRNA). (I) Comparison between TNF-α ELISPOT response to LNP-encapsulated D-Gemini, LNP-encapsulated R-Gemini, mRNA vaccine in mouse spleen cells post-harvest. Analysis of Variance (one way ANOVA) was conducted between all the groups including normal saline (data not shown). Multiple comparisons were done comparing normal saline to each group using Dunnett's post-hoc test. The p values are as follows: 0.9906 (LNP saDNA), 0.0476 (LNP saRNA), 0.9813 (LNP mRNA). (J) Comparison between TNF-α ELISPOT response to Naked D-Gemini, Naked-R-Gemini, mRNA vaccine in mouse spleen cells post-harvest. Analysis of Variance (one way ANOVA) was conducted between all the groups including normal saline (data not shown). Multiple comparisons were done comparing normal saline to each group using Dunnett's post-hoc test. The p values are as follows: 0.9128 (Naked saDNA), 0.0011 (Naked saRNA), 0.9813 (LNP mRNA).

FIG. 9. Naked saDNA or saRNA Formats Result in Extended and Durable Antibody Responses

To determine if vaccination with either Naked saDNA or saRNA formats could result in extended and stronger antibody responses, a time-course study was conducted to evaluate this point. Age-matched K18-hACE2 female mice susceptible to SARS-Cov-2 were injected with nucleic acid vaccines through intramuscular (i.m.) route of injection on days 0. Mice were bled and serum collected on the indicated time points. IgG antibody levels to Omicron spike protein were assessed using the protocol described in the materials and methods. (A) Serum was collected from saRNA-Naked injected mice on days 28, 42 and 56. Analysis of Variance (one way ANOVA) was conducted between all the groups including normal saline (data not shown). Multiple comparisons were done comparing normal saline to each group using Dunnett's post-hoc test. None of the p values were significantly different than the normal saline control. (B) Serum was collected from saDRNA-Naked injected mice on days 28, 42 and 56 and 70. Analysis of Variance (one way ANOVA) was conducted between all the groups including normal saline (data not shown). Multiple comparisons were done comparing normal saline to each group using Dunnett's post-hoc test. The p-value numbers are as follows: 0.1882 (day 28), 0.0100 (day 42), 0.0075 (day 56), 0.0024 (day 70). 0.0609 (day 84).

FIG. 10. Immunization with SARS-CoV-2 Spike-specific Naked saDNA and LNP-saDNA reduces viraemia post-challenge. To conclusively establish whether Naked saDNA could outperform the LNP-based saDNA and potentially replace the need for LNP inclusion, a viral challenge experiment was carried out to directly compare the efficacy of LNP-encapsulated Omicron-Spike saDNA with that of Naked Omicron-Spike saDNA. K18-hACE2 mice susceptible to SARS-Cov-2 were injected with nucleic acid vaccines through intramuscular (i.m.) route of injection on days 0 and 28. Mice were challenged 5 days post-boost and sacrificed five days later. (A) Blood viraemia levels were assessed in sera collected from virus challenged mice, after receiving vaccinations of either encapsulated or Naked Omicron-Spike saDNA and challenged with the Omicron virus Nucleocapsid concentration in saDNA-LNP encapsulated vaccinated mouse blood viraemia samples collected 5 days post-challenge. The vaccinated group was significantly different than the unvaccinated control group using unpaired T test (p=0.0274). (B) Nucleocapsid concentration in saDNA-Naked vaccinated mouse blood viraemia samples collected 5 days post-challenge. The vaccinated group was significantly different than the unvaccinated control group using unpaired T test (p=0.0003)

### DETAILED DESCRIPTION OF THE INVENTION

The present inventors, as described in WO2022/246559 (incorporated by reference herewith), developed expression vectors that encode all or a portion of replicon proteins from a positive stranded virus, wherein expression of the replicon proteins is under the control of eukaryotic and prokaryotic promoters, and the expression of a payload is under the control of a sub-genomic promoter.

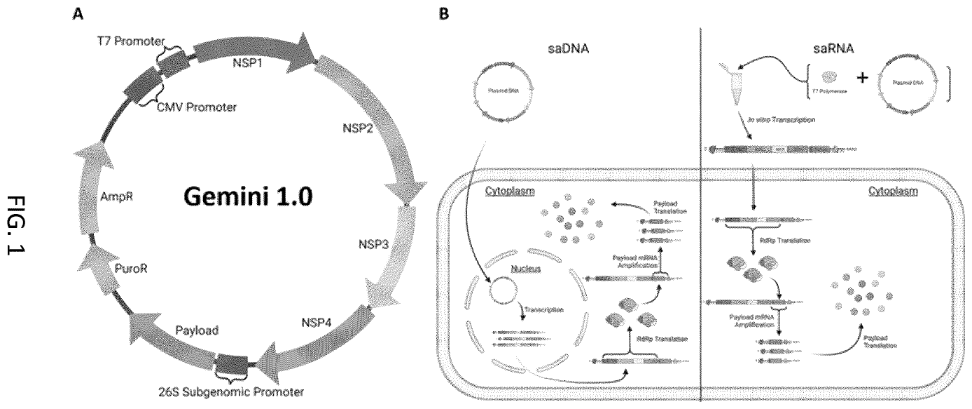

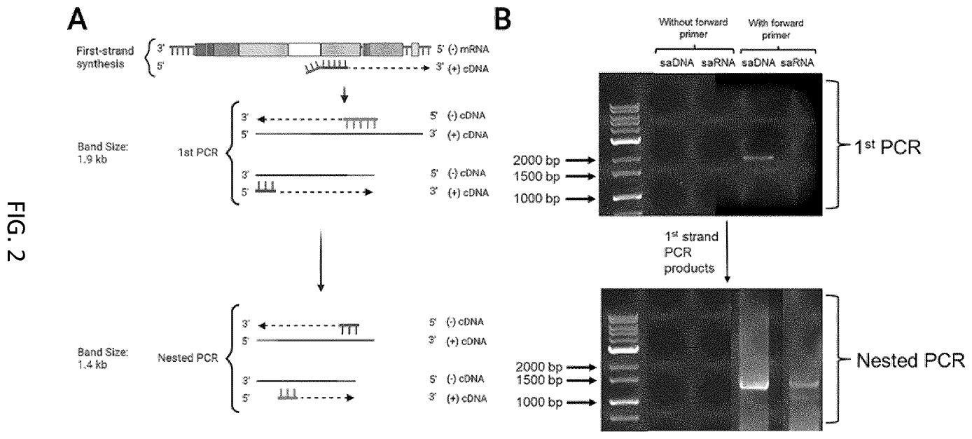

The present invention expands upon the inventor's previous work. The binary gene expression vectors of the present invention can function as either self-amplifying mRNA or self-amplifying DNA vectors; demonstrate stability after multiple freeze/thaw cycles and freeze-drying and do not require encapsulation with lipid nanoparticles (LNP) to achieve enduring gene expression in vivo. Accordingly, the present invention provides binary expression vectors which are stable after multiple freeze/thaw cycles and freeze-drying and/or do not require encapsulation with lipid nanoparticles (LNP). As detailed in FIG. 1B, the vectors of the present invention may be delivered to a cell in the form of a self-amplifying DNA vector or a self-amplifying RNA vector. In embodiments in which the self-amplifying DNA vector is utilized, the self-amplying DNA vector is delivered directly to the cell. In embodiments in which the self-amplifying RNA vector is for delivery to the cell, the self-amplying DNA vector is transcribed in vitro to produce the self-amplifying RNA vector which is then introduced to a cell.

In some embodiments, the present invention provides expression vectors based on positive stranded viruses belonging to the orders Nidovirales, Martellivirales and Hepelivirales and uses thereof. In particular in certain embodiments, the present invention provides a vector, including but not limited to a self-amplifying plasmid DNA vector, that encodes all or a portion of replicon proteins from a positive virus of interest and includes a multi-cloning site to allow insertion of a sequence of a payload of interest.

In some embodiments of the invention, the vector is a plasmid DNA vector encoding the replicon from a positive stranded virus where the expression of the replicon proteins is driven by a eukaryotic promoter and/or a prokaryotic promoter or a dual eukaryotic prokaryotic promoter. In some embodiments the promoter is a fused dual eukaryotic prokaryotic promoter. As used herein, the term promoter includes promoters and promoters plus enhancer elements. In specific embodiments, there is provided a vector comprising dual promoter CMV and T7.

The eukaryotic promoter may be constitutive, inducible or tissue specific. Exemplary eukaryotic promoters include but are not limited to CMV, EF1a, SV40, PGK1 (human or mouse), Ubc, human beta actin, CAG, TRE, UAS, Ac5, Polyhedrin, CaMKIIa, GAL1, 10, TEF1, GDS, ADH1, CaMV35S, Ubi, H1 and U6. Exemplary prokaryotic promoters include but are not limited to T7, T7lac, Sp6, araBAD, trp, lac, Ptac and pL.

In certain embodiments, the eukaryotic promoter is tissue specific. Exemplary tissue specific promoters include but are not limited to B29 promoter, CD14 promoter, CD43 promoter, CD45 promoter, CD68 promoter, Desmin promoter, Elastase-1 promoter, Endoglin promoter, Fibronectin promoter, Flt-1 promoter, GFAP promoter, GPIIb promoter, ICAM-2 promoter, mIFN-β promoter, Mb promoter, NphsI promoter, OG-2 promoter, SP-B promoter, SYN1 promoter, WASP promoter, SV40/bAlb promoter, SV40/hAlb promoter, SV40/CD43 promoter, SV40/CD45 promoter and NSE/RU5′ promoter.

In specific embodiments, the vector is a DNA plasmid driven by a CMV promoter with or without a T7 promoter. In such embodiments, once the plasmid enters the cell, the plasmid DNA will drive expression of the positive stranded RNA replicon that will in turn drive replication of the negative strand RNA that will begin the self-amplifying mRNA cycle. In other embodiments, the transcription takes place in vitro to produce the self-amplifying RNA vector which is then introduced to a cell.

In more specific embodiments, the vector is a self-amplifying plasmid DNA vector with dual promoter (such as a CMV and T7) which encodes all or a portion of the replicon proteins from the Venezuelan Equine Encephalitis (VEE) virus genome. In this embodiment, the dual promoter will drive transcription in vivo or in vitro of mRNA encoding all the replicon proteins necessary for self-amplification of mRNAs. One or more sub-genomic promoters will drive expression of downstream payloads.

The complete genome of VEE virus is known in the art and is published under GenBank Accession NC_001449. In certain embodiments of the invention, the vector includes the full viral replicon (i.e. the 5′ leader sequence, followed by the viral replicase gene), followed by the payload, followed by the viral 3′ terminal segment.

In specific embodiments, the vector comprises a promoter (including but not limited to a binary eukaryotic prokaryotic promoter), replicon protein genes from VEE virus under the control of the binary promoter, a sub-genomic promoter from the VEE virus, multi-cloning site for insertion of a nucleic acid encoding a payload under the control of the sub-genomic promoter and optionally resistance gene(s) for mammalian and bacterial cell culture, respectively. In more specific embodiments, the vector comprises a CMV and T7 binary promoter, NSP1-4 replicon protein genes from VEE virus under the control of the binary promoter, a 26S sub-genomic promoter from the VEE virus, multi-cloning site for insertion of a nucleic acid encoding a payload under the control of the sub-genomic promoter and optionally resistance genes for puromycin (PuroR) and ampicillin (AmpR) for mammalian and bacterial cell culture, respectively.

In specific embodiments, the vector comprises the sequence of any of the vectors set forth below. A worker skilled in the art would readily appreciate that the GFP and the Delta sequences are non-limiting exemplary payload sequences which may be replaced with other payload sequences.

### Payload

The vectors of the present invention may be utilized to express a variety of payloads, including one or more nucleic acids, one or more peptides and one or more polypeptides.

In certain embodiments, the payload is RNA, including but not limited to siRNA and shRNA. In certain embodiments, the payload is an aptamer. In certain embodiments, the payload is one or more polypeptides. The polypeptide(s) may be any polypeptide. Exemplary polypeptides including but not limited to immunogens; epitopes; antibodies, SFV; immunomodulatory molecules including but not limited to cytokines; growth factors; fusion proteins; suicideproteins; CRISPR CAS9 or other recombinase system and any other therapeutic proteins.

### Pharmaceutical Compositions

The present invention further comprises pharmaceutical compositions including vaccine formulations. The binary gene expression vectors of the present invention can function as either self-amplifying mRNA or self-amplifying DNA vectors. Accordingly, the present invention provides pharmaceutical compositions including vaccine formulations comprising either the self-amplifying mRNA or self-amplifying DNA vectors.

The vectors of the present invention demonstrate stability after multiple freeze/thaw cycles and freeze-drying. Accordingly, the vectors may be provided freeze-dried. In certain embodiments, the vectors of the present invention are provided as freeze-dried plasmid DNA nanomaterial.

The inventors of the present invention have found that encapsulation with lipid nanoparticles (LNP) is not required to achieve enduring gene expression in vivo. Accordingly in certain embodiments, the vectors are not encapsulated with LNPs. In specific embodiments, the vectors are provided as naked self-amplying DNA.

In alternative embodiments, the vectors are incorporated into liposomes, microspheres or other polymer matrices. Accordingly, in certain embodiments, the pharmaceutical compositions including vaccines formulations comprise lipid nanoparticle delivery formulations of vector. Optionally, the lipid is cationic. Appropriate cationic lipids are known in the art. Non-limiting examples include phosphatidylcholine/cholesterol/PEG-lipid, C12-200, dimethyldioctadecylammonium (DDA), 1,2-dioleoyl-3-trimethylammonium propane (DOTAP) or 1,2-dilinoleyloxy-3-dimethylaminopropane (DLinDMA). Also see for example, U.S. Pat. No. 10,221,127 (incorporated by reference) and Reichmuth A M et al. (Therapeutic Delivery. 2016; 7(5):319-334. DOI: 10.4155/tde-2016-0006). In specific embodiments, LNPs the comprise an ionizable cationic lipid (phosphatidylcholine: cholesterol/PEG-lipid (50:10:38.5:1.5 mol/mol). In certain embodiments, the vector to total lipid ratio in the LNP is approximately 0.05 (wt/wt). In certain embodiments, the LNPs have a diameter of ˜80 nm.

The pharmaceutical compositions including vaccines formulations optionally may comprise one or more pharmaceutically acceptable carriers, excipients and/or adjuvants. Adjuvants and carriers suitable for administering genetic vaccines and immunogens are known in the art. Conventional carriers and adjuvants are for example reviewed in Kiyono et al. 1996.

Exemplary adjuvants include mineral salts including but not limited to aluminium salts (such as amorphous aluminum hydroxyphosphate sulfate (AAHS), aluminum hydroxide, aluminum phosphate, potassium aluminum sulfate (Alum)) and calcium phosphate gels; Oil emulsions and surfactant based formulations, including but not limited to MF59, QS21 (purified saponin), AS02 [SBAS2] (oil-in-water emulsion+MPL+QS-21), Montanide ISA-51 and ISA-720 (immunoprec water-in-oil emulsion); Particulate adjuvants, including but not limited to virosomes (unilamellar liposomal vehicles incorporating influenza haemagglutinin), AS04 ([SBAS4] Al salt with MPL), ISCOMS (structured complex of saponins and lipids), polylactide co-glycolide (PLG). And; microbial derivatives (natural and synthetic), including but not limited to monophosphoryl lipid A (MPL), Detox (MPL+M. Phlei cell wall skeleton), AGP [RC-529] (synthetic acylated monosaccharide), DC_Chol (lipoidal immunostimulators able to self mmunopr into liposomes), OM-174 (lipid A derivative), CpG motifs (synthetic oligonucleotides containing immunostimulatory CpG motifs), modified LT and CT (genetically modified bacterial toxins to provide non-toxic adjuvant effects); endogenous human immunomodulators, including but not limited to hGM-CSF or hIL-12 (cytokines that can be administered either as protein or plasmid encoded), Immudaptin (C3d tandem array) and inert vehicles, such as gold particles.

The pharmaceutical compositions and vaccine formulations optionally may comprise a stabilizer. Suitable stabilizers are known in the art and include but are not limited to amino acids, antioxidants, cyclodextrins, proteins, sugars/sugar alcohols, and surfactants. See for example Morefield, AAPS J. 2011 June; 13(2): 191-200; https://www.ncbi.nlm.nih.gov/pmc/articles/PMC3085699/).

In certain embodiments, charge-altering releasable transporters (CARTs) are used to deliver the vectors.

In certain embodiments, the vector is formulated as a virus-like particle (VLP).

### Methods of Use

The present invention further provides a method of delivering a payload of interest to a cell, the method comprising contacting the cell (either in vitro or in vivo) with a vector of the present invention which expresses the payload. The cell may be a prokaryotic or eukaryotic cell. In certain embodiments, expression of the payload prevents, delays and/or treats disease.

As detailed in FIG. 1B, the vectors of the present invention may be delivered to a cell in the form of a self-amplifying DNA vector or a self-amplifying RNA vector. In embodiments in which the self-amplifying DNA vector is utilized, the self-amplying DNA vector is delivered directly to the cell. In embodiments in which the self-amplifying RNA vector is for delivery to the cell, the self-amplying DNA vector is transcribed in vitro to produce the self-amplifying RNA vector which is then introduced to a cell.

In some embodiments, the expression vectors of the present invention sustain prolonged expression of proteins, induce strong immune responses involving humoral antibodies and cell-mediated T lymphocytes against the target antigens.

The vector may be administered to a variety of subjects, including but not limited to prokaryotes and eukaryotes. In certain embodiments, the subject is a human or other animals, including but not limited to other mammals, such as non-human primates, cats, dogs, equines (including but not limited to horses, donkeys and zebras), camels, sheep, goats, and bovines (including but not limited to cows).

In certain embodiments, the vectors of the present invention are used as vaccines. In such embodiments, the vectors may comprise as a payload one or more sequences encoding one or more epitopes or antigens of interest. For example, a vector for use as a SARS-CoV-2 vaccine will include one or more sequences encoding one or more SARS-CoV-2 antigens or epitopes as a payload. Accordingly, also provided herein is a method of treating, protecting against, and/or preventing disease associated with the infectious agent in a subject in need thereof by administering the vaccine to the subject. For example, a worker skilled in the art would readily appreciate that a SARS-CoV-2 vaccine may be used treating, protecting against, and/or preventing disease associated with SARS-CoV-2 (i.e., COVID 19). Administration of the vaccine to the subject can induce or elicit a specific immune response against the vaccine target in the subject.

The induced immune response can be used to treat, prevent, and/or protect against disease related to the vaccine target. For example, a SARS-CoV-2 vaccine to the subject can induce or elicit a specific immune response against the SARS-CoV-2 virus in the subject. The induced immune response provides the subject administered the vaccine with protection against the vaccine target, such as a SARS-CoV-2 vaccine provides resistance to SARS-CoV-2.

The induced immune response can include an induced humoral immune response and/or an induced cellular immune response. The induced humoral immune response can include IgG antibodies and/or neutralizing antibodies that are reactive to the antigen. The induced cellular immune response can include a CD8+ T cell response. The number of vaccine doses for effective treatment can be 1, 2, 3, 4, 5, 6, 7, 8, 9, or 10.

The vector can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration.

The vector can be administered prophylactically or therapeutically.

The vector can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997). The vector can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The vector can be delivered via a variety of routes. Typical delivery routes include parenteral administration, e.g., intradermal, intramuscular or subcutaneous delivery. Other routes include oral administration, intranasal, and intravaginal routes. The vector can be delivered to the interstitial spaces of tissues of an individual (Felgner et al., U.S. Pat. Nos. 5,580,859 and 5,703,055. The vector can also be administered to muscle, or can be administered via intradermal or subcutaneous injections, or transdermally, such as by iontophoresis. Epidermal administration of the vector can also be employed. Epidermal administration can involve mechanically or chemically irritating the outermost layer of epidermis.

The vector can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vector can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

The vector can be administered via electroporation, such as by a method described in U.S. Pat. No. 7,664,545. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359. The electroporation may be carried out via a minimally invasive device.

The vector may be used in imaging. For example, the vector may express a fluorescent protein.

The vectors may be used alone or in combination with other agents.

### EXAMPLES

### Example 1

### A Binary Self-Amplifying Nucleic Acid Platform Eliminates the Requirement for Lipid Nanoparticles in the Creation of Vaccines and Nanomedicines

Conventional mRNA-based vaccines played a crucial role in alleviating the strain on healthcare systems during the pandemic and reducing mortality rates. However, the initial mRNA expression approach had notable technological limitations. In this context, we introduce an advanced binary gene expression nanotechnology that exhibits exceptional performance. This nanotechnology can function as either self-amplifying mRNA or self-amplifying DNA. In comprehensive assessments, both innovative formats demonstrate stability after multiple freeze/thaw cycles and freeze-drying. They also sustain prolonged expression of model proteins, inducing strong immune responses involving humoral antibodies and cell-mediated T lymphocytes against the SARS-CoV-2 spike protein. A noteworthy departure from established mRNA nanomedicine practices is that neither of these formats requires encapsulation with lipid nanoparticles (LNP) to achieve enduring gene expression in vivo. This surpasses other delivery methods. Consequently, both formats outperform existing LNP-mRNA systems, simultaneously harnessing the potency of conventional mRNA and the efficient dosage of self-amplifying vectors. Moreover, they offer the simplicity, swift development, ease of storage, and convenient distribution associated with stable, freeze-dried plasmid DNA nanomaterial. Remarkably, vaccination with the naked self-amplifying DNA format results in durable and sustained antibody responses and holds demonstrated promise for curtailing viraemia, thus mitigating SARS-CoV-2 replication and transmission. This platform holds the potential to revolutionize nanomedicine applications, making them more effective, economical, and accessible. Its scope extends beyond vaccines to encompass novel avenues such as delivering immunotherapeutics and other biologics, marking a significant advancement in the field.

### Results

### Both Gemini-R and Gemini-D Induced Amplification in Transfected Cells

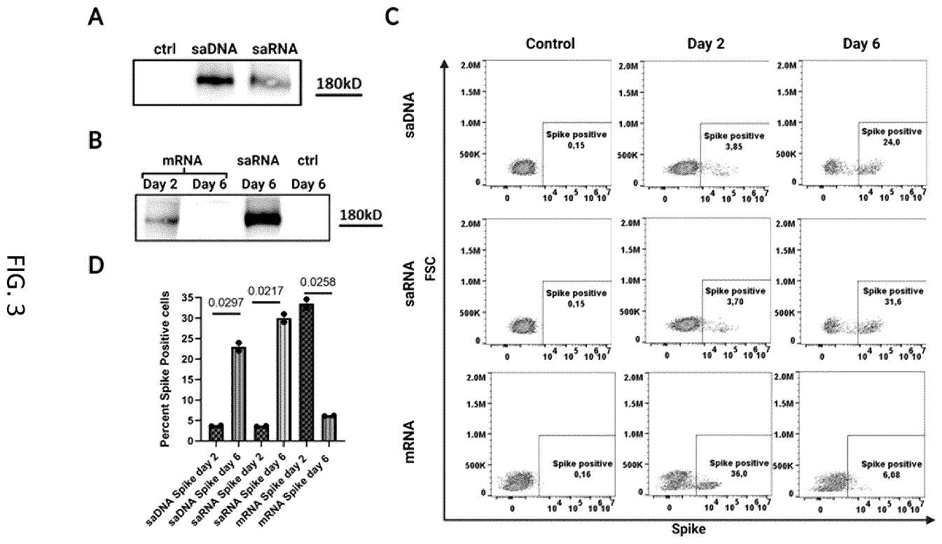

Gemini is a replicon-based self-amplifying dual expression vector containing a prokaryotic T7 promoter that can drive the transcription of mRNAs in vitro (Gemini-R) for uses in cells or tissues combined with a eukaryotic promoter that can faithfully transcribe mRNA after being delivered as plasmid DNA (Gemini-D) in cells or tissues (FIG. 1). As an initial validation of the fidelity of the Gemini design, we confirmed active amplification by using RT-PCR to measure the expression of negative-stranded mRNA in HEK293 cells transfected with either Gemini-R or Gemini-D (FIG. 2A, see also Materials and Methods).

PCR products of the expected size were detected as the nested PCR product of total RNA extracted from transfected cells with either Gemini-R or Gemini-D (FIG. 2B). Their sample source can be attributed to the presence of the PCR product from the first round of PCR. To verify the fidelity of the platform, it was determined that the production of the requisite negative (−) RNA strand from the transfected cells was not attributable to primer-independent effects, it was observed that when a gene-specific forward primer was omitted during cDNA synthesis, no PCR products were present in either the first PCR or in the subsequent nested PCR (FIG. 2B).

### Both Gemini-R and Gemini-D Drove Protein Expression in Transfected Cells

Validation that Gemini is capable of driving expression of a clinically relevant payload from either format was conducted by transfecting HEK293 cells with either Gemini-R or Gemini-D, both expressing spike protein from the B. 1.617.2 (Delta) variant of the SARS-CoV-2 virus.

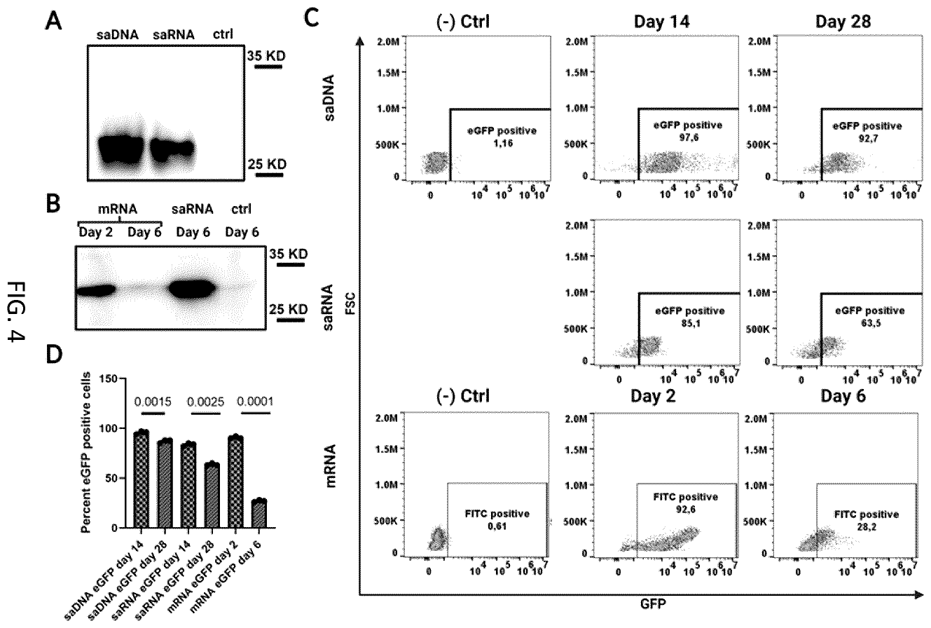

Western Blot analysis of protein expression at day 6 after transfection confirmed the presence of protein bands of the expected size (FIG. 3A). Western Blot results were subsequently reinforced by a flow-cytometric analysis of expression in transfected cells carried out over a time-course of 6 days. In the absence of any selection, by day 2 post-transfection, the Gemini-D driven form of Spike protein expressed in HEK293 cells revealed a weak positivity for spike protein expression (3.85% of the total cell population), but by day 6 the positive fraction had significantly increased (24.0% of the total population), suggesting self-amplification behaviour of Gemini-D (FIG. 3B-3D). Similarly, by day 2 post-transfection, the Gemini-R driven form of Spike protein expressed in HEK293 cells revealed a weak positivity for spike protein expression (3.70% of the total cell population), but by day 6 the positive fraction had significantly increased (31.6% of the total cell population), suggesting self-amplification behaviour of Gemini-R (FIG. 3B-3D). In contrast, cells transfected with a conventional LNP-encapsulated pseudouridine substitute mRNA encoding the B.1.617.2 (Delta) spike protein variant of the SARS-CoV-2 virus were 36.0% positive for the spike protein initially on day 2 post-transfection, but by day 6, there was a 6-fold reduction and the majority of the transfected cells had become negative (6.08%; FIG. 3B); demonstrating a rapid waning of expression in vitro.

eGFP Expression Induced by Gemini-D and Gemini-R in Transfected Cells and in Injected Mice

To compare in vitro payload expression capabilities of the saDNA and saRNA platform, HEK293 cells were transfected with either Gemini-R or Gemini-D expressing eGFP. Western blot analysis confirmed protein expression by yielding a protein band of the expected size at day 6 after transfection and sorting (FIG. 4A). Two days post-transfection, cells were cell-sorted to obtain 100% eGFP positive cells. A flow-cytometric analysis of such cells every week up to 6 weeks were performed (FIG. 4A, FIG. 4C). We found cells transfected with Gemini-D to be strongly positive on day 14 (98%). By day 28, this positive fraction had only marginally decreased to 93%, suggesting long-term expression from the Gemini-D self-amplificating format. In slight contrast, cells transfected with Gemini-R were 85% positive on day 14, and, by day 28, the corresponding fraction had significantly dropped to 64%. This suggests that expression from the Gemini-D is more stable than Gemini-R in transfected cells; the difference in the fraction of positive cells is statistically significant at both 14- and 28-days post-transfection (FIG. 4D) (p-value of unpaired t-test are 0.0007 and 0.0001, respectively). In contrast cells transfected and sorted for eGFP expressing a conventional LNP-encapsulated pseudouridine substitute mRNA encoding eGFP are initially most positive for eGFP (92.6%) but by day 6 (FIG. 4C) there is a rapid reduction in protein expression as detected by Western blotting (FIG. 4B), and nearly a 3-fold reduction in detectable eGFP expressing cells (28.2%) detected by flow cytometry (FIG. 4C). The p-value of unpaired t-test between day 2 and 6 is 0.0001 (FIG. 4D).

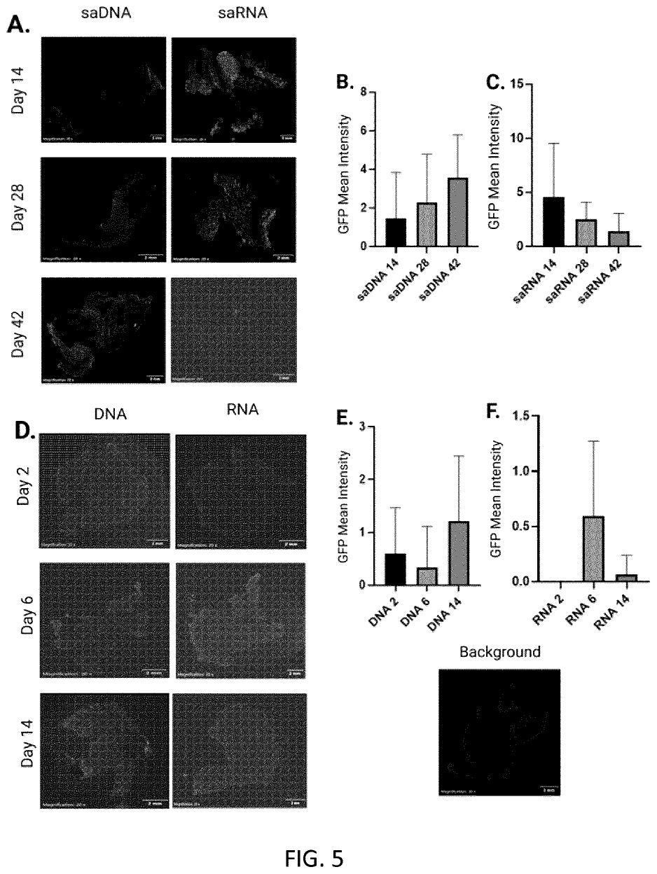

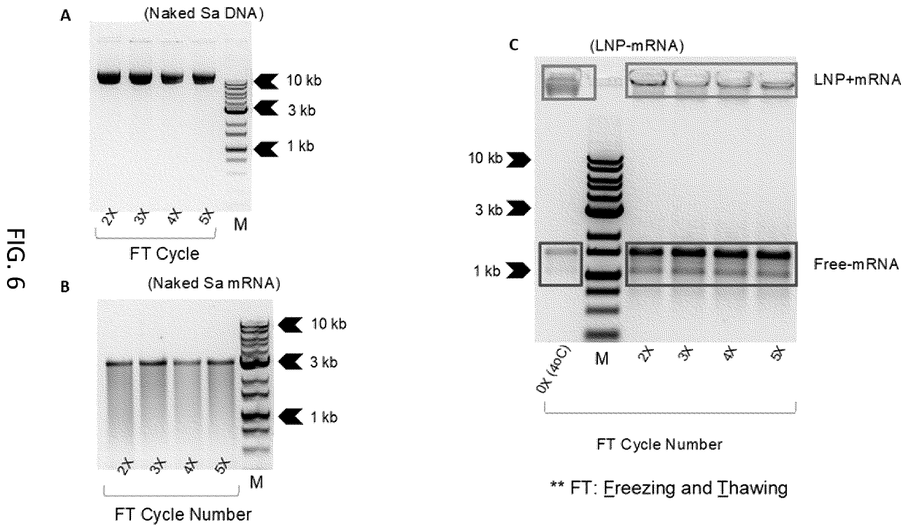

To evaluate such expression dynamics in vivo, mice were intramuscularly injected with the same Gemini-R or Gemini-D encoding eGFP. Injected leg muscles were collected at three different time points (14-, 28- or 42-days post-injection, n=3 per day of sacrifice), frozen and subsequently sectioned (as described in the Materials and Methods), then assessed for native eGFP expression both qualitatively (FIG. 5A) and quantitatively (FIG. 5B, FIG. 5C). Compared to the negative control, all time points except the day 14 for Gemini-D and day 42 for Gemini-R are less than p<0.05. For Gemini-R, high levels of eGFP were observed 14 days post injection, with levels appearing reduced at subsequent time points. On the other hand, expression levels from Gemini-D continued increasing and appeared highest at the latest time point, 42 days after injection. Finally, both the conventional plasmid DNA (FIG. 5D, 5E) and LNP-mRNA (FIG. 5D, 5F) drive lower eGFP expression for lesser durations than either Gemini-R or Gemini-D after injection into the muscle of mice.

Overall, the expression of eGFP from Gemini-R and Gemini-D has been demonstrated to persist for over 28 days, with Gemini-D being still highly active and potentially increasing in expression in vivo after 42 days while gene expression using conventional mRNAs diminish after a few days. The results with Gemini are consistent with the duration of gene expression achieved by viral vector vaccine platforms such as the ones based on recombinant adenoviruses or vaccinia virus (13, 14). Thus, though Gemini-D is a non-viral based plasmid format, its self-amplifying capacity allows it to last some 40 days in vivo. The data also suggest that Gemini-R may be useful for delivery and expression of therapeutic payloads for 4-weeks while Gemini-D may be useful for applications that require longer periods of expression.

### Neither Gemini-R Nor Gemini-D Induced Significant Genomic Integration

We utilized gel-based methods to separate genomic DNA from extrachromosomal DNA (15) and the properties of restriction enzyme AscI to detect genomic integration in muscle (16).

While it cleaves DNA derived from prokaryotic cells, AscI selectively ignores restriction sites that are CpG methylated in eukaryotic cells:

5′ G G↓C G C G C C 3′
3′ C C G C G C↑G G 5

We used it and established agarose gel procedure to distinguish between integrated and extrachromosomal nucleic acids to estimate the in vivo frequency of Gemini-R and Gemini-D integration in the genome of mouse muscle cells (see Materials and Methods).

While the Gemini-R format showed no detectable integration in the muscle injection site, the number of Gemini-D integrated copies was determined to be less than 3×10⁻⁶ per cell genome (see FIG. 5D). Such integration rate in muscle tissue is significantly lower than that of plasmid DNA, 5×10⁻⁵ (17), or adenoviruses, 6.7×10⁻⁵ (18). As recombinant adenoviruses are widely used clinically and considered one of the safest vaccine vectors available (18), this result demonstrates an excellent safety profile for Gemini-D and allays fears of induced genetic abnormalities, such as those resulting from lentiviruses and other commonly used retroviral expression systems More detailed comparisons are shown in Table below.

| | Spontaneous | |
|---|---|---|
| Organism/Vector | Mutation or Integration Rate | References |
| Human | 3.84 germline mutation per genome | (31, 32) |
| | per generation | |
| | 89.6 somatic mutation per genome | |
| | per generation | |
| Mouse (C57/B6) | 0.945-1.46 germline mutation per | (31, 32) |
| | genome per generation | |
| | 1188 somatic mutation per genome | |
| Adenovirus | 6.7 × 10⁻⁵ integrations per genome | (30) |
| | in transduced hepatocyte | |
| Plasmid DNA vaccines (IM) | 5 × 10⁻⁵ integrations per genome | (29) |
| Self-amplifying DNA platform | 3 × 10⁻⁶ integrations per genome | |
| in this paper (Gemini-D) | | |
| Self-amplifying RNA platform | 0 integrations per genome | 0 |
| in this paper (Gemini-R) | | |

Thus, overall, Gemini-R was demonstrated not to integrate into the host genome while Gemini-D was determined to be less than 3×10⁻⁶ per cell genome, lower than other clinically approved vector platforms and many orders of magnitude lower than the spontaneous somatic mutation frequency (see above Table) in mice or humans (19, 20) establishing this as one of the safest platforms yet created.

Stability of Gemini-D and Gemini-R Compared to a Conventional LNP-Encapsulated mRNA Vaccine

The stability of vaccine platforms determines both their shelf-life, distribution, and transportation chain, and ultimately their efficacy. In order to test this we subjected the Gemini-D and Gemini-R and a conventional LNP-pseudouridine substituted mRNA analogous to the Moderna vaccine all encoding spike protein from the B.1.617.2 (Delta) variant of the SARS-CoV-2 virus. The stability of Gemini-D (FIG. 6A), Gemini-R (FIG. 6B) and LNP-mRNA (FIG. 6C) was compared after many cycles of freezing and thawing at −80° C. and analysed at room temperature (RT) by agarose gels electrophoresis. It should be noted the LNP-mRNA was shipped with blue ice during the 3-day of transit. Upon the arrival, it was kept at 4° C. overnight and utilized for the stability test the next day. The stability of the formulated LNP-mRNA and free nucleic acids and lipid structures were also examined by agarose gels electrophoresis. For the experiment, each vaccine sample (2 μg for Naked Gemini-D/Gemini-R and 1 μg for LNP-mRNA respectively) was subject to the freezing and thawing up to 5 times in total (sample number one is frozen and thawed twice and subsequent sample number indicates the samples frozen and thawed 3×, 4×, and 5× respectively). The Gemini-D, Gemini-R samples were mixed with 6×DNA or 2×RNA loading dyes and loaded into well of 1.2% agarose gel while LNP-mRNA samples were loaded to the 0.8% agarose gel to assess the core structures of large MW LNP. The gels were run at 80V for about 40 min with TAE buffer. The gel images were documented using the gel doc system at our research facility. We observed that Naked Gemini-D and Gemini-R maintained their integrity even after 5-cyles if freezing-thawing. However, while LNP-mRNA also maintained its mRNA integrity, but it was released from the core lipid structures of LNP after a single cycle of free-thaw resulting in free LNP exclusive of liberated mRNA which likely results in the loss of all biological functions or benefits that LNP-encapsulation may offer.

Each vaccine was subjected to repetitive round of freezing (−80 c) and thawing (RT) and then analysed by agarose gel electrophoresis. While the nucleic acid components of all three vaccines were undegraded and remained stable after 5-cycles of free-thaw, the conventional LNP-pseudouridine substituted mRNA vaccine appears to be composed of a significant portion of free mRNA upon arrival from the manufacturer, formulated LNP-mRNA appears unstable and is disrupted after a single cycle of freezing and thawing thereby liberating its mRNA cargo.

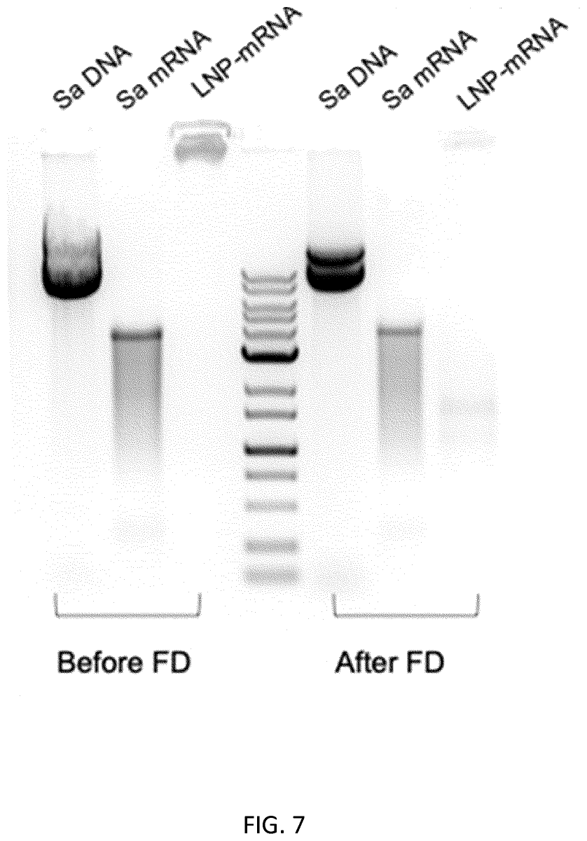

To assess the effect of Gemini-D, Gemini-R and LNP-mRNA after Freezing-Drying (FD; lyophilization), approximately 2.5 μg of three different vaccines were kept in a total volume of 100 μl of Tris-cl buffer, water and PBS, respectively (FIG. 7). They were freeze-dried in a Labconco's lyophilizer using the recommended parameters by the manufacturer. The parameters were as follows: Solidification/Eutectic temperature was −30 to −40° C., pre-freeze temperatures were −40 to −50° C. and vacuum set point was 0.12 to 0.04. The entire process was completed in 4 hours. Subsequently, dried material was kept at −20° C. overnight. The next day, they were reconstituted in Tris-cl, water and PBS again on ice and mixed with DNA or RNA loading dye (saRNA and LNP-mRNA). They were run on 0.8% TAE-agarose gel at 85V for 40 mins along with non-lyophilized counterparts and documented on a gel doc system. The data demonstrates that both the Gemini-D, and Gemini-R formats can be reconstituted after freeze-drying but the LNP-mRNA disassembles into free LNP and free mRNA. Reinforcing the superiority of the Gemini-D, and Gemini-R formats over LNP-mRNA formulations.

Significant Antibody Response were Induced by Both SARS-CoV-2 Spike Gemini-R and Gemini-D Vaccine Formats

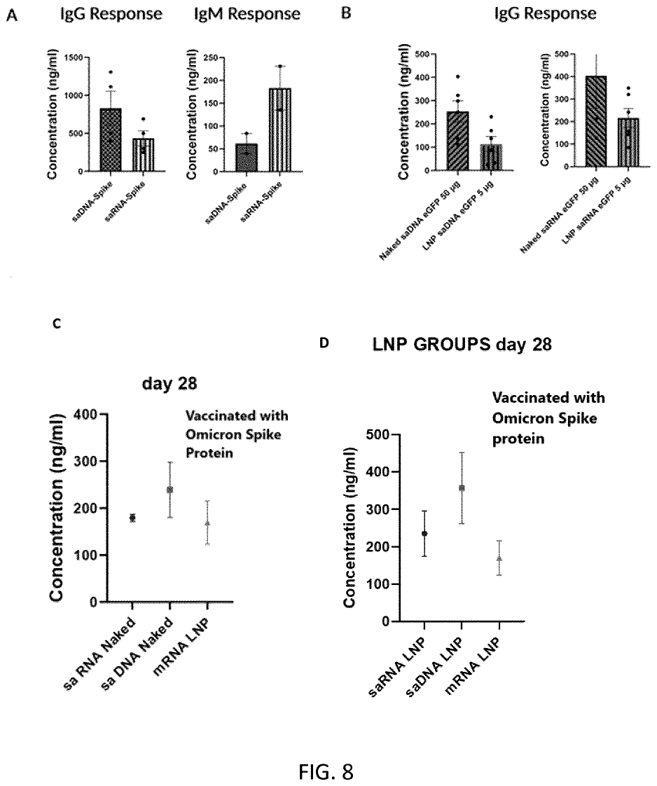

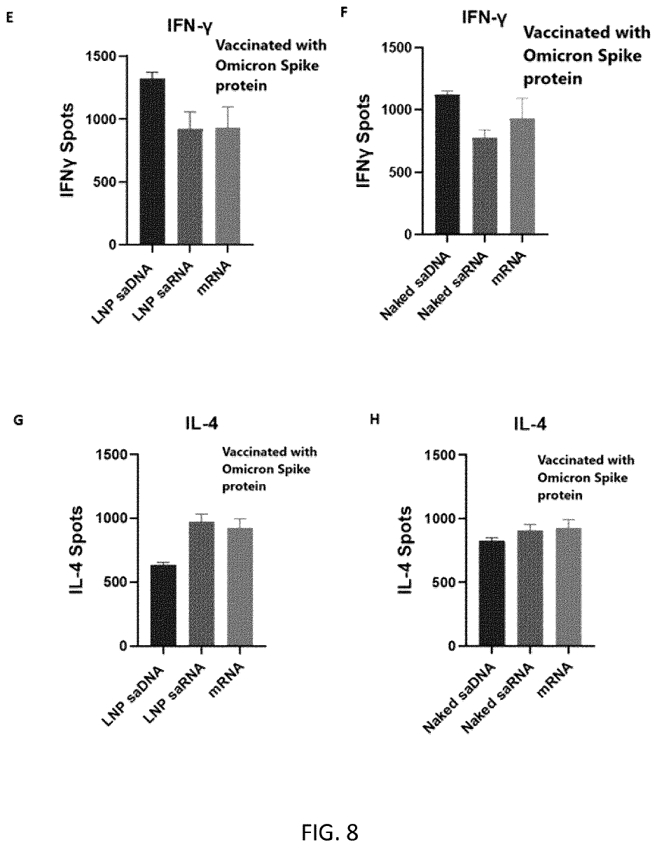

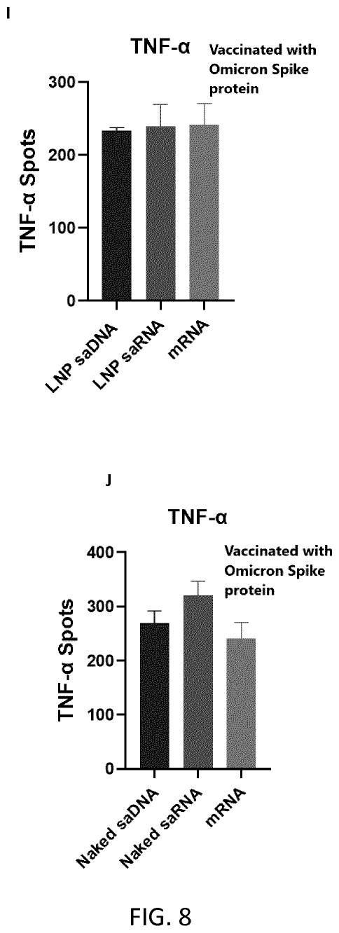

The antibody response derived against a relevant vaccine payload was evaluated in mice. Gemini-R and Gemini-D vectors expressing the B.1.617.2 (Delta) spike variant of the SARS-CoV-2 virus were LNP-encapsulated (see Materials and Methods) and 5 μg (the optimal dose) of either vaccine formulation was injected into K18-hACE2 transgenic mice expressing the human ACE-2 SARS-CoV-2 receptor according to the vaccination protocol outlined in materials and methods. All the treatment groups were statistically different than the unvaccinated control group (ANOVA, p=0.0082). Consistent with the flow cytometry findings described above, the IgG response to the Gemini-D format of the vaccine in serum extracted from inoculated mice on day 28 (FIG. 8A) was found to be significantly greater (p=0.0046) when compared to the unvaccinated control group, while the IgG response to the Gemini-R format of the vaccine was found to be lower than that achieved with the Gemini-D format (p=0.1019) compared to the unvaccinated control group. In contrast, the IgM response (FIG. 8A) demonstrated opposite behavior, with significantly higher IgM levels generated by the Gemini-R format of the vaccine (p=0.0420; here the R-Gemini group was compared to the unvaccinated group with unvaccinated background being subtracted from both the groups), and a lower IgM response generated by the Gemini-D format of the vaccine (p=0.1525, here the D-Gemini group was compared to the unvaccinated group with unvaccinated background being subtracted from both the groups). Finally, neither vaccine format induced a significant IgA antibody response nor any observable toxicity resulting in weight loss, a parameter of safety and toxicity in mice (p>0.05, data not shown).

Comparable absolute quantitative estimates in the clinical literature are limited, as most estimates are based on international units. However, the few available sources of absolute quantitation of antibody responses such as (21), where they tested several thousand patients, support the findings that either LNP-encapsulated Gemini-R and Gemini-D format elicit robust antibody responses; in quantitative terms (ng/ml). Overall, a single dose of 5 μg of either the LNP-encapsulated Gemini-R and Gemini-D formats (FIG. 8A) elicit significantly greater IgG concentrations (400-800 ng/ml) compared to those observed in patients mounting immune responses to SARS CoV-2, which on average reach ˜200 ng/ml. Finally, following intramuscular injections, Gemini-R may have advantages in vaccines where IgM has the greatest clinical value while Gemini-D may find its greatest use in applications where immunity to specific pathogens require IgG responses. It is conceivable that these differences may be due to differential Toll-like receptor (TLR) signally pathway for RNA and DNA (22). Thus, utilizing different Gemini formats may provide the opportunity to “tune” immune responses towards an IgG or IgM dominant immune response.

### LNP Encapsulation are not Necessary for the Immunogenicity of Either Gemini-D- or Gemini-R Vaccine Formats

Current paradigms for RNA delivery require encapsulation of either mRNA or saRNA through expensive and technically demanding formulations. In contrast to this, protecting saRNA from RNAse digestion, either on the interior formulated LNP or on the exterior of pre-made cationic LNP particles (23) protects saRNA from RNAse digestion and, after vaccination, induces a statistically equivalent amount of antibodies against the HIV-1 Env gp140 protein used as a model antigen (23).

In the context of this study, we thought it prudent to addressed whether LNP encapsulation is necessary for successful delivery and performance of the vaccines based of either Gemini-D or Gemini-R using eGFP as a “model antigen”. All the treatment groups were statistically different than the unvaccinated control group (ANOVA, p=0.0134). Gemini vaccines expressing eGFP were tested (FIG. 8B) and showed that higher doses (50 μg) of Naked Gemini-D performs better (p=0.0129) when compared with 5 μg of LNP-encapsulated Gemini-D (p=0.1983). We achieved similar results were consistent for 50 μg of Naked (p=0.0420) and 5 μg of encapsulated Gemini-R (p=0.3269) expressing eGFP as a model antigen when compared with control groups.

Comparison of antibody responses among different vaccine formulations was conducted, specifically evaluating the IgG response in mouse serum 28 days after injection. The vaccines under consideration were Naked D-Gemini (100 μg), R-Gemini (25 μg), and LNP-encapsulated mRNA (5 ug). A normal saline injected control was also included for reference. Background control in enzyme-linked immunosorbent assays (ELISA) utilized normal serum from age-matched unvaccinated K18hAce2 transgenic mice. A one-way analysis of variance (ANOVA) encompassing all groups, including the normal saline control, was performed. Subsequent to ANOVA, Dunnett's post-hoc test facilitated multiple comparisons between the normal saline group and each vaccine group. The resulting p-values were as follows: 0.0712 (Naked saRNA), 0.0082 (Naked saDNA), 0.0970 (LNP mRNA) (FIG. 8C). Likewise, a comparison of antibody responses was undertaken for LNP-encapsulated D-Gemini (5 μg), R-Gemini (5 μg), and LNP-encapsulated mRNA (5 μg) vaccines in mouse serum 28 days post-injection, assessing the IgG response. This evaluation also incorporated the normal saline injected control and utilized background control from unvaccinated K18hAce2 transgenic mice. The procedure encompassed a one-way ANOVA analysis for all groups, including the normal saline control, followed by multiple comparisons using Dunnett's post-hoc test. The corresponding p-values were: 0.1675 (LNP saRNA), 0.0079 (LNP saDNA), 0.3757 (LNP mRNA) (FIG. 8D). Subsequently, the IFN-γ ELISPOT response was examined in mouse spleen cells post-harvest for LNP-encapsulated D-Gemini, LNP-encapsulated R-Gemini, and mRNA vaccines. Similar to the previous analyses, a one-way ANOVA was conducted for all groups, including normal saline control, followed by multiple comparisons utilizing Dunnett's post-hoc test. The calculated p-values were as follows: 0.0006 (LNP saDNA), 0.0370 (LNP saRNA), 0.0461 (LNP mRNA) (FIG. 8E). A comparable evaluation was undertaken for IFN-γ ELISPOT responses in Naked D-Gemini, Naked R-Gemini, and mRNA vaccines within mouse spleen cells post-harvest. The analytical approach mirrored previous methods, involving a one-way ANOVA analysis for all groups, including normal saline control, and subsequent multiple comparisons using Dunnett's post-hoc test. The obtained p-values were: 0.0050 (Naked saDNA), 0.3271 (Naked saRNA), 0.0461 (LNP mRNA) (FIG. 8F). Similarly, the comparison extended to IL-4 ELISPOT responses for LNP-encapsulated D-Gemini, LNP-encapsulated R-Gemini, and mRNA vaccines within mouse spleen cells post-harvest. The analysis followed the established pattern, with a one-way ANOVA encompassing all groups, including normal saline control, and multiple comparisons via Dunnett's post-hoc test. The p-values obtained were: 0.9730 (LNP saDNA), 0.0004 (LNP saRNA), 0.0088 (LNP mRNA) (FIG. 8G). Further examination included the comparison of IL-4 ELISPOT responses for Naked D-Gemini, Naked R-Gemini, and mRNA vaccines within mouse spleen cells post-harvest. The process remained consistent, with a one-way ANOVA analysis for all groups, including normal saline control, and multiple comparisons using Dunnett's post-hoc test. The calculated p-values were: 0.2048 (Naked saDNA), 0.0054 (Naked saRNA), 0.0088 (LNP mRNA) (FIG. 8H). Lastly, the comparison encompassed TNF-α ELISPOT responses for LNP-encapsulated D-Gemini, LNP-encapsulated R-Gemini, and mRNA vaccines within mouse spleen cells post-harvest. The established methodology included a one-way ANOVA analysis for all groups, including normal saline control, and subsequent multiple comparisons using Dunnett's post-hoc test. The derived p-values were: 0.9906 (LNP saDNA), 0.0476 (LNP saRNA), 0.9813 (LNP mRNA) (FIG. 8I). Parallelly, the analysis extended to TNF-α ELISPOT responses for Naked D-Gemini, Naked R-Gemini, and mRNA vaccines within mouse spleen cells post-harvest. The analytical framework followed the familiar pattern of a one-way ANOVA analysis for all groups, including normal saline control, and multiple comparisons using Dunnett's post-hoc test. The resultant p-values were: 0.9128 (Naked saDNA), 0.0011 (Naked saRNA), 0.9813 (LNP mRNA) (FIG. 8J).

There was no statistical difference between Gemini-D and Gemini-R formats for Naked and LNP-encapsulated vaccine and clearly shows that dosing alone can achieve identical T cell responses for Naked forms of Gemini that are achieved with LNP-forms of Gemini.

Vaccination with Naked saDNA or saRNA Formats Resulted in Durable Antibody Responses

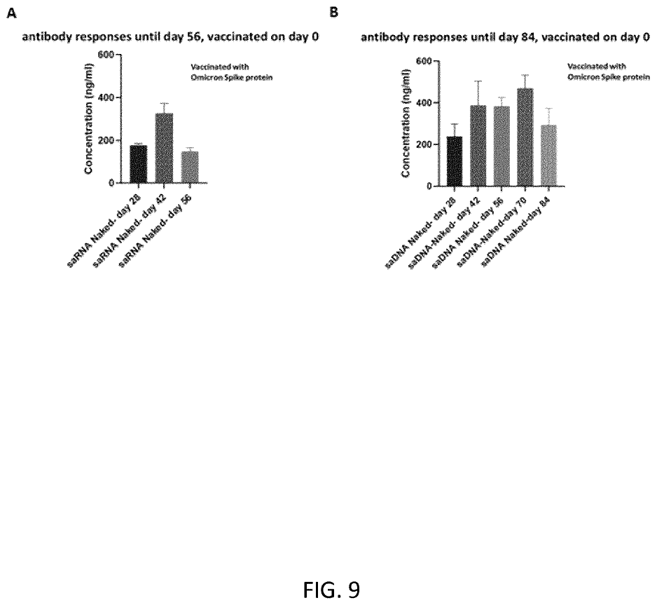

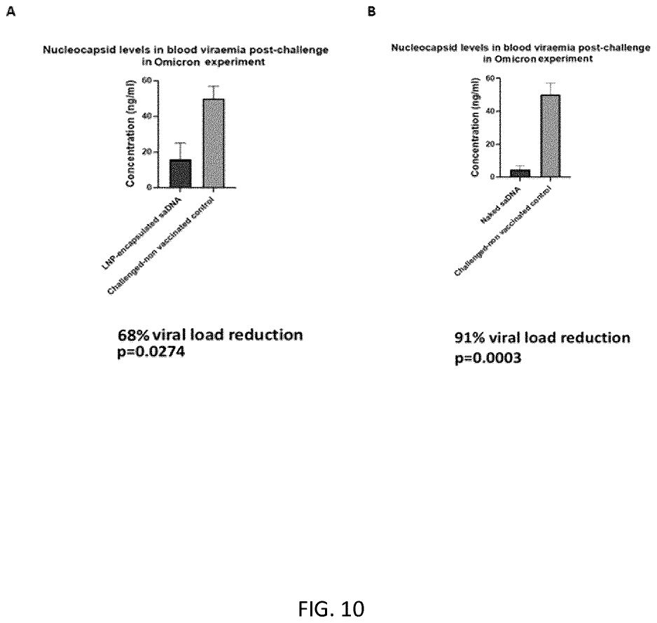

To assess if vaccination with either Naked saDNA or saRNA formats gave longer more durable antibody responses a time-course was undertaken to assess this. The animals were vaccinated with Naked saRNA or saDNA vaccines expressing Omicron-Spike and the serum antibody responses were measured over time. Responses in Naked saRNA vaccines were demonstrated to be durable up to 42 days (FIG. 9A) before they began to wane. However, responses to payloads delivered by Naked saDNA vaccines were demonstrated to be durable until at least 70 days before they began to wane (FIG. 9B). Thus, the antibody responses elicited by Naked saDNA vaccines potentiate a longer more durable response than the Naked saRNA vaccines and were therefore selected for viral challenge experiments.

Immunization with SARS-CoV-2 Spike-Specific saDNA in Mice Elicits Reduces Viraemia Post-Challenge.

Finally, to conclusively establish if the Naked saDNA could outperform the LNP-version of the saDNA and therefore establish if the Naked DNA format could replace the need for inclusion of LNP, viral challenge experiments were undertaken to compare LNP encapsulated or Naked Omicron-Spike saDNA, After the susceptibility of K18-hACE2 mice to SARS-CoV-2 was established, they were administered nucleic acid vaccines intramuscularly on days 0 and 28. Post a 5-day interval from the second dose, the mice were exposed to SARS-CoV-2 challenge and then euthanized five days following the challenge. The viraemia levels in their blood were analyzed by examining serum samples from the challenged mice. The mice received vaccinations involving LNP encapsulated or Naked Omicron-Spike saDNA and subsequently, they were exposed to Delta virus challenge respectively. The concentration of nucleocapsid protein was measured in the blood viraemia samples collected from mice vaccinated with saDNA-LNP encapsulated or Naked Omicron Spike vaccines, five days post-challenge. The amount of nucleocapsid in blood viraemia samples collected from mice vaccinated with saDNA-LNP encapsulated vaccines was measured five days after the challenge. A statistically significant difference was observed between the vaccinated group and the control group that didn't receive vaccinations, as determined by an unpaired T-test (p=0.0274) (FIG. 10A). The amount of nucleocapsid in blood viraemia samples collected from mice vaccinated with saDNA-Naked vaccines was measured five days after the challenge. A notable distinction was found between the vaccinated group and the control group that was not vaccinated, as confirmed by an unpaired T-test (p=0.0003) (FIG. 10B).

### Discussion

Our study demonstrates that the Gemini platform is useful for the creation of recombinant vaccines and potentially other payloads that may be of use therapeutically and provides several benefits when compared with other platforms such as the conventional recombinant mRNA and DNA technologies.

There are clear advantages of the dual format of Gemini, as it safely combines the flexibility and power of RNA platforms with the much greater stability and ease of manufacturing of DNA constructs. In particular, the self-amplifying DNA plasmid format used in the Gemini-D format can be very rapidly created, scaled-up in very large conventional manufacturing batches, resulting in better standardization, without being impeded by the production bottlenecks incurred by the highly technical manufacturing procedures required for conventional RNA vaccines, nor the inclusion of additional, highly specialized LNP technologies; these favorable properties would assure a swifter response than currently imagined to a future pandemic. In addition, Gemini-D is similarly convenient in terms of distribution and thermal stability storage as it is stable, can be lyophilized and thus it does not need ultra-low storage temperatures during transportation. Furthermore, the dual expression platform offers the ability to choose and directly compare either a saRNA or saDNA platform while retaining the same payloads. Thus, Gemini may overcome issues associated with vaccine stability, attributed to the requirements of prolonged ultra-low temperature storage, avoiding logistical and practical concerns associated with the world-wide distribution of vaccines (24).

In this study, the B.1.617.2 (Delta) spike protein variant of the SARS-CoV-2 virus and the B. 1.1.529 (Omicron) Spike variant of SARS CoV-2 Spike and the eGFP gene were chosen as “model” antigenic payloads to establish the proof-of-concept of both the Gemini-D and Gemini-R due to the current interest in SARS-CoV-2 vaccines and the established utility of eGFP as faithful reporter protein antigen. Both Gemini-R and Gemini-D can express payload proteins for over 28 days in vivo in mouse muscle, which is similar to the duration achieved by viral expression vectors such as those based on adenoviruses or vaccinia virus. On the other hand, both the expression level and duration of expression of conventional plasmid DNA and LNP-mRNA driving eGFP expression is significantly lower than either Gemini-R or Gemini-D after injection into the muscle of mice.

Furthermore, it should be noted that transfection of HEK293 cells with a conventional LNP-pseudouridine substituted mRNA analogous to the Moderna vaccine intitiates expression by 2 days but the expression of spike protein from the B. 1.617.2 (Delta) variant of the SARS-CoV-2 virus or eGFP diminishes 3-6 fold by only 6-7 days of transfected cells. Early studies on the initiation of humour immunity by B-lymphocytes established that lower antibody responses were noted in animals receiving exposure to antigen for less than 4 days (21). Subsequent studies on T-lymphocyte responses to viruses demonstrated that a similar minimum duration of temporal exposure to the antigen of 4 days was necessary to generate maximal cell-mediated immunity and immunological T-lymphocyte memory (22). Our in vitro work demonstrates that self-limiting conventional LNP-mRNA used in widely distributed SARS-CoV-2 vaccines could result in rapidly waning antigen expression in vivo with a timescale of a few days. Therefore, it is an open question whether the transient antigen expression provided by such delivery systems might be related to the rapid decline in immunity following vaccination against SARS-CoV-2 with LNP-mRNA (23).

In addition, a key and elegant innovation in the recent mRNA vaccine revolution was the inclusion of pseudouridines during transcription to create a highly expressed, non-immunogenic, non-inflammatory platform (25). Paradoxically, the LNPs used to deliver the mRNA vaccines have been found to be highly pro-inflammatory and thus, may contribute to some of the observed side effects of these LNP-encapsulated vaccines (26). Both Gemini platforms are efficacious without the need for LNP-encapsulated and therefore reduce the potential for adverse reactions due to the noted highly inflammatory nature of LNPs (26). The literature allows the accurate comparison the performance of the Gemini platform to be compared to conventional mRNA expression systems. Comparable studies have used endpoint titer calculations in mRNA-1273 vaccination studies in K18-hAce2 transgenic mice (27, 28). Endpoint titer is defined by the highest dilution of serum which is 2 standard deviations above the background in the ELISA-based antibody assays. Most of the articles used serial ten-fold dilutions of serum in their ELISA assays and reported endpoint titres of 1:10000 or 1:100000-fold (27-29). Compared to the unvaccinated control which is positive at 1:100 dilution (2 standard deviations above the background), the effective endpoint recorded to be from 1:64 to 1024 (30) serum dilution or titers ranging from 100-to-1000-fold over the background (31, 32). This establishes that the highest positive titre at 10²-10³-fold compared to the unvaccinated control. In our studies we use three-fold dilutions of serum instead of 10-fold dilutions. Our highest dilution of serum tested is 1:2160 and our endpoint titer is between 1:720 and 1:2160 dilution of serum placing it in the 10³-fold range. The performance of both Gemini platforms without LNPs, are therefore comparable or exceed titres described for mRNA-1273 vaccinations. Furthermore, it is of interest to note that studies have shown that a majority of uninfected adults show preexisting antibody reactivity against SARS-CoV2, potentially because of prior exposure to other human coronaviruses including SARS-CoV-1, HKU1, N63L, or 229E (but not OC43) resulting in antibody cross-reactivity (33). Thus, assessment of antibody titres between pre-clinical mouse models where exposure to SARS-CoV-2 or vaccination with components of SARS-CoV-2 constitutes a primary immune response compared to clinical data where exposure to SARS-CoV-2 or vaccination with components of SARS-CoV-2 constitutes a secondary immune responses is fraught with difficulties. Nevertheless, this renders the titres we observe in mice after a single immunization even more impressive.

We also quantitated the T lymphocyte responses achieved after vaccination with either LNP or Naked forms of Gemini-R (saRNA) and Gemini-D (saDNA) encoding the B. 1.617.2 (Delta) spike variant of the SARS-CoV-2 that were injected intramuscularly at 5 μg for the LNP forms and 200 μg for the Naked forms per mouse. We observed significant increases in gamma-interferon expressing T cells assessed by ELISPOT analysis that recognized peptide epitopes contained in B.1.617.2 (Delta) spike protein. Furthermore, we observed no differences between the T cell responses elicited by either form of the LNP or Naked vaccines and therefore dosing itself can overcome the need for inclusion of LNPs. In general, the saDNA constructs trended to be superior in generating Th1 responses and the saRNA constructs trended better in generating Th2 responses. In most assays both tended to perform better than the conventional LNP-mRNA format (FIG. 8 B-H).

To evaluate whether vaccination with either Naked saDNA or saRNA formats could lead to longer-lasting and more robust antibody responses, a time-course study was conducted to assess this aspect. Animals received vaccinations using either Naked saRNA or saDNA vaccines that expressed the Omicron-Spike protein, and the levels of antibodies in their serum were monitored over time. The results showed that antibody responses in animals vaccinated with Naked saRNA remained strong for up to 42 days (as shown in FIG. 9A) but started to decline afterward. In contrast, animals vaccinated with Naked saDNA exhibited robust antibody responses that remained durable for a longer period, extending up to 70 days (as indicated in FIG. 9B). Therefore, it can be concluded that Naked saDNA vaccines induce a more prolonged and sustained antibody response compared to Naked saRNA vaccines. In the context of many other applications in nanomedicine, a shorter duration of biologic expression may be advantageous however, in the context of vaccines it appears that provoking longer and more durable antibody responses have significant advantages.

Finally, in order to assess if the novel saDNA format, that may have the highest potential for vaccines based on its physical characteristics and durability of antibody responses, could reduce blood viraemia of SARS CoV-2 we undertook viral challenge experiments to compare the Naked and LNP-encapsulated SARS CoV-2 Spike saDNA vaccines. We find that both formulations reduced viraemia significantly with the Naked saDNA (91% reduction, FIG. 10A) outperforming the LNP-encapsulated SARS CoV-2 Spike vaccine (68% reduction, FIG. 10B). This finding offers encouragement for more thorough testing of the Naked saDNA platform's ability the reduce the replication and spread of SARS CoV-2 in the absence of LNPs and in so doing, illicit sterilizing immunity.

The findings in this study appear to contradict the accepted wisdom on previously described nucleic acid platforms (23) by offering the encouraging possibility that LNPs may be omitted altogether in future self-amplifying vaccines and simple dosing might be used to dispense with the technically demanding LNP encapsulation of vaccine payloads delivered through either Gemini-R and Gemini-D. Furthermore, the implications are particularly important for Gemini-D saDNA-based vaccine format, which can be rapidly prepared, in unlimited amounts, utilizing a simple plasmid preparation without the need for LNPs of any kind for its functionality as a vaccine.

Much-underappreciated aspects of vaccine manufacturing are critical limitations in scalability, stability, storage and distribution. In this light, we assessed the thermal stability of Gemini-D and Gemini-R and a conventional LNP-pseudouridine substituted mRNA vaccine encoding spike protein from the B.1.617.2 (Delta) variant of the SARS-CoV-2 virus. We find that both Naked forms the Gemini-D and Gemini-R are extremely stable after 5-cycles of freezing and thawing and while the mRNA component of the LNP-mRNA also appears to be stable we observed that the formulated LNP-mRNA disintegrates after a single freezing and thawing cycle liberating free LNP and free mRNA. Surprisingly, this simple analysis calls into question the labiality of the LNP-mRNA vaccines that are in global circulation because all are transported at −20-80 C, and all are subject to thawing prior to use as vaccines in people. If our observations are reinforced in the analysis of clinical batches of vaccine LNP-mRNA or indeed other LNP-payload formulations, caution should be taken interpreting the added benefit of LNP-encapsulation when the workflow includes a freeze-thaw cycle. The additional superior characteristics of both Gemini platform is the ability to freeze dried in either RNA or DNA formats and be reconstitute in an aqueous solution. This is significant advance on all other platforms including LNP-mRNA platforms and viral platforms that do not survive freeze drying. The importance of this to the field of vaccinology and indeed to biotechnology in general, should not be understated as improving stability, storage and thereby “self-life” has been to major goal of vaccine and pharmaceutical developers for decades and will allow the stockpiling of vaccines and therapeutics well in advance of disease outbreaks or therapeutic applications and allow the long-term storage and rapid distribution of vaccines and therapeutics for a myriad of diseases.

Taken together, the Gemini platform possesses attractive properties with respect to storage and safety profiles that likely exceed other recombinant vaccine platforms, while eliminating the need for LNP encapsulation. Finally, it is impactful that non-encapsulated or Naked form of Gemini-D, the self-amplifying DNA plasmid format, is simple enough to be created, throughout the globe, in laboratories with limited technical resources. In the longer term, the Gemini-D platform may lead to a true democratization in the creation, manufacturing, and distribution of vaccines and nanomedicines.

### Materials and Methods

### Vector Synthesis

FIG. 1A illustrates the map of the vector system discussed in this paper, hereinafter referred to as Gemini 1.0. It is based on the T7-VEE-GFP plasmid a very gift from Professor Steven Dowdy, at Department of Cellular & Molecular Medicine, University of California, San Diego School of Medicine, 9500 Gilman Drive, La Jolla, CA 92093-0686, USA (34). It consists of the NSP1-4 genes from Venezuelan equine encephalitis (VEE) virus, an origin of replication site, a bacterial promoter (26S subgenomic promoter), an Ampicillin resistance (AmpR) gene acting as a selection marker for bacterial culture, a T7 promoter to recruit T7 RNA polymerase for saRNA synthesis, and a human CMV enhancer/promoter, for use as a DNA or RNA vector in humans. The CMV promoter was cloned into the T7-VEE-GFP plasmid by Synbio Technologies. For subsequently the B. 1.1.529 (Omicron) Spike variant of SARS CoV-2 and B.1.617.2 (Delta) spike variant of the SARS-CoV-2 virus and eGFP sequences.

saDNA and saRNA Preparation, Conventional Plasmid DNA and mRNAs

The Gemini 1.0 vector was transformed into DH5a Competent E. Coli (NEB, C2987) and plated onto Luria-Bertani (LB) agar containing Ampicilin for selection; this was followed by overnight culturing in LB broth at 37° C. Plasmid DNA was extracted according to the EZ10 Plasmid DNA Minipreps Kit protocol (BioBasic, BS6149). To prepare the saRNA, the Gemini 1.0 plasmid underwent in vitro transcription using T7 RNA polymerase (NEB, M0251L), followed by in vitro 5′ capping and 3′ polyadenylation. FIG. 1 describes the self-amplifying platform pathways and in vitro replication process for both the DNA and RNA forms.

mRNA Preparation

Pseudouridine substitute LNP-encapsulated mRNAs encoding eGFP (Cat #PM-LNP-21) or LNP-B. 1.617.2 (Delta) spike protein variant of the SARS-CoV-2 virus (Cat #PM-LNP-12) mRNA purchased from Promab Biotechnologies, 2600 Hilltop Dr Building B, Suite C320, Richmond, CA 94806, United States. These LNP's were formulated by Prolab with, SM-102, DSPC, cholesterol, and DMG-PEG2000 at optimal molar concentration for a high rate of encapsulation and efficient mRNA delivery.

### Lipid Nanoparticles (LNPs) Formulation of Gemini-D and Gemini-R Formats

LNP-encapsulated forms of D- and Gemini-R were prepared by mixing 5 μg of saDNA or saRNA with 18 μL of Genesome lipid solution (DOTAP:Chol:DOPE in a 1:0.75:0.5 ratio; Encapsula Nano Science, GEN-7036) in a 1:2 volume ratio at room temperature as described by the manufacturer. LNP-protected nucleic acids were kept on ice until ready for injection.

HEK293 Cell Culture and Transfection with Gemini

HEK293 cells (ATCC; CRL-1573) were cultured in Dulbecco's Modified Eagle Medium (DMEM; Gibco, 11965-092) containing 10% Fetal Bovine Serum (FBS; Gibco, A3160702) and penicillin/streptomycin. Cells were seeded at a density of 5*105 cells per well in a 6-well plate one day prior to transfection. Transfection was performed with ˜2.5 μg of either D- or Gemini-R according to the protocol for Lipofectamine™ 3000 (ThermoFisher Scientific, L3000001).

Negative (−) Strand mRNA Detection

HEK293 cells were harvested 72 hrs post-transfection. Total RNA was extracted using the PureLink™ RNA Mini Kit (Ambion, 12183025) and its integrity was checked on a 0.8% agarose gel. Thereafter, total RNA was treated with amplification grade DNase I (Invitrogen, 18068015) to remove any residual DNA, followed by first strand cDNA synthesis using either a NSP4 gene-specific forward primer with a random nucleotide tag sequence (5′-cggtcatggtggcgaataaGCGGCCTTTAATGTGGAATG-3; SEQ ID NO:1) or without any primer according to the SuperScript™ III Reverse Transcriptase protocol (Invitrogen, 18080044). cDNA synthesis was then completed followed by a PCR using an eGFP gene-specific reverse primer (5′-CACCTTGATGCCGTTCTTCT-3′; SEQ ID NO:2_and the random nucleotide tag-specific forward primer (5′-cggtcatggtggcgaataa-3′; SEQ ID NO:3) to produce a 1.9 kb band which would be an indication of negative RNA strand. A nested PCR using the forward primer, 5′-CCGAGAGCTGGTTAGGAGATTA-3′ (SEQ ID and NO: 4), reverse primer, 5′-GCTTGTCGGCCATGATATAGA-3′ (SEQ ID NO:5) on the first PCR product was then performed to amplify cDNA with a band size of 1.4 kb to verify the correct target sequence (see FIG. 2A). Parameters used for both PCRs: 94° C. for 30 seconds, 56° C. for 30 seconds, 72° C. for 30 seconds, for 28 cycles.

### Flow Cytometry Sample Preparation

HEK293 Cells were transfected with either: (1) Gemini-D expressing SARS-CoV-2 spike protein, or (2) a non-self-amplifying DNA plasmid control expressing SARS-CoV-2 spike (see FIG. 2A-ii) or LNP-B.1.617.2 (Delta) spike protein variant of the SARS-CoV-2 virus mRNA or the conventional LNP-encapsulated Pseudouridine substitute mRNAs encoding the B.1.617.2 (Delta) spike protein variant of the SARS-CoV-2 virus. Cells were harvested at 2- and 6-days post-transfection as were the FACS samples and resuspended in FACS buffer at a concentration of 1.0×10⁶ cells/mL. Subsequently, cells were incubated with FACS buffer for 30 minutes at 4° C. cells for Fc blocking, centrifuged (1200 RPM for 4 minutes at 4° C.), and then stained with an anti-receptor-binding domain (RBD) of SARS-CoV-2 spike antibody conjugated to Alexa Fluor 647 (1:100; invitrogen, 51-6490-82) for 30 minutes at 4° C. away from light. Cells were then washed in FACS buffer (1200 RPM for 4 minutes at 4° C.) and resuspended in FACS buffer (500 μL/tube). They were prepared similarly to the FACS samples in the following section, the data was acquired using BD Cytoflex flow cytometer.

### Fluorescence-Activated Cell Sorting (FACS) Sample Preparation

HEK293 Cells were transfected with either a: (1) Gemini-D expressing eGFP, (2) Gemini-R expressing eGFP, or (3) LNP-encapsulated Pseudouridine substitute mRNAs encoding eGFP (Cat #PM-LNP-21) mRNA. Two days post-transfection, cells were sorted for eGFP expression. Cells were then harvested at 14- and 28-days post-transfection using Cellstripper® (Corning, 25-056-CI), counted using a TC20 Automated Cell Counter (Bio-Rad, 1450102), and resuspended in FACS buffer (1× phosphate buffered saline (PBS) with 2% FBS and 2% normal rabbit serum) at a concentration of 1.0×10⁶ cells/mL. Subsequently, cells were incubated with FACS buffer for 30 minutes at 4° C. cells for Fc blocking, centrifuged (1200 RPM for 4 minutes at 4° C.), and resuspended in FACS buffer (500 μL/tube). Data was acquired using a BD Cytoflex flow cytometer.

### Western Blot

HEK293 cells were transfected with either: (1) D- or Gemini-R expressing SARS-CoV-2 spike (2) D- or LNP-encapsulated Pseudouridine substitute mRNAs encoding eGFP (Cat #PM-LNP-21) or LNP-B.1.617.2 (Delta) spike protein variant of the SARS-CoV-2 virus (Cat #PM-LNP-12) mRNA. Cells were harvested 72 hours post-transfection, lysed in 2× sample buffer supplemented with 5% β-Mercaptoethanol (BME; Bio-Rad, 1610710), and heated at 90° C. for 10 minutes. Subsequently, samples were treated with Benzonase nuclease (Sigma, E1014) for 3 hours to remove nucleic acids. A total of 30 μg of protein per well was loaded onto a 4-15% precast SDS-PAGE gel (Bio-Rad, 4561083). SDS-PAGE running conditions are as follows: 75V for 20 minutes, then 120V for 2 hours. Protein was transferred to a nitrocellulose membrane using 75V for 3 hours. Subsequently, the membrane was washed in 0.1% Tween-20 in PBS (PBST) followed by blocking with 5% skim milk in 1% PBST for 2 hours. Primary antibodies for ALFA Tag (Nano-tech, N1581) and eGFP (UBC AbLab, 21-0024-01) were diluted 1:5000 and incubated with the membrane at 4° C. overnight. The membrane was then treated with three 10 minutes washes with 0.1% PBST. Anti-rabbit (ThermoFisher Scientific, 31460) and anti-mouse (Abcam, AB205719) secondary antibodies conjugated to HRP were diluted 1:5000 and incubated with the membrane at room temperature for 1 hour followed by three 10-minute washes of 0.1% PBST. Subsequent signal detection was conducted on a ChemiDoc Imaging System (Bio-Rad).

Qualitative Determination of eGFP Expression in Injected Mouse Leg Muscles

Two groups of 6-12-week-old K18-hACE2 mice were injected with 5 μg of either LNP-Gemini-R or LNP-Gemini-D expressing eGFP, by intramuscular injection into the caudal thigh muscle. For each vaccine group, mice were sacrificed, at 14-, 28- or 42-days post-injection (n=3 per day of sacrifice). Thigh muscles were excised and immediately frozen on dry ice in Neg-50™ (Richard-Allen Scientific, Thermo Scientific). Samples were stored at 80° C. until they were sectioned, using a cryostat microtome, and counter stained with DAPI at the Centre for Phenogenomics, University of Toronto. Images were captured at the same facility (FIG. 4) and were subsequently assessed for qualitative expression of native eGFP. Four sections per sample were assessed; the one with highest eGFP intensity was chosen per sample. A visual ‘average’ was ascertained from these images for each time point and a suitable representative image was selected.

The highest eGFP intensity images were used to quantify eGFP mean intensity (FIGS. 4B-FIG. 4D), utilizing ImageJ software to calculate mean intensity and total area for each sample. Weighted mean intensities were calculated for each vaccine group per time point, as the sum of the individual weighted mean eGFP intensities per sample. The individual weighted means were calculated using the following equation:

$$( \frac{{Area}_{sample}}{Total area per time point} ) mean {intensity}_{sample}$$

### Immunization of Mice

K18-hACE2 transgenic mice were purchased from the Jackson Laboratory and maintained in the Centre for Disease Modeling at the University of British Columbia. These experiments were approved by the Animal Care Committee (UBC). Animals were maintained and euthanized under humane conditions in accordance with the guidelines of the Canadian Council on Animal Care. Groups of 15-week-old K18-hACE2 transgenic mice (n=4 per group; Jackson Laboratory, 034860) were immunized with 5 μg of LNP-encapsulated or 50 μg Naked Gemini-D or Gemini-R formats expressing the B. 1.617.2 (Delta) spike protein variant of the SARS-CoV-2 virus or the eGFP or 50 μg Naked Gemini-D or Gemini-R formats expressing B.1.1.529 (Omicron) Spike variant of SARS CoV-2 Spike vaccine ( ). Optimal doses were determined in prior experiments utilizing other Spike constructs. Mice were immunized by intramuscular injection into the right caudal thigh muscle. Blood samples were taken from the left lateral saphenous vein before vaccination at day 1 and day 28 or day 42 post-initial vaccination. During the study, mice were monitored weekly (or more frequently if needed after injections or blood collection) for any behavioural changes or changes to body condition or weight. A humane end point was determined as a 20% overall weight loss or 10% weight loss from previous weight.

### SARS-CoV-2 Spike ELISA Protocol

SARS-CoV-2 super stable trimer spike protein (ACROBiosystems, SPN-C52H9-50UG) for delta strain and SARS-CoV-2 RBD of spike protein (Proteogenix, Strain B1.1.529, PX-COV-P074) for the Omicron strain was diluted to 100 ng/ml and coated onto 96 well plates using coating buffer (0.1 M Carbonate, pH 9.5). After overnight incubation at 4° C., plates were washed four times with washing buffer (0.1% Tween-20 in 1×PBS). Subsequently, plates were blocked with blocking buffer (2% BSA, 0.1% Tween-20 in 1×PBS) overnight at 4° C. then washed five times with washing buffer. Serum samples from mice immunized with the B. 1.617.2 (Delta) spike protein variant of the SARS-CoV-2 virus or the Omicron (B.1.1.529) spike protein was serially diluted 3-fold in blocking buffer from 1:80 to 1:2160. In each well, 100 μL of serum sample dilutions were added and plates were incubated away from light at 37° C. for 1 hour. Plates were then washed four times with washing buffer before incubating with 100 μL Goat anti-mouse HRP-conjugated secondary antibody (Southern Biotech, 1030-05; 1:4000 dilution in blocking buffer) at 37° C. for 1 hour. Plates were finally washed with washing buffer five times before adding 100 μl/well of TMB substrate (ThermoFisher Scientific, 34028) and incubated away from light at room temperature for 20 minutes to allow for colour development. Reaction was stopped by adding 100 μl/well of stopping solution (0.16 N H₂SO₄). Chemiluminiscence of the plates were read using ELISA plate reader at 450 nm. A B. 1.617.2 (Delta) SARS-CoV-2 spike antibody (ACROBiosystems; S1N-S58A1) was used to set up a standard curve which was structured using Graphpad prism (Version 10.0.1) from which the unknown antibody values were interpolated and the results were expressed in ng/ml. For Omicron spike protein, positive serum was made in house by vaccinating mice with Omicron spike protein in alum adjuvant. Quantified antibody from positive control serum was used to set up standard curve using Graphpad prism (Version 10.0.1) from which the unknown antibody values were interpolated and the results expressed in ng/ml. (FIG. 5B).

eGFP ELISA Protocol

GFP protein (Thermofisher Scientific, A42613) was diluted to 100 ng/ml and coated onto 96 well plates using coating buffer (0.1 M Carbonate, pH 9.5). After overnight incubation at 4° C., plates were washed four times with washing buffer (0.1% Tween-20 in 1×PBS). Subsequently, plates were blocked with blocking buffer (2% BSA, 0.1% Tween-20 in 1×PBS) overnight at 4° C. then washed five times with washing buffer. Serum samples from mice immunized with the eGFP protein were serially diluted 3-fold in blocking buffer from 1:80 to 1:2160. In each well, 100 μL of serum sample dilutions were added and plates were incubated away from light at 37° C. for 1 hour. Plates were then washed four times with washing buffer before incubating with 100 UL Goat anti-mouse HRP-conjugated secondary antibody (ThermoFisher Scientific, 31430; 1:4000 dilution in blocking buffer) at 37° C. for 1 hour. Plates were finally washed with washing buffer five times before adding 100 μl/well of TMB substrate (ThermoFisher Scientific, 34028) and incubated away from light at room temperature for 20 minutes to allow for colour development. Reaction was stopped by adding 100 μl/well of stopping solution (0.16 N H₂SO₄). Chemiluminiscence of the plates were read using ELISA plate reader at 450 nm. A GFP antibody (Thermofisher Scientific, GFP-101AP) was used to set up a standard curve which was structured using Graphpad prism (Version 9.4.1) from which the unknown antibody values were interpolated and the results were expressed in ng/ml (FIG. 5C).

### ELISPOT Assay Protocols

### a) Interferon-γ ELISPOT Assay Protocol

Briefly, the protocol is as follows. On day 0, precoated ELISPOT plates (Mabtech #3321-4HST-2) were washed with PBS and blocked with 200 μl of complete medium (RPMI with 20% or 10% FBS). The plates were incubated for 30 minutes at room temperature. After the incubation, medium was removed and cell suspension (3-5×105 cells/well) was added in complete medium (RPMI with 10% FBS). The Spleen cells were allowed to rest overnight. After overnight rest, lipopolysaccharide (2 μg/ml) and antigen presenting cells (APC) were added. APCs such as Dendritic Cells (DC 2.4) were added at 1:10 ratio to the spleen cells, i.e., 3-5×104 cells/well. Finally, SARS Cov-2 overlapping peptides (JPT Peptide Technologies GmbH, Volmerstrasse 5, 12489 Berlin, Germany) were added to the wells at 1 μg/ml concentration. The plates were subsequently incubated in 37° C. humid chamber with 5% CO2 for 48 hours. After 48 hrs of incubation, the plates were washed with PBS. Biotinylated secondary antibody (Mabtech #3321-6) was subsequently added followed by Streptavidin-HRP (Mabtech #3310-9). Finally, TMB substrate was added for spot development. The spots were read using ELISPOT reader. The data was plotted and analyzed using graphpad prism.

### b) TNF-α ELISPOT Assay Protocol

Briefly, the protocol is as follows. On day 0, precoated ELISPOT plates (Immunospot #mTNFap-2M/2) were washed with PBS and cell suspension (3-5×105 cells/well) was added in complete medium (RPMI with 10% FBS). The Spleen cells were allowed to rest overnight. After overnight rest, lipopolysaccharide (2 μg/ml) and antigen presenting cells (APC) were added. APCs such as Dendritic Cells (DC 2.4) were added at 1:10 ratio to the spleen cells, i.e., 3-5×104 cells/well. Finally, SARS Cov-2 overlapping peptides (JPT Peptide Technologies GmbH, Volmerstrasse 5, 12489 Berlin, Germany) were added to the wells at 1 μg/ml concentration. The plates were subsequently incubated in 37° C. humid chamber with 5% CO2 for 24 hours. After 24 hrs of incubation, the plates were washed with PBS. Biotinylated secondary antibody (Immunospot #mTNFap-2M/2) was subsequently added followed by Streptavidin-HRP (Immunospot #mTNFap-2M/2). Finally, Blue Developer solution was added for spot development. The spots were read using ELISPOT reader. The data was plotted and analyzed using graphpad prism.

### c) IL-4 ELISPOT Assay Protocol

Briefly, the protocol is as follows. On day 0, precoated ELISPOT plates (Immunospot #mIL4p-2M/2) were washed with PBS and cell suspension (3-5×105 cells/well) was added in complete medium (RPMI with 10% FBS). The Spleen cells were allowed to rest overnight. After overnight rest, lipopolysaccharide (2 μg/ml) and antigen presenting cells (APC) were added. APCs such as Dendritic Cells (DC 2.4) were added at 1:10 ratio to the spleen cells, i.e., 3-5×104 cells/well. Finally, SARS Cov-2 overlapping peptides (JPT Peptide Technologies GmbH, Volmerstrasse 5, 12489 Berlin, Germany) were added to the wells at 1 μg/ml concentration. The plates were subsequently incubated in 37° C. humid chamber with 5% CO2 for 24 hours. After 24 hrs of incubation, the plates were washed with PBS. Biotinylated secondary antibody (Immunospot #mIL4p-2M/2) was subsequently added followed by Streptavidin-HRP (Immunospot #mIL4p-2M/2). Finally, Blue Developer solution was added for spot development. The spots were read using ELISPOT reader. The data was plotted and analyzed using graphpad prism.

### Measuring Viraemia in Viral Challenged Mice

K18-hACE2 transgenic mice were purchased from the Jackson Laboratory and maintained in the DCM Division of Comparative Medicine CL3, Viral Core & Biobank at the University of Toronto. These experiments were approved by the Animal Care Committee (UofT). Animals were maintained and euthanized under humane conditions in accordance with the guidelines of the Canadian Council on Animal Care. In separate experiments 3 male and 3 female 6-12 week-old K18-hACE2 transgenic mice (Jackson Laboratory, 034860) were used to evaluate viral load in mice vaccinated with 5 μg of LNP-encapsulated Delta (B.1.617.2) spike protein expressed as 5 μg of LNP-encapsulated or Naked Omicron (B.1.1.529) spike protein encoded by saDNA or PBS controls. A second vaccine dose of 5 μg was given 28 days following the initial dose (of 5 μg; both injected intramuscularly) after which virus challenge was presented 14 days later (on Day 42). The LNP-encapsulated Delta (B.1.617.2) spike protein group was challenged a with the Delta (B.1.617.2) strain of coronavirus variants administered nasally. While the LNP-encapsulated Omicron (B.1.1.529) spike protein encoded by saDNA or Naked saDNA was challenged a with the Omicron (B.1.1.529) strain of the SARS CoV-2 coronavirus variant administered nasally. All mice in the Delta (B.1.617.2) strain of coronavirus group were challenged with 1.6×10⁵ viral units/dose of the Delta (B.1.617.2) SARS-CoV-2 virus. The second group consisted of mice vaccinated with a saDNA inoculum containing the Omicron (B.1.1.529) Spike protein and from mice given no inoculum (i.e. unvaccinated). The mice in this group were vaccinated with the 5 μg of inoculum and on the same inoculum schedule as were the mice in the first group. All mice in this group were challenged with 1.0×10⁵ viral units/dose of the Omicron (B.1.1.529) SARS-CoV-2 virus. Serum was collected by cardiac puncture and stored at −20° C. prior to testing.

### SARS CoV-2 Nucleocapsid ELISA Protocol

Nucleocapsid capture antibody (Acrobiosystem, NUN-CH14) was diluted to 4.7 μg/mL and coated onto 96 well plates using coating buffer (0.1 M Carbonate, pH 9.5). After overnight incubation at 4° C., plates were washed four times with washing buffer (0.1% Tween-20 in 1×PBS). Subsequently, plates were blocked with blocking buffer (2% BSA, 0.1% Tween-20 in 1×PBS) overnight at 4° C. On the day of the assay the plates were washed five times with a washing buffer before further steps. Nucleocapsid protein (Acrobiosystems, NUN-C52Hw) was diluted using serial two-fold dilutions with dilutions ranging from 3.2 ng/ml to 0.05 ng/ml for making a standard curve. Serum samples from SARS-Cov-2 virally challenged mice were serially diluted 3-fold in blocking buffer from 1:240 to 1:2160. In each well, 100 μL of serum sample dilutions were added along with Nucleocapsid standards and the plates were incubated away from light at 37° C. for 1 hour. Plates were then washed five times with washing buffer before incubating with 100 μL of biotinylated anti-nucleocapsid secondary capture antibody (Acrobiosystem, AM223; 1 μg/ml dilution in blocking buffer) at 37° C. for 1 hour. After washing the plates five times, the streptavidin-HRP secondary (Jackson ImmunoResearch, 016-030-084, 1:1000 dilution) was added, and plates again incubated at 37° C. for 1 hour. Plates were finally washed with washing buffer five times before adding 100 μl/well of TMB substrate (ThermoFisher Scientific, 34028) and incubated away from light at room temperature for 10 minutes to allow for colour development. Reaction was stopped by adding 100 μl/well of stopping solution (0.16 N H₂SO₄). Chemiluminescence of the plates were read using ELISA plate reader at 450 nm. Standard was set up using serial dilution of nucleocapsid antigen (Acrobiosystems, NUN-C52Hw) was used to set up a standard curve which was used in GraphPad prism (Version 10.0.2) to interpolate concentration of nucleocapsid protein in viral-challenged mice unknown samples and the results were expressed in ng/ml.

### Frequency of Genomic Integration

The frequency of vector integration in the mouse genome was measured by a method previously described (15) (FIG. 5D). Genomic DNA was extracted from the leg muscles injected with either D- or Gemini-R using tissue/cell lysis buffer (10 mM Tris-CI pH8.0, 0.1M NaCl, 10 mM EDTA, 0.5% SDS), phenol/chloroform/isoamyl alcohol (Invitrogen, 25:24:1, v/v), additional chloroform extraction and ethanol/Sodium acetate precipitation. DNA was subjected to the neutral-neutral 2D gel which separates the linear mouse genomic DNA from any extrachromosomal DNA in the cells. Briefly, 10 μg of genomic DNA was run in 0.4% agarose at 1 V/cm for 18 hours without ethidium bromide (EtBr), and a second dimensional electrophoresis was run in 1% agarose with EtBr at 5 V/cm for 3.5 hours. The DNA bands were then excised from the gel and purified using QiaxII gel extraction kit (Qiagen, Cat. No. 20021) according to the manufacturer's manual.

The gel extracted genomic DNA was further digested by the AscI restriction enzyme (R0558S, NEB) to make sure all the vaccine DNA was eliminated. The pure genomic DNA was separated from any extrachromosomal vaccine DNA by 2D gel electrophoresis and further digested by the AscI restriction Enzyme (R0558S, NEB) since it cuts only DNA propagated in E. coli cells but not genomic DNA due to the different methylation systems in prokaryotic and eukaryotic cells. As plasmid DNA lacks the origin of replication, it is not replicated by the eukaryotic cell machinery and hence it does not undergo CpG methylation. Using this approach, the remaining DNA should represent the mouse genomic DNA population. Furthermore, since the AscI site is located between NSP4 and eGFP, the remaining Gemini 1.0 DNA after successful digestion with AscI should not contribute to PCR amplification.

To determine the copy number correctly, real time q-PCR was performed using the SensiFAST™ SYBR® Kit according to the manufacturer's instruction (BIO-94050, Bioline) and two pairs of primers (NSP4/eGFP forward primer: 5′-GTGCAAGGCAGTAGAATCAAG-3′ (SEQ ID NO:6), NSP4/EGFP reverse primer: 5′-GATGAACTTCAGGGTCAGCTT-3′ (SEQ ID NO:7) and ABCF1 forward primer: 5′-GCCGTCATCTGGCTCAATAA-3′ (SEQ ID NO:8) and ABCF1 reverse primer: 5′-CCTGCTTCTCGTACTGCTTTAG-3′ (SEQ ID NO:9). Each sample was conducted in triplicates and were PCR amplified on an Applied Biosystem 7600; the Ct values from all the samples were analyzed for the expression of eGFP and a single copy endogenous gene, ABCF1.

To calculate the integration frequency accurately, the following rationale was considered. One microgram of genomic DNA has the total of genomic DNA from 166,666 cells/0.5 genomes because the average yield from the single cell is 6 pg (35). Ct values from the AscI treated samples was chosen to the calculate the copy number of Gemini 1.0 DNA spanning from NSP to eGFP in comparison to the Ct value from the standard curved created from serially diluted vaccine DNA.

### Statistical Analysis

Vaccine data was first analysed for significant outliers in Graphpad prism (Version 9.4.1) using Grubbe's test. This data was then analysed using Psych package in RStudio (R version 4.2.0). The resulting summary statistics were used to assess skewness and kurtosis of data distribution. Shapiro-Wilk and Kolmogorov-Smirnoff tests were performed in R to measure the parameters of normal distributions. Normally distributed data was subjected to t test (for two groups) (FIG. 3D, top and bottom panels) or Analysis of Variance (ANOVA) statistical test (both using the Graphpad Prism software, version 9.4.1) along with Tukey's and Dunnett's post-hoc tests to test the differences between more than two groups (FIG. 5B, 5C). Each point on the figure denotes individual animal in the experiment. p-values less than 0.05 were considered significant using 95% confidence intervals.

Sequences

Gemini-Delta (Bold sequences indicate the Delta insert) (SEQ ID NO: 10)
AACGGCTCGTAACATAGGCCTATGCAGCTCTGACGTTATGGAGCGGTCACGTAGAGGGATGTCC
ATTCTTAGAAAGAAGTATTTGAAACCATCCAACAATGTTCTATTCTCTGTTGGCTCGACCATCTAC
CACGAGAAGAGGGACTTACTGAGGAGCTGGCACCTGCCGTCTGTATTTCACTTACGTGGCAAGC
AAAATTACACATGTCGGTGTGAGACTATAGTTAGTTGCGACGGGTACGTCGTTAAAAGAATAGCT
ATCAGTCCAGGCCTGTATGGGAAGCCTTCAGGCTATGCTGCTACGATGCACCGCGAGGGATTCT
TGTGCTGCAAAGTGACAGACACATTGAACGGGGAGAGGGTCTCTTTTCCCGTGTGCACGTATGT
GCCAGCTACATTGTGTGACCAAATGACTGGCATACTGGCAACAGATGTCAGTGCGGACGACGCG
CAAAAACTGCTGGTTGGGCTCAACCAGCGTATAGTCGTCAACGGTCGCACCCAGAGAAACACCA
ATACCATGAAAAATTACCTTTTGCCCGTAGTGGCCCAGGCATTTGCTAGGTGGGCAAAGGAATAT
AAGGAAGATCAAGAAGATGAAAGGCCACTAGGACTACGAGATAGACAGTTAGTCATGGGGTGTT
GTTGGGCTTTTAGAAGGCACAAGATAACATCTATTTATAAGCGCCCGGATACCCAAACCATCATC
AAAGTGAACAGCGATTTCCACTCATTCGTGCTGCCCAGGATAGGCAGTAACACATTGGAGATCG
GGCTGAGAACAAGAATCAGGAAAATGTTAGAGGAGCACAAGGAGCCGTCACCTCTCATTACCGC
CGAGGACGTACAAGAAGCTAAGTGCGCAGCCGATGAGGCTAAGGAGGTGCGTGAAGCCGAGGA
GTTGCGCGCAGCTCTACCACCTTTGGCAGCTGATGTTGAGGAGCCCACTCTGGAGGCAGACGT
CGACTTGATGTTACAAGAGGCTGGGGCCGGCTCAGTGGAGACACCTCGTGGCTTGATAAAGGTT
ACCAGCTACGATGGCGAGGACAAGATCGGCTCTTACGCTGTGCTTTCTCCGCAGGCTGTACTCA
AGAGTGAAAAATTATCTTGCATCCACCCTCTCGCTGAACAAGTCATAGTGATAACACACTCTGGC
CGAAAAGGGCGTTATGCCGTGGAACCATACCATGGTAAAGTAGTGGTGCCAGAGGGACATGCAA
TACCCGTCCAGGACTTTCAAGCTCTGAGTGAAAGTGCCACCATTGTGTACAACGAACGTGAGTTC
GTAAACAGGTACCTGCACCATATTGCCACACATGGAGGAGCGCTGAACACTGATGAAGAATATTA
CAAAACTGTCAAGCCCAGCGAGCACGACGGCGAATACCTGTACGACATCGACAGGAAACAGTGC
GTCAAGAAAGAACTAGTCACTGGGCTAGGGCTCACAGGCGAGCTGGTGGATCCTCCCTTCCATG
AATTCGCCTACGAGAGTCTGAGAACACGACCAGCCGCTCCTTACCAAGTACCAACCATAGGGGT
GTATGGCGTGCCAGGATCAGGCAAGTCTGGCATCATTAAAAGCGCAGTCACCAAAAAAGATCTA
GTGGTGAGCGCCAAGAAAGAAAACTGTGCAGAAATTATAAGGGACGTCAAGAAAATGAAAGGGC
TGGACGTCAATGCCAGAACTGTGGACTCAGTGCTCTTGAATGGATGCAAACACCCCGTAGAGAC
CCTGTATATTGACGAAGCTTTTGCTTGTCATGCAGGTACTCTCAGAGCGCTCATAGCCATTATAA
GACCTAAAAAGGCAGTGCTCTGCGGGGATCCCAAACAGTGCGGTTTTTTTAACATGATGTGCCT
GAAAGTGCATTTTAACCACGAGATTTGCACACAAGTCTTCCACAAAAGCATCTCTCGCCGTTGCA
CTAAATCTGTGACTTCGGTCGTCTCAACCTTGTTTTACGACAAAAAAATGAGAACGACGAATCCG
AAAGAGACTAAGATTGTGATTGACACTACCGGCAGTACCAAACCTAAGCAGGACGATCTCATTCT
CACTTGTTTCAGAGGGTGGGTGAAGCAGTTGCAAATAGATTACAAAGGCAACGAAATAATGACG
GCAGCTGCCTCTCAAGGGCTGACCCGTAAAGGTGTGTATGCCGTTCGGTACAAGGTGAATGAAA
ATCCTCTGTACGCACCCACCTCAGAACATGTGAACGTCCTACTGACCCGCACGGAGGACCGCAT
CGTGTGGAAAACACTAGCCGGCGACCCATGGATAAAAACACTGACTGCCAAGTACCCTGGGAAT
TTCACTGCCACGATAGAGGAGTGGCAAGCAGAGCATGATGCCATCATGAGGCACATCTTGGAGA
GACCGGACCCTACCGACGTCTTCCAGAATAAGGCAAACGTGTGTTGGGCCAAGGCTTTAGTGCC
GGTGCTGAAGACCGCTGGCATAGACATGACCACTGAACAATGGAACACTGTGGATTATTTTGAAA
CGGACAAAGCTCACTCAGCAGAGATAGTATTGAACCAACTATGCGTGAGGTTCTTTGGACTCGAT
CTGGACTCCGGTCTATTTTCTGCACCCACTGTTCCGTTATCCATTAGGAATAATCACTGGGATAA
CTCCCCGTCGCCTAACATGTACGGGCTGAATAAAGAAGTGGTCCGTCAGCTCTCTCGCAGGTAC
CCACAACTGCCTCGGGCAGTTGCCACTGGAAGAGTCTATGACATGAACACTGGTACACTGCGCA
ATTATGATCCGCGCATAAACCTAGTACCTGTAAACAGAAGACTGCCTCATGCTTTAGTCCTCCAC
CATAATGAACACCCACAGAGTGACTTTTCTTCATTCGTCAGCAAATTGAAGGGCAGAACTGTCCT
GGTGGTCGGGGAAAAGTTGTCCGTCCCAGGCAAAATGGTTGACTGGTTGTCAGACCGGCCTGA
GGCTACCTTCAGAGCTCGGCTGGATTTAGGCATCCCAGGTGATGTGCCCAAATATGACATAATAT
TTGTTAATGTGAGGACCCCATATAAATACCATCACTATCAGCAGTGTGAAGACCATGCCATTAAG
CTTAGCATGTTGACCAAGAAAGCTTGTCTGCATCTGAATCCCGGCGGAACCTGTGTCAGCATAG
GTTATGGTTACGCTGACAGGGCCAGCGAAAGCATCATTGGTGCTATAGCGCGGCAGTTCAAGTT
TTCCCGGGTATGCAAACCGAAATCCTCACTTGAAGAGACGGAAGTTCTGTTTGTATTCATTGGGT
ACGATCGCAAGGCCCGTACGCACAATTCTTACAAGCTTTCATCAACCTTGACCAACATTTATACA
GGTTCCAGACTCCACGAAGCCGGATGTGCACCCTCATATCATGTGGTGCGAGGGGATATTGCCA
CGGCCACCGAAGGAGTGATTATAAATGCTGCTAACAGCAAAGGACAACCTGGCGGAGGGGTGT
GCGGAGCGCTGTATAAGAAATTCCCGGAAAGCTTCGATTTACAGCCGATCGAAGTAGGAAAAGC
GCGACTGGTCAAAGGTGCAGCTAAACATATCATTCATGCCGTAGGACCAAACTTCAACAAAGTTT
CGGAGGTTGAAGGTGACAAACAGTTGGCAGAGGCTTATGAGTCCATCGCTAAGATTGTCAACGA
TAACAATTACAAGTCAGTAGCGATTCCACTGTTGTCCACCGGCATCTTTTCCGGGAACAAAGATC
GACTAACCCAATCATTGAACCATTTGCTGACAGCTTTAGACACCACTGATGCAGATGTAGCCATA
TACTGCAGGGACAAGAAATGGGAAATGACTCTCAAGGAAGCAGTGGCTAGGAGAGAAGCAGTG
GAGGAGATATGCATATCCGACGACTCTTCAGTGACAGAACCTGATGCAGAGCTGGTGAGGGTGC
ATCCGAAGAGTTCTTTGGCTGGAAGGAAGGGCTACAGCACAAGCGATGGCAAAACTTTCTCATAT
TTGGAAGGGACCAAGTTTCACCAGGCGGCCAAGGATATAGCAGAAATTAATGCCATGTGGCCCG
TTGCAACGGAGGCCAATGAGCAGGTATGCATGTATATCCTCGGAGAAAGCATGAGCAGTATTAG
GTCGAAATGCCCCGTCGAAGAGTCGGAAGCCTCCACACCACCTAGCACGCTGCCTTGCTTGTGC
ATCCATGCCATGACTCCAGAAAGAGTACAGCGCCTAAAAGCCTCACGTCCAGAACAAATTACTGT
GTGCTCATCCTTTCCATTGCCGAAGTATAGAATCACTGGTGTGCAGAAGATCCAATGCTCCCAGC
CTATATTGTTCTCACCGAAAGTGCCTGCGTATATTCATCCAAGGAAGTATCTCGTGGAAACACCA
CCGGTAGACGAGACTCCGGAGCCATCGGCAGAGAACCAATCCACAGAGGGGACACCTGAACAA
CCACCACTTATAACCGAGGATGAGACCAGGACTAGAACGCCTGAGCCGATCATCATCGAAGAGG
AAGAAGAGGATAGCATAAGTTTGCTGTCAGATGGCCCGACCCACCAGGTGCTGCAAGTCGAGGC
AGACATTCACGGGCCGCCCTCTGTATCTAGCTCATCCTGGTCCATTCCTCATGCATCCGACTTTG
ATGTGGACAGTTTATCCATACTTGACACCCTGGAGGGAGCTAGCGTGACCAGCGGGGCAACGTC
AGCCGAGACTAACTCTTACTTCGCAAAGAGTATGGAGTTTCTGGCGCGACCGGTGCCTGCGCCT
CGAACAGTATTCAGGAACCCTCCACATCCCGCTCCGCGCACAAGAACACCGTCACTTGCACCCA
GCAGGGCCTGCTCGAGAACCAGCCTAGTTTCCACCCCGCCAGGCGTGAATAGGGTGATCACTA
GAGAGGAGCTCGAGGCGCTTACCCCGTCACGCACTCCTAGCAGGTCGGTCTCGAGAACCAGCC
TGGTCTCCAACCCGCCAGGCGTAAATAGGGTGATTACAAGAGAGGAGTTTGAGGCGTTCGTAGC
ACAACAACAATGACGGTTTGATGCGGGTGCATACATCTTTTCCTCCGACACCGGTCAAGGGCATT
TACAACAAAAATCAGTAAGGCAAACGGTGCTATCCGAAGTGGTGTTGGAGAGGACCGAATTGGA
GATTTCGTATGCCCCGCGCCTCGACCAAGAAAAAGAAGAATTACTACGCAAGAAATTACAGTTAA
ATCCCACACCTGCTAACAGAAGCAGATACCAGTCCAGGAAGGTGGAGAACATGAAAGCCATAAC
AGCTAGACGTATTCTGCAAGGCCTAGGGCATTATTTGAAGGCAGAAGGAAAAGTGGAGTGCTAC
CGAACCCTGCATCCTGTTCCTTTGTATTCATCTAGTGTGAACCGTGCCTTTTCAAGCCCCAAGGT
CGCAGTGGAAGCCTGTAACGCCATGTTGAAAGAGAACTTTCCGACTGTGGCTTCTTACTGTATTA
TTCCAGAGTACGATGCCTATTTGGACATGGTTGACGGAGCTTCATGCTGCTTAGACACTGCCAGT
TTTTGCCCTGCAAAGCTGCGCAGCTTTCCAAAGAAACACTCCTATTTGGAACCCACAATACGATC
GGCAGTGCCTTCAGCGATCCAGAACACGCTCCAGAACGTCCTGGCAGCTGCCACAAAAAGAAAT
TGCAATGTCACGCAAATGAGAGAATTGCCCGTATTGGATTCGGCGGCCTTTAATGTGGAATGCTT
CAAGAAATATGCGTGTAATAATGAATATTGGGAAACGTTTAAAGAAAACCCCATCAGGCTTACTGA
AGAAAACGTGGTAAATTACATTACCAAATTAAAAGGACCAAAAGCTGCTGCTCTTTTTGCGAAGAC
ACATAATTTGAATATGTTGCAGGACATACCAATGGACAGGTTTGTAATGGACTTAAAGAGAGACG
TGAAAGTGACTCCAGGAACAAAACATACTGAAGAACGGCCCAAGGTACAGGTGATCCAGGCTGC
CGATCCGCTAGCAACAGCGTATCTGTGCGGAATCCACCGAGAGCTGGTTAGGAGATTAAATGCG
GTCCTGCTTCCGAACATTCATACACTGTTTGATATGTCGGCTGAAGACTTTGACGCTATTATAGCC
GAGCACTTCCAGCCTGGGGATTGTGTTCTGGAAACTGACATCGCGTCGTTTGATAAAAGTGAGG
ACGACGCCATGGCTCTGACCGCGTTAATGATTCTGGAAGACTTAGGTGTGGACGCAGAGCTGTT
GACGCTGATTGAGGCGGCTTTCGGCGAAATTTCATCAATACATTTGCCCACTAAAACTAAATTTAA
ATTCGGAGCCATGATGAAATCTGGAATGTTCCTCACACTGTTTGTGAACACAGTCATTAACATTGT
AATCGCAAGCAGAGTGTTGAGAGAACGGCTAACCGGATCACCATGTGCAGCATTCATTGGAGAT
GACAATATCGTGAAAGGAGTCAAATCGGACAAATTAATGGCAGACAGGTGCGCCACCTGGTTGA
ATATGGAAGTCAAGATTATAGATGCTGTGGTGGGCGAGAAAGCGCCTTATTTCTGTGGAGGGTTT
ATTTTGTGTGACTCCGTGACCGGCACAGCGTGCCGTGTGGCAGACCCCCTAAAAAGGCTGTTTA
AGCTTGGCAAACCTCTGGCAGCAGACGATGAACATGATGATGACAGGAGAAGGGCATTGCATGA
AGAGTCAACACGCTGGAACCGAGTGGGTATTCTTTCAGAGCTGTGCAAGGCAGTAGAATCAAGG
TATGAAACCGTAGGAACTTCCATCATAGTTATGGCCATGACTACTCTAGCTAGCAGTGTTAAATCA
TTCAGCTACCTGAGAGGGGCCCCTATAACTCTCTACGGCTAACCTGAATGGACTACGACATAGTC
TAGTCCGCCAAGTCTGTTTAAACAGCATAT
GGCGCGCCTAAACGAACGCCACCATGTTCGTATTTCTCGTCCTCCTCCCACTTGTTTCTAGTCA
GTGTGTTAATCTTACAACCAGAACTCAATTACCCCCTGCATACACTAATTCTTTCACACGTGGT
GTTTATTACCCTGACAAAGTTTTCAGATCCTCAGTTTTACATTCAACTCAGGACTTGTTCTTACC
TTTCTTTTCCAATGTTACTTGGTTCCATGCTATACATGTCTCTGGGACCAATGGTACTAAGAGGT
TTGATAACCCTGTCCTACCATTTAATGATGGTGTTTATTTTGCTTCCATTGAGAAGTCTAACATA
ATAAGAGGCTGGATTTTTGGTACTACTTTAGATTCGAAGACCCAGTCCCTACTTATTGTTAATAA
CGCTACTAATGTTGTTATTAAAGTCTGTGAATTTCAATTTTGTAATGATCCATTTTTGGGTGTTTA
TTACCACAAAAACAACAAAAGTTGGATGAAAAGTGAGTTCAGAGTTTATTCTAGTGCGAATAAT
TGCACTTTTGAATATGTCTCTCAGCCTTTTCTTATGGACCTTGAAGGAAAACAGGGTAATTTCAA
AAATCTTAGGGAATTTGTGTTTAAGAATATTGATGGTTATTTTAAAATATATTCTAAGCACACGC
CTATTAATTTAGTGCGTGATCTCCCTCAGGGTTTTTCGGCTTTAGAACCATTGGTAGATTTGCCA
ATAGGTATTAACATCACTAGGTTTCAAACTTTACTTGCTTTACATAGAAGTTATTTGACTCCTGG
TGATTCTTCTTCAGGTTGGACAGCTGGTGCTGCAGCTTATTATGTGGGTTATCTTCAACCTAGG
ACTTTTCTATTAAAATATAATGAAAATGGAACCATTACAGATGCTGTAGACTGTGCACTTGACC
CTCTCTCAGAAACAAAGTGTACGTTGAAATCCTTCACTGTAGAAAAAGGAATCTATCAAACTTC
TAACTTTAGAGTCCAACCAACAGAATCTATTGTTAGATTTCCTAATATTACAAACTTGTGCCCTT
TTGGTGAAGTTTTTAACGCCACCAGATTTGCATCTGTTTATGCTTGGAACAGGAAGAGAATCAG
CAACTGTGTTGCTGATTATTCTGTCCTATATAATTCCGCATCATTTTCCACTTTTAAGTGTTATGG
AGTGTCTCCTACTAAATTAAATGATCTCTGCTTTACTAATGTCTATGCAGATTCATTTGTAATTA
GAGGTGATGAAGTCAGACAAATCGCTCCAGGGCAAACTGGAAAGATTGCTGATTATAATTATA
AATTACCAGATGATTTTACAGGCTGCGTTATAGCTTGGAATTCTAACAAACTTGATTCTAAGGTT
GGTGGTAATTATAATTACCGCTATAGATTGTTTAGGAAGTCTAATCTCAAACCTTTTGAGAGAG
ATATTTCAACTGAAATCTATCAGGCCGGTAGCACACCTTGTAATGGTGTTCAGGGTTTTAATTG
TTACTTTCCTTTACAATCATATGGTTTCCAACCCACTTATGGTGTTGGTTACCAACCATACAGAG
TAGTAGTACTTTCTTTTGAACTTCTACATGCACCAGCAACTGTTTGTGGACCTAAAAAGTCTACT
AATTTGGTTAAAAACAAATGTGTCAATTTCAACTTCAATGGTTTAACAGGCACAGGTGTTCTTAC
TGAGTCTAACAAAAAGTTTCTGCCTTTCCAACAATTTGGCAGAGACATTGATGACACTACTGAT
GCTGTCCGTGATCCACAGACACTTGAGATTCTTGACATTACACCATGTTCTTTTGGTGGTGTCA
GTGTTATAACACCAGGAACAAATACTTCTAACCAGGTTGCTGTTCTTTATCAGGGTGTTAACTG
CACAGAAGTCCCTGTTGCTATTCATGCAGATCAACTTACTCCTACTTGGCGTGTTTATTCTACAG
GTTCTAATGTTTTTCAAACACGTGCAGGCTGTTTAATAGGGGCTGAACATGTCAACAACTCATA
TGAGTGTGACATACCCATTGGTGCAGGTATATGCGCTAGTTATCAGACTCAGACTAATTCTAGT
GTAGCTAGTCAATCCATCATTGCCTACACTATGTCACTTGGTGCAGAAAATTCAGTTGCTTACT
CTAATAACTCTATTGCCATACCCATTAATTTTACTATTAGTGTTACCACAGAAATTCTACCAGTG
TCTATGACCAAGACATCAGTAGATTGTACAATGTACATTTGTGGTGATTCAACTGAATGCAGCA
ATCTTTTGTTGCAATATGGCAGTTTTTGTACACAATTAAACCGTGCTTTAACTGGAATAGCTGTT
GAACAAGACAAAAACACCCAAGAAGTTTTTGCACAAGTCAAACAAATTTACAAAACACCACCA
ATTAAAGATTTTGGTGGTTTTAATTTTTCACAAATATTACCAGATCCATCAAAACCAAGCAAGAG
GTCATTTATTGAAGATCTACTTTTCAACAAAGTGACACTTGCAGATGCTGGCTTCATCAAACAA
TATGGTGATTGCCTTGGTGATATTGCTGCTAGAGACCTCATTTGTGCACAAAAGTTTAACGGCC
TTACTGTTTTGCCACCTTTGCTCACAGATGAAATGATTGCTCAATACACTTCTGCACTGTTAGCG
GGTACAATCACTTCTGGTTGGACCTTTGGTGCAGGTGCTGCATTACAAATACCATTTGCTATGC
AAATGGCTTATAGGTTTAATGGTATTGGAGTTACACAGAATGTTCTCTATGAGAACCAAAAATT
GATTGCCAACCAATTTAATAGTGCTATTGGCAAAATTCAAGACTCACTTTCTTCCACAGCAAGT
GCACTTGGAAAACTTCAAGATGTGGTCAACCAAAATGCACAAGCTTTAAACACGCTTGTTAAA
CAACTTAGCTCCAATTTTGGTGCAATTTCAAGTGTTTTAAATGATATCCTTGCACGTCTTGACAA
AGTTGAGGCTGAAGTGCAAATTGATAGGTTGATCACAGGCAGACTTCAAAGTTTGCAGACATA
TGTGACTCAACAATTAATTAGAGCTGCAGAAATCAGAGCTTCTGCTAATCTTGCTGCTACTAAA
ATGTCAGAGTGTGTACTTGGACAATCAAAAAGAGTTGATTTTTGTGGAAAGGGCTATCATCTTA
TGTCCTTCCCTCAGTCAGCACCTCATGGTGTAGTCTTCTTGCATGTGACTTATGTCCCTGCACA
CGAAAAGAACTTCACAACTGCTCCTGCCATTTGTCATGATGGAAAAGCACACTTTCCTCGTGAA
GGTGTCTTTGTTTCAAATGGCACACACTGGTTTGTAACACAAAGGAATTTTTATGAACCACAAA
TCATTACTACACACAACACATTTGTGTCTGGTAACTGTGATGTTGTAATAGGAATTGTCAACAA
CACAGTTTATGATCCTTTGCAACCTGAATTAGACTCATTCAAGGAGGAGTTAGATAAATATTTTA
AGAATCATACATCACCAGATGTTGATTTAGGTGACATCTCTGGCATTAATGCTTCAGTTGTAAA
CATTCAAAAAGAAATTGACCGCCTCAATGAGGTTGCCAAGAATTTAAATGAATCTCTCATCGAT
CTCCAAGAACTTGGAAAGTATGAGCAGTATATAAAATGGCCATGGTACATCTGGCTGGGTTTTA
TAGCTGGCCTGATCGCAATTGTAATGGTAACTATAATGTTGTGTTGCATGACCTCTTGCTGCAG
CTGTTTGAAGGGATGCTGTTCTTGCGGGAGTTGCTGTAAATTTGATGAGGATGACAGCGAGCC
GGTGTTGAAAGGAGTGAAGCTTCATTATACTTCACGACTGGAGGAAGAACTGCGCCGACGCCT
GACTGAATAATCTAGA
GTGTTTAAACCGACCCGGGCGGCCGCAACTAACTTAAGCTAGCAACGGTTTCCCTCTAGCGGGA
TCAATTCCGCCCCCCCCCCCTAACGTTACTGGCCGAAGCCGCTTGGAATAAGGCCGGTGTGCGT
TTGTCTATATGTTATTTTCCACCATATTGCCGTCTTTTGGCAATGTGAGGGCCCGGAAACCTGGC
CCTGTCTTCTTGACGAGCATTCCTAGGGGTCTTTCCCCTCTCGCCAAAGGAATGCAAGGTCTGTT
GAATGTCGTGAAGGAAGCAGTTCCTCTGGAAGCTTCTTGAAGACAAACAACGTCTGTAGCGACC
CTTTGCAGGCAGCGGAACCCCCCACCTGGCGACAGGTGCCTCTGCGGCCAAAAGCCACGTGTA
TAAGATACACCTGCAAAGGCGGCACAACCCCAGTGCCACGTTGTGAGTTGGATAGTTGTGGAAA
GAGTCAAATGGCTCTCCTCAAGCGTATTCAACAAGGGGCTGAAGGATGCCCAGAAGGTACCCCA
TTGTATGGGATCTGATCTGGGGCCTCGGTGCACATGCTTTACATGTGTTTAGTCGAGGTTAAAAA
AACGTCTAGGCCCCCCGAACCACGGGGACGTGGTTTTCCTTTGAAAAACACGATAATACCATGA
CCGAGTACAAGCCCACGGTGCGCCTCGCCACCCGCGACGACGTCCCCAGGGCCGTACGCACC
CTCGCCGCCGCGTTCGCCGACTACCCCGCCACGCGCCACACCGTCGATCCGGACCGCCACATC
GAGCGGGTCACCGAGCTGCAAGAACTCTTCCTCACGCGCGTCGGGCTCGACATCGGCAAGGTG
TGGGTCGCGGACGACGGCGCCGCGGTGGCGGTCTGGACCACGCCGGAGAGCGTCGAAGCGG
GGGCGGTGTTCGCCGAGATCGGCCCGCGCATGGCCGAGTTGAGCGGTTCCCGGCTGGCCGCG
CAGCAACAGATGGAAGGCCTCCTGGCGCCGCACCGGCCCAAGGAGCCCGCGTGGTTCCTGGC
CACCGTCGGCGTCTCGCCCGACCACCAGGGCAAGGGTCTGGGCAGCGCCGTCGTGCTCCCCG
GAGTGGAGGCGGCCGAGCGCGCCGGGGTGCCCGCCTTCCTGGAGACCTCCGCGCCCCGCAAC
CTCCCCTTCTACGAGCGGCTCGGCTTCACCGTCACCGCCGACGTCGAGGTGCCCGAAGGACCG
CGCACCTGGTGCATGACCCGCAAGCCCGGTGCCTGAGAATTGGCAAGCTGCTTACATAGAACTC
GCGGCGATTGGCATGCCGCCTTAAAATTTTTATTTTATTTTTTCTTTTCTTTTCCGAATCGGATTTT
GTTTTTAATATTTCAAAAAAAAAAAAAAAAAAAAAAAAAACGCGTCGAGGGGAATTAATTCTTGAA
GACGAAAGGGCCAGGTGGCACTTTTCGGGGAAATGTGCGCGGAACCCCTATTTGTTTATTTTTCT
AAATACATTCAAATATGTATCCGCTCATGAGACAATAACCCTGATAAATGCTTCAATAATATTGAAA
AAGGAAGAGTATGAGTATTCAACATTTCCGTGTCGCCCTTATTCCCTTTTTTGCGGCATTTTGCCT
TCCTGTTTTTGCTCACCCAGAAACGCTGGTGAAAGTAAAAGATGCTGAAGATCAGTTGGGTGCAC
GAGTGGGTTACATCGAACTGGATCTCAACAGCGGTAAGATCCTTGAGAGTTTTCGCCCCGAAGA
ACGTTTTCCAATGATGAGCACTTTTAAAGTTCTGCTATGTGGCGCGGTATTATCCCGTGTTGACG
CCGGGCAAGAGCAACTCGGTCGCCGCATACACTATTCTCAGAATGACTTGGTTGAGTACTCACC
AGTCACAGAAAAGCATCTTACGGATGGCATGACAGTAAGAGAATTATGCAGTGCTGCCATAACCA
TGAGTGATAACACTGCGGCCAACTTACTTCTGACAACGATCGGAGGACCGAAGGAGCTAACCGC
TTTTTTGCACAACATGGGGGATCATGTAACTCGCCTTGATCGTTGGGAACCGGAGCTGAATGAAG
CCATACCAAACGACGAGCGTGACACCACGATGCCTGTAGCAATGGCAACAACGTTGCGCAAACT
ATTAACTGGCGAACTACTTACTCTAGCTTCCCGGCAACAATTAATAGACTGGATGGAGGCGGATA
AAGTTGCAGGACCACTTCTGCGCTCGGCCCTTCCGGCTGGCTGGTTTATTGCTGATAAATCTGG
AGCCGGTGAGCGTGGGTCTCGCGGTATCATTGCAGCACTGGGGCCAGATGGTAAGCCCTCCCG
TATCGTAGTTATCTACACGACGGGGAGTCAGGCAACTATGGATGAACGAAATAGACAGATCGCT
GAGATAGGTGCCTCACTGATTAAGCATTGGTAACTGTCAGACCAAGTTTACTCATATATACTTTAG
ATTGATTTAAAACTTCATTTTTAATTTAAAAGGATCTAGGTGAAGATCCTTTTTGATAATCTCATGA
CCAAAATCCCTTAACGTGAGTTTTCGTTCCACTGAGCGTCAGACCCCGTAGAAAAGATCAAAGGA
TCTTCTTGAGATCCTTTTTTTCTGCGCGTAATCTGCTGCTTGCAAACAAAAAAACCACCGCTACCA
GCGGTGGTTTGTTTGCCGGATCAAGAGCTACCAACTCTTTTTCCGAAGGTAACTGGCTTCAGCAG
AGCGCAGATACCAAATACTGTCCTTCTAGTGTAGCCGTAGTTAGGCCACCACTTCAAGAACTCTG
TAGCACCGCCTACATACCTCGCTCTGCTAATCCTGTTACCAGTGGCTGCTGCCAGTGGCGATAA
GTCGTGTCTTACCGGGTTGGACTCAAGACGATAGTTACCGGATAAGGCGCAGCGGTCGGGCTG
AACGGGGGGTTCGTGCACACAGCCCAGCTTGGAGCGAACGACCTACACCGAACTGAGATACCT
ACAGCGTGAGCTATGAGAAAGCGCCACGCTTCCCGAAGGGAGAAAGGCGGACAGGTATCCGGT
AAGCGGCAGGGTCGGAACAGGAGAGCGCACGAGGGAGCTTCCAGGGGGAAACGCCTGGTATC
TTTATAGTCCTGTCGGGTTTCGCCACCTCTGACTTGAGCGTCGATTTTTGTGATGCTCGTCAGGG
GGGCGGAGCCTATGGAAAAACGCCAGCAACGCGAGCTCGCGATCGCTTAATTAACGTTACATAA
CTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGG
TAAACTGCCCACTTGGCAGTACATCAAGTGTATCATATGCCAAGTACGCCCCCTATTGACGTCAA
TGACGGTAAATGGCCCGCCTGGCATTATGCCCAGTACATGACCTTATGGGACTTTCCTACTTGGC
AGTACATCTACGTATTAGTCATCGCTATTACCATGGTGATGCGGTTTTGGCAGTACATCAATGGG
CGTGGATAGCGGTTTGACTCACGGGGATTTCCAAGTCTCCACCCCATTGACGTCAATGGGAGTT
TGTTTTGGCACCAAAATCAACGGGACTTTCCAAAATGTCGTAACAACTCCGCCCCATTGACGCAA
ATGGGCGGTAGGCGTGTACGGTGGGAGGTCTATATAAGCAGAGCTTAATACGACTCACTATAGG
GCCGGCCATGGGCGGCGCATGAGAGAAGCCCAGACCAATTACCTACCCAAAATGGAGAAAGTT
CACGTTGACATCGAGGAAGACAGCCCATTCCTCAGAGCTTTGCAGCGGAGCTTCCCGCAGTTTG
AGGTAGAAGCCAAGCAGGTCACTGATAATGACCATGCTAATGCCAGAGCGTTTTCGCATCTGGC
TTCAAAACTGATCGAAACGGAGGTGGACCCATCCGACACGATCCTTGACATTGGAAGTGCGCCC
GCCCGCAGAATGTATTCTAAGCACAAGTATCATTGTATCTGTCCGATGAGATGTGCGGAAGATCC
GGACAGATTGTATAAGTATGCAACTAAGCTGAAGAAAAACTGTAAGGAAATAACTGATAAGGAAT
TGGACAAGAAAATGAAGGAGCTGGCCGCCGTCATGAGCGACCCTGACCTGGAAACTGAGACTAT
GTGCCTCCACGACGACGAGTCGTGTCGCTACGAAGGGCAAGTCGCTGTTTACCAGGATGTATAC
GCGGTTGACGGACCGACAAGTCTCTATCACCAAGCCAATAAGGGAGTTAGAGTCGCCTACTGGA
TAGGCTTTGACACCACCCCTTTTATGTTTAAGAACTTGGCTGGAGCATATCCATCATACTCTACCA
ACTGGGCCGACGAAACCGTGTT
Gemini-EGFP (Bold Green sequences indicate the EGFP insert) (SEQ ID NO: 11)
AACGGCTCGTAACATAGGCCTATGCAGCTCTGACGTTATGGAGCGGTCACGTAGAGGGATGTCC
ATTCTTAGAAAGAAGTATTTGAAACCATCCAACAATGTTCTATTCTCTGTTGGCTCGACCATCTAC
CACGAGAAGAGGGACTTACTGAGGAGCTGGCACCTGCCGTCTGTATTTCACTTACGTGGCAAGC
AAAATTACACATGTCGGTGTGAGACTATAGTTAGTTGCGACGGGTACGTCGTTAAAAGAATAGCT
ATCAGTCCAGGCCTGTATGGGAAGCCTTCAGGCTATGCTGCTACGATGCACCGCGAGGGATTCT
TGTGCTGCAAAGTGACAGACACATTGAACGGGGAGAGGGTCTCTTTTCCCGTGTGCACGTATGT
GCCAGCTACATTGTGTGACCAAATGACTGGCATACTGGCAACAGATGTCAGTGCGGACGACGCG
CAAAAACTGCTGGTTGGGCTCAACCAGCGTATAGTCGTCAACGGTCGCACCCAGAGAAACACCA
ATACCATGAAAAATTACCTTTTGCCCGTAGTGGCCCAGGCATTTGCTAGGTGGGCAAAGGAATAT
AAGGAAGATCAAGAAGATGAAAGGCCACTAGGACTACGAGATAGACAGTTAGTCATGGGGTGTT
GTTGGGCTTTTAGAAGGCACAAGATAACATCTATTTATAAGCGCCCGGATACCCAAACCATCATC
AAAGTGAACAGCGATTTCCACTCATTCGTGCTGCCCAGGATAGGCAGTAACACATTGGAGATCG
GGCTGAGAACAAGAATCAGGAAAATGTTAGAGGAGCACAAGGAGCCGTCACCTCTCATTACCGC
CGAGGACGTACAAGAAGCTAAGTGCGCAGCCGATGAGGCTAAGGAGGTGCGTGAAGCCGAGGA
GTTGCGCGCAGCTCTACCACCTTTGGCAGCTGATGTTGAGGAGCCCACTCTGGAGGCAGACGT
CGACTTGATGTTACAAGAGGCTGGGGCCGGCTCAGTGGAGACACCTCGTGGCTTGATAAAGGTT
ACCAGCTACGATGGCGAGGACAAGATCGGCTCTTACGCTGTGCTTTCTCCGCAGGCTGTACTCA
AGAGTGAAAAATTATCTTGCATCCACCCTCTCGCTGAACAAGTCATAGTGATAACACACTCTGGC
CGAAAAGGGCGTTATGCCGTGGAACCATACCATGGTAAAGTAGTGGTGCCAGAGGGACATGCAA
TACCCGTCCAGGACTTTCAAGCTCTGAGTGAAAGTGCCACCATTGTGTACAACGAACGTGAGTTC
GTAAACAGGTACCTGCACCATATTGCCACACATGGAGGAGCGCTGAACACTGATGAAGAATATTA
CAAAACTGTCAAGCCCAGCGAGCACGACGGCGAATACCTGTACGACATCGACAGGAAACAGTGC
GTCAAGAAAGAACTAGTCACTGGGCTAGGGCTCACAGGCGAGCTGGTGGATCCTCCCTTCCATG
AATTCGCCTACGAGAGTCTGAGAACACGACCAGCCGCTCCTTACCAAGTACCAACCATAGGGGT
GTATGGCGTGCCAGGATCAGGCAAGTCTGGCATCATTAAAAGCGCAGTCACCAAAAAAGATCTA
GTGGTGAGCGCCAAGAAAGAAAACTGTGCAGAAATTATAAGGGACGTCAAGAAAATGAAAGGGC
TGGACGTCAATGCCAGAACTGTGGACTCAGTGCTCTTGAATGGATGCAAACACCCCGTAGAGAC
CCTGTATATTGACGAAGCTTTTGCTTGTCATGCAGGTACTCTCAGAGCGCTCATAGCCATTATAA
GACCTAAAAAGGCAGTGCTCTGCGGGGATCCCAAACAGTGCGGTTTTTTTAACATGATGTGCCT
GAAAGTGCATTTTAACCACGAGATTTGCACACAAGTCTTCCACAAAAGCATCTCTCGCCGTTGCA
CTAAATCTGTGACTTCGGTCGTCTCAACCTTGTTTTACGACAAAAAAATGAGAACGACGAATCCG
AAAGAGACTAAGATTGTGATTGACACTACCGGCAGTACCAAACCTAAGCAGGACGATCTCATTCT
CACTTGTTTCAGAGGGTGGGTGAAGCAGTTGCAAATAGATTACAAAGGCAACGAAATAATGACG
GCAGCTGCCTCTCAAGGGCTGACCCGTAAAGGTGTGTATGCCGTTCGGTACAAGGTGAATGAAA
ATCCTCTGTACGCACCCACCTCAGAACATGTGAACGTCCTACTGACCCGCACGGAGGACCGCAT
CGTGTGGAAAACACTAGCCGGCGACCCATGGATAAAAACACTGACTGCCAAGTACCCTGGGAAT
TTCACTGCCACGATAGAGGAGTGGCAAGCAGAGCATGATGCCATCATGAGGCACATCTTGGAGA
GACCGGACCCTACCGACGTCTTCCAGAATAAGGCAAACGTGTGTTGGGCCAAGGCTTTAGTGCC
GGTGCTGAAGACCGCTGGCATAGACATGACCACTGAACAATGGAACACTGTGGATTATTTTGAAA
CGGACAAAGCTCACTCAGCAGAGATAGTATTGAACCAACTATGCGTGAGGTTCTTTGGACTCGAT
CTGGACTCCGGTCTATTTTCTGCACCCACTGTTCCGTTATCCATTAGGAATAATCACTGGGATAA
CTCCCCGTCGCCTAACATGTACGGGCTGAATAAAGAAGTGGTCCGTCAGCTCTCTCGCAGGTAC
CCACAACTGCCTCGGGCAGTTGCCACTGGAAGAGTCTATGACATGAACACTGGTACACTGCGCA
ATTATGATCCGCGCATAAACCTAGTACCTGTAAACAGAAGACTGCCTCATGCTTTAGTCCTCCAC
CATAATGAACACCCACAGAGTGACTTTTCTTCATTCGTCAGCAAATTGAAGGGCAGAACTGTCCT
GGTGGTCGGGGAAAAGTTGTCCGTCCCAGGCAAAATGGTTGACTGGTTGTCAGACCGGCCTGA
GGCTACCTTCAGAGCTCGGCTGGATTTAGGCATCCCAGGTGATGTGCCCAAATATGACATAATAT
TTGTTAATGTGAGGACCCCATATAAATACCATCACTATCAGCAGTGTGAAGACCATGCCATTAAG
CTTAGCATGTTGACCAAGAAAGCTTGTCTGCATCTGAATCCCGGCGGAACCTGTGTCAGCATAG
GTTATGGTTACGCTGACAGGGCCAGCGAAAGCATCATTGGTGCTATAGCGCGGCAGTTCAAGTT
TTCCCGGGTATGCAAACCGAAATCCTCACTTGAAGAGACGGAAGTTCTGTTTGTATTCATTGGGT
ACGATCGCAAGGCCCGTACGCACAATTCTTACAAGCTTTCATCAACCTTGACCAACATTTATACA
GGTTCCAGACTCCACGAAGCCGGATGTGCACCCTCATATCATGTGGTGCGAGGGGATATTGCCA
CGGCCACCGAAGGAGTGATTATAAATGCTGCTAACAGCAAAGGACAACCTGGCGGAGGGGTGT
GCGGAGCGCTGTATAAGAAATTCCCGGAAAGCTTCGATTTACAGCCGATCGAAGTAGGAAAAGC
GCGACTGGTCAAAGGTGCAGCTAAACATATCATTCATGCCGTAGGACCAAACTTCAACAAAGTTT
CGGAGGTTGAAGGTGACAAACAGTTGGCAGAGGCTTATGAGTCCATCGCTAAGATTGTCAACGA
TAACAATTACAAGTCAGTAGCGATTCCACTGTTGTCCACCGGCATCTTTTCCGGGAACAAAGATC
GACTAACCCAATCATTGAACCATTTGCTGACAGCTTTAGACACCACTGATGCAGATGTAGCCATA
TACTGCAGGGACAAGAAATGGGAAATGACTCTCAAGGAAGCAGTGGCTAGGAGAGAAGCAGTG
GAGGAGATATGCATATCCGACGACTCTTCAGTGACAGAACCTGATGCAGAGCTGGTGAGGGTGC
ATCCGAAGAGTTCTTTGGCTGGAAGGAAGGGCTACAGCACAAGCGATGGCAAAACTTTCTCATAT
TTGGAAGGGACCAAGTTTCACCAGGCGGCCAAGGATATAGCAGAAATTAATGCCATGTGGCCCG
TTGCAACGGAGGCCAATGAGCAGGTATGCATGTATATCCTCGGAGAAAGCATGAGCAGTATTAG
GTCGAAATGCCCCGTCGAAGAGTCGGAAGCCTCCACACCACCTAGCACGCTGCCTTGCTTGTGC
ATCCATGCCATGACTCCAGAAAGAGTACAGCGCCTAAAAGCCTCACGTCCAGAACAAATTACTGT
GTGCTCATCCTTTCCATTGCCGAAGTATAGAATCACTGGTGTGCAGAAGATCCAATGCTCCCAGC
CTATATTGTTCTCACCGAAAGTGCCTGCGTATATTCATCCAAGGAAGTATCTCGTGGAAACACCA
CCGGTAGACGAGACTCCGGAGCCATCGGCAGAGAACCAATCCACAGAGGGGACACCTGAACAA
CCACCACTTATAACCGAGGATGAGACCAGGACTAGAACGCCTGAGCCGATCATCATCGAAGAGG
AAGAAGAGGATAGCATAAGTTTGCTGTCAGATGGCCCGACCCACCAGGTGCTGCAAGTCGAGGC
AGACATTCACGGGCCGCCCTCTGTATCTAGCTCATCCTGGTCCATTCCTCATGCATCCGACTTTG
ATGTGGACAGTTTATCCATACTTGACACCCTGGAGGGAGCTAGCGTGACCAGCGGGGCAACGTC
AGCCGAGACTAACTCTTACTTCGCAAAGAGTATGGAGTTTCTGGCGCGACCGGTGCCTGCGCCT
CGAACAGTATTCAGGAACCCTCCACATCCCGCTCCGCGCACAAGAACACCGTCACTTGCACCCA
GCAGGGCCTGCTCGAGAACCAGCCTAGTTTCCACCCCGCCAGGCGTGAATAGGGTGATCACTA
GAGAGGAGCTCGAGGCGCTTACCCCGTCACGCACTCCTAGCAGGTCGGTCTCGAGAACCAGCC
TGGTCTCCAACCCGCCAGGCGTAAATAGGGTGATTACAAGAGAGGAGTTTGAGGCGTTCGTAGC
ACAACAACAATGACGGTTTGATGCGGGTGCATACATCTTTTCCTCCGACACCGGTCAAGGGCATT
TACAACAAAAATCAGTAAGGCAAACGGTGCTATCCGAAGTGGTGTTGGAGAGGACCGAATTGGA
GATTTCGTATGCCCCGCGCCTCGACCAAGAAAAAGAAGAATTACTACGCAAGAAATTACAGTTAA
ATCCCACACCTGCTAACAGAAGCAGATACCAGTCCAGGAAGGTGGAGAACATGAAAGCCATAAC
AGCTAGACGTATTCTGCAAGGCCTAGGGCATTATTTGAAGGCAGAAGGAAAAGTGGAGTGCTAC
CGAACCCTGCATCCTGTTCCTTTGTATTCATCTAGTGTGAACCGTGCCTTTTCAAGCCCCAAGGT
CGCAGTGGAAGCCTGTAACGCCATGTTGAAAGAGAACTTTCCGACTGTGGCTTCTTACTGTATTA
TTCCAGAGTACGATGCCTATTTGGACATGGTTGACGGAGCTTCATGCTGCTTAGACACTGCCAGT
TTTTGCCCTGCAAAGCTGCGCAGCTTTCCAAAGAAACACTCCTATTTGGAACCCACAATACGATC
GGCAGTGCCTTCAGCGATCCAGAACACGCTCCAGAACGTCCTGGCAGCTGCCACAAAAAGAAAT
TGCAATGTCACGCAAATGAGAGAATTGCCCGTATTGGATTCGGCGGCCTTTAATGTGGAATGCTT
CAAGAAATATGCGTGTAATAATGAATATTGGGAAACGTTTAAAGAAAACCCCATCAGGCTTACTGA
AGAAAACGTGGTAAATTACATTACCAAATTAAAAGGACCAAAAGCTGCTGCTCTTTTTGCGAAGAC
ACATAATTTGAATATGTTGCAGGACATACCAATGGACAGGTTTGTAATGGACTTAAAGAGAGACG
TGAAAGTGACTCCAGGAACAAAACATACTGAAGAACGGCCCAAGGTACAGGTGATCCAGGCTGC
CGATCCGCTAGCAACAGCGTATCTGTGCGGAATCCACCGAGAGCTGGTTAGGAGATTAAATGCG
GTCCTGCTTCCGAACATTCATACACTGTTTGATATGTCGGCTGAAGACTTTGACGCTATTATAGCC
GAGCACTTCCAGCCTGGGGATTGTGTTCTGGAAACTGACATCGCGTCGTTTGATAAAAGTGAGG
ACGACGCCATGGCTCTGACCGCGTTAATGATTCTGGAAGACTTAGGTGTGGACGCAGAGCTGTT
GACGCTGATTGAGGCGGCTTTCGGCGAAATTTCATCAATACATTTGCCCACTAAAACTAAATTTAA
ATTCGGAGCCATGATGAAATCTGGAATGTTCCTCACACTGTTTGTGAACACAGTCATTAACATTGT
AATCGCAAGCAGAGTGTTGAGAGAACGGCTAACCGGATCACCATGTGCAGCATTCATTGGAGAT
GACAATATCGTGAAAGGAGTCAAATCGGACAAATTAATGGCAGACAGGTGCGCCACCTGGTTGA
ATATGGAAGTCAAGATTATAGATGCTGTGGTGGGCGAGAAAGCGCCTTATTTCTGTGGAGGGTTT
ATTTTGTGTGACTCCGTGACCGGCACAGCGTGCCGTGTGGCAGACCCCCTAAAAAGGCTGTTTA
AGCTTGGCAAACCTCTGGCAGCAGACGATGAACATGATGATGACAGGAGAAGGGCATTGCATGA
AGAGTCAACACGCTGGAACCGAGTGGGTATTCTTTCAGAGCTGTGCAAGGCAGTAGAATCAAGG
TATGAAACCGTAGGAACTTCCATCATAGTTATGGCCATGACTACTCTAGCTAGCAGTGTTAAATCA
TTCAGCTACCTGAGAGGGGCCCCTATAACTCTCTACGGCTAACCTGAATGGACTACGACATAGTC
TAGTCCGCCAAGTCTGTTTAAACAGCATATGGGCGCGCCCTCAGCATCGATTCAATTCGCCACC
ATGGTGAGCAAGGGCGAGGAGCTGTTCACCGGGGTGGTGCCCATCCTGGTCGAGCTGGACGG
CGACGTAAACGGCCACAAGTTCAGCGTGTCCGGCGAGGGCGAGGGCGATGCCACCTACGGC
AAGCTGACCCTGAAGTTCATCTGCACCACCGGCAAGCTGCCCGTGCCCTGGCCCACCCTCGTG
ACCACCCTGACCTACGGCGTGCAGTGCTTCAGCCGCTACCCCGACCACATGAAGCAGCACGA
CTTCTTCAAGTCCGCCATGCCCGAAGGCTACGTCCAGGAGCGCACCATCTTCTTCAAGGACGA
CGGCAACTACAAGACCCGCGCCGAGGTGAAGTTCGAGGGCGACACCCTGGTGAACCGCATCG
AGCTGAAGGGCATCGACTTCAAGGAGGACGGCAACATCCTGGGGCACAAGCTGGAGTACAAC
TACAACAGCCACAACGTCTATATCATGGCCGACAAGCAGAAGAACGGCATCAAGGTGAACTTC
AAGATCCGCCACAACATCGAGGACGGCAGCGTGCAGCTCGCCGACCACTACCAGCAGAACAC
CCCCATCGGCGACGGCCCCGTGCTGCTGCCCGACAACCACTACCTGAGCACCCAGTCCGCCC
TGAGCAAAGACCCCAACGAGAAGCGCGATCACATGGTCCTGCTGGAGTTCGTGACCGCCGCC
GGGATCACTCTCGGCATGGACGAGCTGTACAAGTAGTCTAGAGTGTTTAAACCGACCCGGGCG
GCCGCAACTAACTTAAGCTAGCAACGGTTTCCCTCTAGCGGGATCAATTCCGCCCCCCCCCCCT
AACGTTACTGGCCGAAGCCGCTTGGAATAAGGCCGGTGTGCGTTTGTCTATATGTTATTTTCCAC
CATATTGCCGTCTTTTGGCAATGTGAGGGCCCGGAAACCTGGCCCTGTCTTCTTGACGAGCATT
CCTAGGGGTCTTTCCCCTCTCGCCAAAGGAATGCAAGGTCTGTTGAATGTCGTGAAGGAAGCAG
TTCCTCTGGAAGCTTCTTGAAGACAAACAACGTCTGTAGCGACCCTTTGCAGGCAGCGGAACCC
CCCACCTGGCGACAGGTGCCTCTGCGGCCAAAAGCCACGTGTATAAGATACACCTGCAAAGGC
GGCACAACCCCAGTGCCACGTTGTGAGTTGGATAGTTGTGGAAAGAGTCAAATGGCTCTCCTCA
AGCGTATTCAACAAGGGGCTGAAGGATGCCCAGAAGGTACCCCATTGTATGGGATCTGATCTGG
GGCCTCGGTGCACATGCTTTACATGTGTTTAGTCGAGGTTAAAAAAACGTCTAGGCCCCCCGAA
CCACGGGGACGTGGTTTTCCTTTGAAAAACACGATAATACCATGACCGAGTACAAGCCCACGGT
GCGCCTCGCCACCCGCGACGACGTCCCCAGGGCCGTACGCACCCTCGCCGCCGCGTTCGCCG
ACTACCCCGCCACGCGCCACACCGTCGATCCGGACCGCCACATCGAGCGGGTCACCGAGCTGC
AAGAACTCTTCCTCACGCGCGTCGGGCTCGACATCGGCAAGGTGTGGGTCGCGGACGACGGCG
CCGCGGTGGCGGTCTGGACCACGCCGGAGAGCGTCGAAGCGGGGGCGGTGTTCGCCGAGATC
GGCCCGCGCATGGCCGAGTTGAGCGGTTCCCGGCTGGCCGCGCAGCAACAGATGGAAGGCCT
CCTGGCGCCGCACCGGCCCAAGGAGCCCGCGTGGTTCCTGGCCACCGTCGGCGTCTCGCCCG
ACCACCAGGGCAAGGGTCTGGGCAGCGCCGTCGTGCTCCCCGGAGTGGAGGCGGCCGAGCGC
GCCGGGGTGCCCGCCTTCCTGGAGACCTCCGCGCCCCGCAACCTCCCCTTCTACGAGCGGCTC
GGCTTCACCGTCACCGCCGACGTCGAGGTGCCCGAAGGACCGCGCACCTGGTGCATGACCCG
CAAGCCCGGTGCCTGAGAATTGGCAAGCTGCTTACATAGAACTCGCGGCGATTGGCATGCCGC
CTTAAAATTTTTATTTTATTTTTTCTTTTCTTTTCCGAATCGGATTTTGTTTTTAATATTTCAAAAAAA
AAAAAAAAAAAAAAAAAAACGCGTCGAGGGGAATTAATTCTTGAAGACGAAAGGGCCAGGTGGC
ACTTTTCGGGGAAATGTGCGCGGAACCCCTATTTGTTTATTTTTCTAAATACATTCAAATATGTATC
CGCTCATGAGACAATAACCCTGATAAATGCTTCAATAATATTGAAAAAGGAAGAGTATGAGTATTC
AACATTTCCGTGTCGCCCTTATTCCCTTTTTTGCGGCATTTTGCCTTCCTGTTTTTGCTCACCCAG
AAACGCTGGTGAAAGTAAAAGATGCTGAAGATCAGTTGGGTGCACGAGTGGGTTACATCGAACT
GGATCTCAACAGCGGTAAGATCCTTGAGAGTTTTCGCCCCGAAGAACGTTTTCCAATGATGAGCA
CTTTTAAAGTTCTGCTATGTGGCGCGGTATTATCCCGTGTTGACGCCGGGCAAGAGCAACTCGG
TCGCCGCATACACTATTCTCAGAATGACTTGGTTGAGTACTCACCAGTCACAGAAAAGCATCTTA
CGGATGGCATGACAGTAAGAGAATTATGCAGTGCTGCCATAACCATGAGTGATAACACTGCGGC
CAACTTACTTCTGACAACGATCGGAGGACCGAAGGAGCTAACCGCTTTTTTGCACAACATGGGG
GATCATGTAACTCGCCTTGATCGTTGGGAACCGGAGCTGAATGAAGCCATACCAAACGACGAGC
GTGACACCACGATGCCTGTAGCAATGGCAACAACGTTGCGCAAACTATTAACTGGCGAACTACTT
ACTCTAGCTTCCCGGCAACAATTAATAGACTGGATGGAGGCGGATAAAGTTGCAGGACCACTTCT
GCGCTCGGCCCTTCCGGCTGGCTGGTTTATTGCTGATAAATCTGGAGCCGGTGAGCGTGGGTCT
CGCGGTATCATTGCAGCACTGGGGCCAGATGGTAAGCCCTCCCGTATCGTAGTTATCTACACGA
CGGGGAGTCAGGCAACTATGGATGAACGAAATAGACAGATCGCTGAGATAGGTGCCTCACTGAT
TAAGCATTGGTAACTGTCAGACCAAGTTTACTCATATATACTTTAGATTGATTTAAAACTTCATTTT
TAATTTAAAAGGATCTAGGTGAAGATCCTTTTTGATAATCTCATGACCAAAATCCCTTAACGTGAG
TTTTCGTTCCACTGAGCGTCAGACCCCGTAGAAAAGATCAAAGGATCTTCTTGAGATCCTTTTTTT
CTGCGCGTAATCTGCTGCTTGCAAACAAAAAAACCACCGCTACCAGCGGTGGTTTGTTTGCCGG
ATCAAGAGCTACCAACTCTTTTTCCGAAGGTAACTGGCTTCAGCAGAGCGCAGATACCAAATACT
GTCCTTCTAGTGTAGCCGTAGTTAGGCCACCACTTCAAGAACTCTGTAGCACCGCCTACATACCT
CGCTCTGCTAATCCTGTTACCAGTGGCTGCTGCCAGTGGCGATAAGTCGTGTCTTACCGGGTTG
GACTCAAGACGATAGTTACCGGATAAGGCGCAGCGGTCGGGCTGAACGGGGGGTTCGTGCACA
CAGCCCAGCTTGGAGCGAACGACCTACACCGAACTGAGATACCTACAGCGTGAGCTATGAGAAA
GCGCCACGCTTCCCGAAGGGAGAAAGGCGGACAGGTATCCGGTAAGCGGCAGGGTCGGAACA
GGAGAGCGCACGAGGGAGCTTCCAGGGGGAAACGCCTGGTATCTTTATAGTCCTGTCGGGTTT
CGCCACCTCTGACTTGAGCGTCGATTTTTGTGATGCTCGTCAGGGGGGCGGAGCCTATGGAAAA
ACGCCAGCAACGCGAGCTCGCGATCGCTTAATTAACGTTACATAACTTACGGTAAATGGCCCGC
CTGGCTGACCGCCCAACGACCCCCGCCCATTGACGTCAATAATGACGTATGTTOCCATAGTAAC
GCCAATAGGGACTTTCCATTGACGTCAATGGGTGGAGTATTTACGGTAAACTGCCCACTTGGCA
GTACATCAAGTGTATCATATGCCAAGTACGCCCCCTATTGACGTCAATGACGGTAAATGGCCCGC
CTGGCATTATGCCCAGTACATGACCTTATGGGACTTTCCTACTTGGCAGTACATCTACGTATTAGT
CATCGCTATTACCATGGTGATGCGGTTTTGGCAGTACATCAATGGGCGTGGATAGCGGTTTGACT
CACGGGGATTTCCAAGTCTCCACCCCATTGACGTCAATGGGAGTTTGTTTTGGCACCAAAATCAA
CGGGACTTTCCAAAATGTCGTAACAACTCCGCCCCATTGACGCAAATGGGGGGTAGGCGTGTAC
GGTGGGAGGTCTATATAAGCAGAGCTGGTTTAGTGAACCGTCAGATCCGCTAGTAATACGACTC
ACTATAGGGCCGGCCATGGGCGGCGCATGAGAGAAGCCCAGACCAATTACCTACCCAAAATGG
AGAAAGTTCACGTTGACATCGAGGAAGACAGCCCATTCCTCAGAGCTTTGCAGCGGAGCTTCCC
GCAGTTTGAGGTAGAAGCCAAGCAGGTCACTGATAATGACCATGCTAATGCCAGAGCGTTTTCG
CATCTGGCTTCAAAACTGATCGAAACGGAGGTGGACCCATCCGACACGATCCTTGACATTGGAA
GTGCGCCCGCCCGCAGAATGTATTCTAAGCACAAGTATCATTGTATCTGTCCGATGAGATGTGC
GGAAGATCCGGACAGATTGTATAAGTATGCAACTAAGCTGAAGAAAAACTGTAAGGAAATAACTG
ATAAGGAATTGGACAAGAAAATGAAGGAGCTGGCCGCCGTCATGAGCGACCCTGACCTGGAAAC
TGAGACTATGTGCCTCCACGACGACGAGTCGTGTCGCTACGAAGGGCAAGTCGCTGTTTACCAG
GATGTATACGCGGTTGACGGACCGACAAGTCTCTATCACCAAGCCAATAAGGGAGTTAGAGTCG
CCTACTGGATAGGCTTTGACACCACCCCTTTTATGTTTAAGAACTTGGCTGGAGCATATCCATCAT
ACTCTACCAACTGGGCCGACGAAACCGTGTT
Gemini-Delta_S1 + S2 (Bold sequences indicates Delta_S1 + s2 insert) (SEQ ID NO: 12)
AACGGCTCGTAACATAGGCCTATGCAGCTCTGACGTTATGGAGCGGTCACGTAGAGGGATGTCC
ATTCTTAGAAAGAAGTATTTGAAACCATCCAACAATGTTCTATTCTCTGTTGGCTCGACCATCTAC
CACGAGAAGAGGGACTTACTGAGGAGCTGGCACCTGCCGTCTGTATTTCACTTACGTGGCAAGC
AAAATTACACATGTCGGTGTGAGACTATAGTTAGTTGCGACGGGTACGTCGTTAAAAGAATAGCT
ATCAGTCCAGGCCTGTATGGGAAGCCTTCAGGCTATGCTGCTACGATGCACCGCGAGGGATTCT
TGTGCTGCAAAGTGACAGACACATTGAACGGGGAGAGGGTCTCTTTTCCCGTGTGCACGTATGT
GCCAGCTACATTGTGTGACCAAATGACTGGCATACTGGCAACAGATGTCAGTGCGGACGACGCG
CAAAAACTGCTGGTTGGGCTCAACCAGCGTATAGTCGTCAACGGTCGCACCCAGAGAAACACCA
ATACCATGAAAAATTACCTTTTGCCCGTAGTGGCCCAGGCATTTGCTAGGTGGGCAAAGGAATAT
AAGGAAGATCAAGAAGATGAAAGGCCACTAGGACTACGAGATAGACAGTTAGTCATGGGGTGTT
GTTGGGCTTTTAGAAGGCACAAGATAACATCTATTTATAAGCGCCCGGATACCCAAACCATCATC
AAAGTGAACAGCGATTTCCACTCATTCGTGCTGCCCAGGATAGGCAGTAACACATTGGAGATCG
GGCTGAGAACAAGAATCAGGAAAATGTTAGAGGAGCACAAGGAGCCGTCACCTCTCATTACCGC
CGAGGACGTACAAGAAGCTAAGTGCGCAGCCGATGAGGCTAAGGAGGTGCGTGAAGCCGAGGA
GTTGCGCGCAGCTCTACCACCTTTGGCAGCTGATGTTGAGGAGCCCACTCTGGAGGCAGACGT
CGACTTGATGTTACAAGAGGCTGGGGCCGGCTCAGTGGAGACACCTCGTGGCTTGATAAAGGTT
ACCAGCTACGATGGCGAGGACAAGATCGGCTCTTACGCTGTGCTTTCTCCGCAGGCTGTACTCA
AGAGTGAAAAATTATCTTGCATCCACCCTCTCGCTGAACAAGTCATAGTGATAACACACTCTGGC
CGAAAAGGGCGTTATGCCGTGGAACCATACCATGGTAAAGTAGTGGTGCCAGAGGGACATGCAA
TACCCGTCCAGGACTTTCAAGCTCTGAGTGAAAGTGCCACCATTGTGTACAACGAACGTGAGTTC
GTAAACAGGTACCTGCACCATATTGCCACACATGGAGGAGCGCTGAACACTGATGAAGAATATTA
CAAAACTGTCAAGCCCAGCGAGCACGACGGCGAATACCTGTACGACATCGACAGGAAACAGTGC
GTCAAGAAAGAACTAGTCACTGGGCTAGGGCTCACAGGCGAGCTGGTGGATCCTCCCTTCCATG
AATTCGCCTACGAGAGTCTGAGAACACGACCAGCCGCTCCTTACCAAGTACCAACCATAGGGGT
GTATGGCGTGCCAGGATCAGGCAAGTCTGGCATCATTAAAAGCGCAGTCACCAAAAAAGATCTA
GTGGTGAGCGCCAAGAAAGAAAACTGTGCAGAAATTATAAGGGACGTCAAGAAAATGAAAGGGC
TGGACGTCAATGCCAGAACTGTGGACTCAGTGCTCTTGAATGGATGCAAACACCCCGTAGAGAC
CCTGTATATTGACGAAGCTTTTGCTTGTCATGCAGGTACTCTCAGAGCGCTCATAGCCATTATAA
GACCTAAAAAGGCAGTGCTCTGCGGGGATCCCAAACAGTGCGGTTTTTTTAACATGATGTGCCT
GAAAGTGCATTTTAACCACGAGATTTGCACACAAGTCTTCCACAAAAGCATCTCTCGCCGTTGCA
CTAAATCTGTGACTTCGGTCGTCTCAACCTTGTTTTACGACAAAAAAATGAGAACGACGAATCCG
AAAGAGACTAAGATTGTGATTGACACTACCGGCAGTACCAAACCTAAGCAGGACGATCTCATTCT
CACTTGTTTCAGAGGGTGGGTGAAGCAGTTGCAAATAGATTACAAAGGCAACGAAATAATGACG
GCAGCTGCCTCTCAAGGGCTGACCCGTAAAGGTGTGTATGCCGTTCGGTACAAGGTGAATGAAA
ATCCTCTGTACGCACCCACCTCAGAACATGTGAACGTCCTACTGACCCGCACGGAGGACCGCAT
CGTGTGGAAAACACTAGCCGGCGACCCATGGATAAAAACACTGACTGCCAAGTACCCTGGGAAT
TTCACTGCCACGATAGAGGAGTGGCAAGCAGAGCATGATGCCATCATGAGGCACATCTTGGAGA
GACCGGACCCTACCGACGTCTTCCAGAATAAGGCAAACGTGTGTTGGGCCAAGGCTTTAGTGCC
GGTGCTGAAGACCGCTGGCATAGACATGACCACTGAACAATGGAACACTGTGGATTATTTTGAAA
CGGACAAAGCTCACTCAGCAGAGATAGTATTGAACCAACTATGCGTGAGGTTCTTTGGACTCGAT
CTGGACTCCGGTCTATTTTCTGCACCCACTGTTCCGTTATCCATTAGGAATAATCACTGGGATAA
CTCCCCGTCGCCTAACATGTACGGGCTGAATAAAGAAGTGGTCCGTCAGCTCTCTCGCAGGTAC
CCACAACTGCCTCGGGCAGTTGCCACTGGAAGAGTCTATGACATGAACACTGGTACACTGCGCA
ATTATGATCCGCGCATAAACCTAGTACCTGTAAACAGAAGACTGCCTCATGCTTTAGTCCTCCAC
CATAATGAACACCCACAGAGTGACTTTTCTTCATTCGTCAGCAAATTGAAGGGCAGAACTGTCCT
GGTGGTCGGGGAAAAGTTGTCCGTCCCAGGCAAAATGGTTGACTGGTTGTCAGACCGGCCTGA
GGCTACCTTCAGAGCTCGGCTGGATTTAGGCATCCCAGGTGATGTGCCCAAATATGACATAATAT
TTGTTAATGTGAGGACCCCATATAAATACCATCACTATCAGCAGTGTGAAGACCATGCCATTAAG
CTTAGCATGTTGACCAAGAAAGCTTGTCTGCATCTGAATCCCGGCGGAACCTGTGTCAGCATAG
GTTATGGTTACGCTGACAGGGCCAGCGAAAGCATCATTGGTGCTATAGCGCGGCAGTTCAAGTT
TTCCCGGGTATGCAAACCGAAATCCTCACTTGAAGAGACGGAAGTTCTGTTTGTATTCATTGGGT
ACGATCGCAAGGCCCGTACGCACAATTCTTACAAGCTTTCATCAACCTTGACCAACATTTATACA
GGTTCCAGACTCCACGAAGCCGGATGTGCACCCTCATATCATGTGGTGCGAGGGGATATTGCCA
CGGCCACCGAAGGAGTGATTATAAATGCTGCTAACAGCAAAGGACAACCTGGCGGAGGGGTGT
GCGGAGCGCTGTATAAGAAATTCCCGGAAAGCTTCGATTTACAGCCGATCGAAGTAGGAAAAGC
GCGACTGGTCAAAGGTGCAGCTAAACATATCATTCATGCCGTAGGACCAAACTTCAACAAAGTTT
CGGAGGTTGAAGGTGACAAACAGTTGGCAGAGGCTTATGAGTCCATCGCTAAGATTGTCAACGA
TAACAATTACAAGTCAGTAGCGATTCCACTGTTGTCCACCGGCATCTTTTCCGGGAACAAAGATC
GACTAACCCAATCATTGAACCATTTGCTGACAGCTTTAGACACCACTGATGCAGATGTAGCCATA
TACTGCAGGGACAAGAAATGGGAAATGACTCTCAAGGAAGCAGTGGCTAGGAGAGAAGCAGTG
GAGGAGATATGCATATCCGACGACTCTTCAGTGACAGAACCTGATGCAGAGCTGGTGAGGGTGC
ATCCGAAGAGTTCTTTGGCTGGAAGGAAGGGCTACAGCACAAGCGATGGCAAAACTTTCTCATAT
TTGGAAGGGACCAAGTTTCACCAGGCGGCCAAGGATATAGCAGAAATTAATGCCATGTGGCCCG
TTGCAACGGAGGCCAATGAGCAGGTATGCATGTATATCCTCGGAGAAAGCATGAGCAGTATTAG
GTCGAAATGCCCCGTCGAAGAGTCGGAAGCCTCCACACCACCTAGCACGCTGCCTTGCTTGTGC
ATCCATGCCATGACTCCAGAAAGAGTACAGCGCCTAAAAGCCTCACGTCCAGAACAAATTACTGT
GTGCTCATCCTTTCCATTGCCGAAGTATAGAATCACTGGTGTGCAGAAGATCCAATGCTCCCAGC
CTATATTGTTCTCACCGAAAGTGCCTGCGTATATTCATCCAAGGAAGTATCTCGTGGAAACACCA
CCGGTAGACGAGACTCCGGAGCCATCGGCAGAGAACCAATCCACAGAGGGGACACCTGAACAA
CCACCACTTATAACCGAGGATGAGACCAGGACTAGAACGCCTGAGCCGATCATCATCGAAGAGG
AAGAAGAGGATAGCATAAGTTTGCTGTCAGATGGCCCGACCCACCAGGTGCTGCAAGTCGAGGC
AGACATTCACGGGCCGCCCTCTGTATCTAGCTCATCCTGGTCCATTCCTCATGCATCCGACTTTG
ATGTGGACAGTTTATCCATACTTGACACCCTGGAGGGAGCTAGCGTGACCAGCGGGGCAACGTC
AGCCGAGACTAACTCTTACTTCGCAAAGAGTATGGAGTTTCTGGCGCGACCGGTGCCTGCGCCT
CGAACAGTATTCAGGAACCCTCCACATCCCGCTCCGCGCACAAGAACACCGTCACTTGCACCCA
GCAGGGCCTGCTCGAGAACCAGCCTAGTTTCCACCCCGCCAGGCGTGAATAGGGTGATCACTA
GAGAGGAGCTCGAGGCGCTTACCCCGTCACGCACTCCTAGCAGGTCGGTCTCGAGAACCAGCC
TGGTCTCCAACCCGCCAGGCGTAAATAGGGTGATTACAAGAGAGGAGTTTGAGGCGTTCGTAGC
ACAACAACAATGACGGTTTGATGCGGGTGCATACATCTTTTCCTCCGACACCGGTCAAGGGCATT
TACAACAAAAATCAGTAAGGCAAACGGTGCTATCCGAAGTGGTGTTGGAGAGGACCGAATTGGA
GATTTCGTATGCCCCGCGCCTCGACCAAGAAAAAGAAGAATTACTACGCAAGAAATTACAGTTAA
ATCCCACACCTGCTAACAGAAGCAGATACCAGTCCAGGAAGGTGGAGAACATGAAAGCCATAAC
AGCTAGACGTATTCTGCAAGGCCTAGGGCATTATTTGAAGGCAGAAGGAAAAGTGGAGTGCTAC
CGAACCCTGCATCCTGTTCCTTTGTATTCATCTAGTGTGAACCGTGCCTTTTCAAGCCCCAAGGT
CGCAGTGGAAGCCTGTAACGCCATGTTGAAAGAGAACTTTCCGACTGTGGCTTCTTACTGTATTA
TTCCAGAGTACGATGCCTATTTGGACATGGTTGACGGAGCTTCATGCTGCTTAGACACTGCCAGT
TTTTGCCCTGCAAAGCTGCGCAGCTTTCCAAAGAAACACTCCTATTTGGAACCCACAATACGATC
GGCAGTGCCTTCAGCGATCCAGAACACGCTCCAGAACGTCCTGGCAGCTGCCACAAAAAGAAAT
TGCAATGTCACGCAAATGAGAGAATTGCCCGTATTGGATTCGGCGGCCTTTAATGTGGAATGCTT
CAAGAAATATGCGTGTAATAATGAATATTGGGAAACGTTTAAAGAAAACCCCATCAGGCTTACTGA
AGAAAACGTGGTAAATTACATTACCAAATTAAAAGGACCAAAAGCTGCTGCTCTTTTTGCGAAGAC
ACATAATTTGAATATGTTGCAGGACATACCAATGGACAGGTTTGTAATGGACTTAAAGAGAGACG
TGAAAGTGACTCCAGGAACAAAACATACTGAAGAACGGCCCAAGGTACAGGTGATCCAGGCTGC
CGATCCGCTAGCAACAGCGTATCTGTGCGGAATCCACCGAGAGCTGGTTAGGAGATTAAATGCG
GTCCTGCTTCCGAACATTCATACACTGTTTGATATGTCGGCTGAAGACTTTGACGCTATTATAGCC
GAGCACTTCCAGCCTGGGGATTGTGTTCTGGAAACTGACATCGCGTCGTTTGATAAAAGTGAGG
ACGACGCCATGGCTCTGACCGCGTTAATGATTCTGGAAGACTTAGGTGTGGACGCAGAGCTGTT
GACGCTGATTGAGGCGGCTTTCGGCGAAATTTCATCAATACATTTGCCCACTAAAACTAAATTTAA
ATTCGGAGCCATGATGAAATCTGGAATGTTCCTCACACTGTTTGTGAACACAGTCATTAACATTGT
AATCGCAAGCAGAGTGTTGAGAGAACGGCTAACCGGATCACCATGTGCAGCATTCATTGGAGAT
GACAATATCGTGAAAGGAGTCAAATCGGACAAATTAATGGCAGACAGGTGCGCCACCTGGTTGA
ATATGGAAGTCAAGATTATAGATGCTGTGGTGGGCGAGAAAGCGCCTTATTTCTGTGGAGGGTTT
ATTTTGTGTGACTCCGTGACCGGCACAGCGTGCCGTGTGGCAGACCCCCTAAAAAGGCTGTTTA
AGCTTGGCAAACCTCTGGCAGCAGACGATGAACATGATGATGACAGGAGAAGGGCATTGCATGA
AGAGTCAACACGCTGGAACCGAGTGGGTATTCTTTCAGAGCTGTGCAAGGCAGTAGAATCAAGG
TATGAAACCGTAGGAACTTCCATCATAGTTATGGCCATGACTACTCTAGCTAGCAGTGTTAAATCA
TTCAGCTACCTGAGAGGGGCCCCTATAACTCTCTACGGCTAACCTGAATGGACTACGACATAGTC
TAGTCCGCCAAGTCTGTTTAAACAGCATAT
GGCGCGCCTAAACGAACGCCACCATGGTTAATCTTACAACCAGAACTCAATTACCCCCTGCAT
ACACTAATTCTTTCACACGTGGTGTTTATTACCCTGACAAAGTTTTCAGATCCTCAGTTTTACAT
TCAACTCAGGACTTGTTCTTACCTTTCTTTTCCAATGTTACTTGGTTCCATGCTATACATGTCTCT
GGGACCAATGGTACTAAGAGGTTTGATAACCCTGTCCTACCATTTAATGATGGTGTTTATTTTG
CTTCCATTGAGAAGTCTAACATAATAAGAGGCTGGATTTTTGGTACTACTTTAGATTCGAAGAC
CCAGTCCCTACTTATTGTTAATAACGCTACTAATGTTGTTATTAAAGTCTGTGAATTTCAATTTTG
TAATGATCCATTTTTGGGTGTTTATTACCACAAAAACAACAAAAGTTGGATGAAAAGTGAGTTC
AGAGTTTATTCTAGTGCGAATAATTGCACTTTTGAATATGTCTCTCAGCCTTTTCTTATGGACCT
TGAAGGAAAACAGGGTAATTTCAAAAATCTTAGGGAATTTGTGTTTAAGAATATTGATGGTTAT
TTTAAAATATATTCTAAGCACACGCCTATTAATTTAGTGCGTGATCTCCCTCAGGGTTTTTCGGC
TTTAGAACCATTGGTAGATTTGCCAATAGGTATTAACATCACTAGGTTTCAAACTTTACTTGCTT
TACATAGAAGTTATTTGACTCCTGGTGATTCTTCTTCAGGTTGGACAGCTGGTGCTGCAGCTTA
TTATGTGGGTTATCTTCAACCTAGGACTTTTCTATTAAAATATAATGAAAATGGAACCATTACAG
ATGCTGTAGACTGTGCACTTGACCCTCTCTCAGAAACAAAGTGTACGTTGAAATCCTTCACTGT
AGAAAAAGGAATCTATCAAACTTCTAACTTTAGAGTCCAACCAACAGAATCTATTGTTAGATTT
CCTAATATTACAAACTTGTGCCCTTTTGGTGAAGTTTTTAACGCCACCAGATTTGCATCTGTTTA
TGCTTGGAACAGGAAGAGAATCAGCAACTGTGTTGCTGATTATTCTGTCCTATATAATTCCGCA
TCATTTTCCACTTTTAAGTGTTATGGAGTGTCTCCTACTAAATTAAATGATCTCTGCTTTACTAAT
GTCTATGCAGATTCATTTGTAATTAGAGGTGATGAAGTCAGACAAATCGCTCCAGGGCAAACT
GGAAAGATTGCTGATTATAATTATAAATTACCAGATGATTTTACAGGCTGCGTTATAGCTTGGA
ATTCTAACAAACTTGATTCTAAGGTTGGTGGTAATTATAATTACCGCTATAGATTGTTTAGGAAG
TCTAATCTCAAACCTTTTGAGAGAGATATTTCAACTGAAATCTATCAGGCCGGTAGCACACCTT
GTAATGGTGTTCAGGGTTTTAATTGTTACTTTCCTTTACAATCATATGGTTTCCAACCCACTTAT
GGTGTTGGTTACCAACCATACAGAGTAGTAGTACTTTCTTTTGAACTTCTACATGCACCAGCAA
CTGTTTGTGGACCTAAAAAGTCTACTAATTTGGTTAAAAACAAATGTGTCAATTTCAACTTCAAT
GGTTTAACAGGCACAGGTGTTCTTACTGAGTCTAACAAAAAGTTTCTGCCTTTCCAACAATTTG
GCAGAGACATTGATGACACTACTGATGCTGTCCGTGATCCACAGACACTTGAGATTCTTGACAT
TACACCATGTTCTTTTGGTGGTGTCAGTGTTATAACACCAGGAACAAATACTTCTAACCAGGTT
GCTGTTCTTTATCAGGGTGTTAACTGCACAGAAGTCCCTGTTGCTATTCATGCAGATCAACTTA
CTCCTACTTGGCGTGTTTATTCTACAGGTTCTAATGTTTTTCAAACACGTGCAGGCTGTTTAATA
GGGGCTGAACATGTCAACAACTCATATGAGTGTGACATACCCATTGGTGCAGGTATATGCGCT
AGTTATCAGACTCAGACTAATTCTAGTGTAGCTAGTCAATCCATCATTGCCTACACTATGTCACT
TGGTGCAGAAAATTCAGTTGCTTACTCTAATAACTCTATTGCCATACCCATTAATTTTACTATTA
GTGTTACCACAGAAATTCTACCAGTGTCTATGACCAAGACATCAGTAGATTGTACAATGTACAT
TTGTGGTGATTCAACTGAATGCAGCAATCTTTTGTTGCAATATGGCAGTTTTTGTACACAATTAA
ACCGTGCTTTAACTGGAATAGCTGTTGAACAAGACAAAAACACCCAAGAAGTTTTTGCACAAG
TCAAACAAATTTACAAAACACCACCAATTAAAGATTTTGGTGGTTTTAATTTTTCACAAATATTA
CCAGATCCATCAAAACCAAGCAAGAGGTCATTTATTGAAGATCTACTTTTCAACAAAGTGACAC
TTGCAGATGCTGGCTTCATCAAACAATATGGTGATTGCCTTGGTGATATTGCTGCTAGAGACCT
CATTTGTGCACAAAAGTTTAACGGCCTTACTGTTTTGCCACCTTTGCTCACAGATGAAATGATT
GCTCAATACACTTCTGCACTGTTAGCGGGTACAATCACTTCTGGTTGGACCTTTGGTGCAGGTG
CTGCATTACAAATACCATTTGCTATGCAAATGGCTTATAGGTTTAATGGTATTGGAGTTACACA
GAATGTTCTCTATGAGAACCAAAAATTGATTGCCAACCAATTTAATAGTGCTATTGGCAAAATT
CAAGACTCACTTTCTTCCACAGCAAGTGCACTTGGAAAACTTCAAGATGTGGTCAACCAAAAT
GCACAAGCTTTAAACACGCTTGTTAAACAACTTAGCTCCAATTTTGGTGCAATTTCAAGTGTTTT
AAATGATATCCTTGCACGTCTTGACAAAGTTGAGGCTGAAGTGCAAATTGATAGGTTGATCACA
GGCAGACTTCAAAGTTTGCAGACATATGTGACTCAACAATTAATTAGAGCTGCAGAAATCAGA
GCTTCTGCTAATCTTGCTGCTACTAAAATGTCAGAGTGTGTACTTGGACAATCAAAAAGAGTTG
ATTTTTGTGGAAAGGGCTATCATCTTATGTCCTTCCCTCAGTCAGCACCTCATGGTGTAGTCTTC
TTGCATGTGACTTATGTCCCTGCACACGAAAAGAACTTCACAACTGCTCCTGCCATTTGTCATG
ATGGAAAAGCACACTTTCCTCGTGAAGGTGTCTTTGTTTCAAATGGCACACACTGGTTTGTAAC
ACAAAGGAATTTTTATGAACCACAAATCATTACTACACACAACACATTTGTGTCTGGTAACTGT
GATGTTGTAATAGGAATTGTCAACAACACAGTTTATGATCCTTTGCAACCTGAATTAGACTCATT
CAAGGAGGAGTTAGATAAATATTTTAAGAATCATACATCACCAGATGTTGATTTAGGTGACATC
TCTGGCATTAATGCTTCAGTTGTAAACATTCAAAAAGAAATTGACCGCCTCAATGAGGTTGCCA
AGAATTTAAATGAATCTCTCATCGATCTCCAAGAACTTGGAAAGTATGAGCAGTATATAAAATG
GCCATCACGACTGGAGGAAGAACTGCGCCGACGCCTGACTGAATAATCTAGA
GTGTTTAAACCGACCCGGGCGGCCGCAACTAACTTAAGCTAGCAACGGTTTCCCTCTAGCGGGA
TCAATTCCGCCCCCCCCCCCTAACGTTACTGGCCGAAGCCGCTTGGAATAAGGCCGGTGTGCGT
TTGTCTATATGTTATTTTCCACCATATTGCCGTCTTTTGGCAATGTGAGGGCCCGGAAACCTGGC
CCTGTCTTCTTGACGAGCATTCCTAGGGGTCTTTCCCCTCTCGCCAAAGGAATGCAAGGTCTGTT
GAATGTCGTGAAGGAAGCAGTTCCTCTGGAAGCTTCTTGAAGACAAACAACGTCTGTAGCGACC
CTTTGCAGGCAGCGGAACCCCCCACCTGGCGACAGGTGCCTCTGCGGCCAAAAGCCACGTGTA
TAAGATACACCTGCAAAGGCGGCACAACCCCAGTGCCACGTTGTGAGTTGGATAGTTGTGGAAA
GAGTCAAATGGCTCTCCTCAAGCGTATTCAACAAGGGGCTGAAGGATGCCCAGAAGGTACCCCA
TTGTATGGGATCTGATCTGGGGCCTCGGTGCACATGCTTTACATGTGTTTAGTCGAGGTTAAAAA
AACGTCTAGGCCCCCCGAACCACGGGGACGTGGTTTTCCTTTGAAAAACACGATAATACCATGA
CCGAGTACAAGCCCACGGTGCGCCTCGCCACCCGCGACGACGTCCCCAGGGCCGTACGCACC
CTCGCCGCCGCGTTCGCCGACTACCCCGCCACGCGCCACACCGTCGATCCGGACCGCCACATC
GAGCGGGTCACCGAGCTGCAAGAACTCTTCCTCACGCGCGTCGGGCTCGACATCGGCAAGGTG
TGGGTCGCGGACGACGGCGCCGCGGTGGCGGTCTGGACCACGCCGGAGAGCGTCGAAGCGG
GGGCGGTGTTCGCCGAGATCGGCCCGCGCATGGCCGAGTTGAGCGGTTCCCGGCTGGCCGCG
CAGCAACAGATGGAAGGCCTCCTGGCGCCGCACCGGCCCAAGGAGCCCGCGTGGTTCCTGGC
CACCGTCGGCGTCTCGCCCGACCACCAGGGCAAGGGTCTGGGCAGCGCCGTCGTGCTCCCCG
GAGTGGAGGCGGCCGAGCGCGCCGGGGTGCCCGCCTTCCTGGAGACCTCCGCGCCCCGCAAC
CTCCCCTTCTACGAGCGGCTCGGCTTCACCGTCACCGCCGACGTCGAGGTGCCCGAAGGACCG
CGCACCTGGTGCATGACCCGCAAGCCCGGTGCCTGAGAATTGGCAAGCTGCTTACATAGAACTC
GCGGCGATTGGCATGCCGCCTTAAAATTTTTATTTTATTTTTTCTTTTCTTTTCCGAATCGGATTTT
GTTTTTAATATTTCAAAAAAAAAAAAAAAAAAAAAAAAAACGCGTCGAGGGGAATTAATTCTTGAA
GACGAAAGGGCCAGGTGGCACTTTTCGGGGAAATGTGCGCGGAACCCCTATTTGTTTATTTTTCT
AAATACATTCAAATATGTATCCGCTCATGAGACAATAACCCTGATAAATGCTTCAATAATATTGAAA
AAGGAAGAGTATGAGTATTCAACATTTCCGTGTCGCCCTTATTCCCTTTTTTGCGGCATTTTGCCT
TCCTGTTTTTGCTCACCCAGAAACGCTGGTGAAAGTAAAAGATGCTGAAGATCAGTTGGGTGCAC
GAGTGGGTTACATCGAACTGGATCTCAACAGCGGTAAGATCCTTGAGAGTTTTCGCCCCGAAGA
ACGTTTTCCAATGATGAGCACTTTTAAAGTTCTGCTATGTGGCGCGGTATTATCCCGTGTTGACG
CCGGGCAAGAGCAACTCGGTCGCCGCATACACTATTCTCAGAATGACTTGGTTGAGTACTCACC
AGTCACAGAAAAGCATCTTACGGATGGCATGACAGTAAGAGAATTATGCAGTGCTGCCATAACCA
TGAGTGATAACACTGCGGCCAACTTACTTCTGACAACGATCGGAGGACCGAAGGAGCTAACCGC
TTTTTTGCACAACATGGGGGATCATGTAACTCGCCTTGATCGTTGGGAACCGGAGCTGAATGAAG
CCATACCAAACGACGAGCGTGACACCACGATGCCTGTAGCAATGGCAACAACGTTGCGCAAACT
ATTAACTGGCGAACTACTTACTCTAGCTTCCCGGCAACAATTAATAGACTGGATGGAGGCGGATA
AAGTTGCAGGACCACTTCTGCGCTCGGCCCTTCCGGCTGGCTGGTTTATTGCTGATAAATCTGG
AGCCGGTGAGCGTGGGTCTCGCGGTATCATTGCAGCACTGGGGCCAGATGGTAAGCCCTCCCG
TATCGTAGTTATCTACACGACGGGGAGTCAGGCAACTATGGATGAACGAAATAGACAGATCGCT
GAGATAGGTGCCTCACTGATTAAGCATTGGTAACTGTCAGACCAAGTTTACTCATATATACTTTAG
ATTGATTTAAAACTTCATTTTTAATTTAAAAGGATCTAGGTGAAGATCCTTTTTGATAATCTCATGA
CCAAAATCCCTTAACGTGAGTTTTCGTTCCACTGAGCGTCAGACCCCGTAGAAAAGATCAAAGGA
TCTTCTTGAGATCCTTTTTTTCTGCGCGTAATCTGCTGCTTGCAAACAAAAAAACCACCGCTACCA
GCGGTGGTTTGTTTGCCGGATCAAGAGCTACCAACTCTTTTTCCGAAGGTAACTGGCTTCAGCAG
AGCGCAGATACCAAATACTGTCCTTCTAGTGTAGCCGTAGTTAGGCCACCACTTCAAGAACTCTG
TAGCACCGCCTACATACCTCGCTCTGCTAATCCTGTTACCAGTGGCTGCTGCCAGTGGCGATAA
GTCGTGTCTTACCGGGTTGGACTCAAGACGATAGTTACCGGATAAGGCGCAGCGGTCGGGCTG
AACGGGGGGTTCGTGCACACAGCCCAGCTTGGAGCGAACGACCTACACCGAACTGAGATACCT
ACAGCGTGAGCTATGAGAAAGCGCCACGCTTCCCGAAGGGAGAAAGGCGGACAGGTATCCGGT
AAGCGGCAGGGTCGGAACAGGAGAGCGCACGAGGGAGCTTCCAGGGGGAAACGCCTGGTATC
TTTATAGTCCTGTCGGGTTTCGCCACCTCTGACTTGAGCGTCGATTTTTGTGATGCTCGTCAGGG
GGGCGGAGCCTATGGAAAAACGCCAGCAACGCGAGCTCGCGATCGCTTAATTAACGTTACATAA
CTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGG
TAAACTGCCCACTTGGCAGTACATCAAGTGTATCATATGCCAAGTACGCCCCCTATTGACGTCAA
TGACGGTAAATGGCCCGCCTGGCATTATGCCCAGTACATGACCTTATGGGACTTTCCTACTTGGC
AGTACATCTACGTATTAGTCATCGCTATTACCATGGTGATGCGGTTTTGGCAGTACATCAATGGG
CGTGGATAGCGGTTTGACTCACGGGGATTTCCAAGTCTCCACCCCATTGACGTCAATGGGAGTT
TGTTTTGGCACCAAAATCAACGGGACTTTCCAAAATGTCGTAACAACTCCGCCCCATTGACGCAA
ATGGGCGGTAGGCGTGTACGGTGGGAGGTCTATATAAGCAGAGCTTAATACGACTCACTATAGG
GCCGGCCATGGGCGGCGCATGAGAGAAGCCCAGACCAATTACCTACCCAAAATGGAGAAAGTT
CACGTTGACATCGAGGAAGACAGCCCATTCCTCAGAGCTTTGCAGCGGAGCTTCCCGCAGTTTG
AGGTAGAAGCCAAGCAGGTCACTGATAATGACCATGCTAATGCCAGAGCGTTTTCGCATCTGGC
TTCAAAACTGATCGAAACGGAGGTGGACCCATCCGACACGATCCTTGACATTGGAAGTGCGCCC
GCCCGCAGAATGTATTCTAAGCACAAGTATCATTGTATCTGTCCGATGAGATGTGCGGAAGATCC
GGACAGATTGTATAAGTATGCAACTAAGCTGAAGAAAAACTGTAAGGAAATAACTGATAAGGAAT
TGGACAAGAAAATGAAGGAGCTGGCCGCCGTCATGAGCGACCCTGACCTGGAAACTGAGACTAT
GTGCCTCCACGACGACGAGTCGTGTCGCTACGAAGGGCAAGTCGCTGTTTACCAGGATGTATAC
GCGGTTGACGGACCGACAAGTCTCTATCACCAAGCCAATAAGGGAGTTAGAGTCGCCTACTGGA
TAGGCTTTGACACCACCCCTTTTATGTTTAAGAACTTGGCTGGAGCATATCCATCATACTCTACCA
ACTGGGCCGACGAAACCGTGTT
Sequence encoding B.1.617.2 (Delta) spike protein variant of the SARS-COV-2 virus from
ProMab Biotechnologies, Inc. (SEQ ID NO: 13)
ATGTTTGTGTTCTTGGTGTTGCTTCCACTGGTCAGTTCCCAATGCGTTAATCTCAGAACCCGAACT
CAACTCCCACCCGCATATACAAATTCCTTCACCAGAGGAGTGTACTATCCTGACAAAGTGTTTCG
GTCAAGTGTCCTCCACTCTACTCAGGACCTCTTTCTGCCTTTCTTTTCTAACGTTACATGGTTTCA
TGCAATCCATGTGTCTGGGACAAACGGCACCAAACGCTTCGACAACCCTGTATTGCCATTCAATG
ATGGGGTGTACTTTGCCTCCATTGAGAAATCCAACATCATTCGAGGATGGATTTTCGGGACTACT
CTGGACTCAAAGACACAGAGCCTGCTGATCGTTAACAACGCCACAAACGTTGTCATCAAAGTGTG
CGAATTCCAGTTTTGCAATGATCCCTTCCTGGATGTGTACTATCACAAGAATAACAAGTCCTGGAT
GGAGAGCGGAGTCTACAGCAGCGCAAACAACTGCACCTTCGAGTACGTGAGTCAACCCTTTCTG
ATGGACCTGGAAGGGAAACAGGGAAACTTCAAGAACCTGAGAGAGTTTGTCTTTAAGAACATCG
ACGGCTATTTTAAGATCTATAGTAAGCATACGCCTATCAACCTGGTAAGGGATOTTCCCCAGGGC
TTTTCAGCCCTGGAACCTTTGGTTGACTTGCCTATTGGTATCAATATCACCAGATTTCAGACCCTT
CTGGCATTGCATCGGTCTTATCTTACTCCAGGTGATTCCTCCTCCGGGTGGACTGCCGGCGCCG
CTGCCTACTATGTCGGCTATCTGCAACCAAGAACGTTCCTGCTCAAGTACAACGAAAACGGCACT
ATTACGGATGCTGTTGATTGTGCCCTGGACCCTCTGTCTGAGACTAAATGCACCCTCAAGAGCTT
TACCGTTGAGAAGGGGATTTACCAAACCAGTAATTTCCGGGTCCAACCCACCGAAAGCATTGTG
CGGTTCCCAAATATCACCAATCTGTGTCCCTTTGGCGAAGTGTTCAATGCTACAAGGTTTGCTTC
TGTGTACGCATGGAATAGGAAACGCATCTCCAATTGTGTCGCTGATTACTCCGTGCTGTACAATT
CCGCCTCTTTCTCAACCTTCAAGTGTTATGGCGTTTCACCTACCAAACTTAACGACCTGTGCTTCA
CTAATGTGTATGCCGACTCTTTTGTGATACGAGGCGATGAAGTGAGACAGATTGCACCAGGGCA
GACCGGCAAAATTGCCGACTACAACTACAAGCTTCCAGATGACTTTACCGGATGTGTTATTGCAT
GGAACTCAAACAATCTGGATTCCAAGGTGGGTGGCAACTATAACTACCGCTATAGACTGTTCAGG
AAATCCAACCTGAAACCATTCGAGCGAGATATAAGCACAGAAATCTACCAGGCTGGAAGTAAACC
CTGCAACGGCGTGGAAGGGTTCAACTGCTACTTCCCATTGCAGAGTTACGGATTCCAGCCTACA
AACGGGGTGGGTTACCAACCCTATCGTGTCGTAGTCCTGAGTTTTGAGCTCCTCCATGCCCCAG
CCACAGTCTGTGGCCCCAAGAAAAGCACCAATCTGGTGAAGAACAAATGCGTGAACTTTAACTTT
AACGGACTCACAGGAACCGGCGTATTGACGGAGAGTAACAAGAAGTTCCTGCCATTCCAGCAGT
TCGGTCGCGATATTGCCGACACTACCGACGCTGTCCGAGATCCCCAGACATTGGAGATTCTTGA
TATCACACCCTGTAGTTTCGGCGGAGTGAGCGTGATTACGCCCGGAACCAATACCAGCAATCAG
GTTGCCGTCCTGTATCAGGGCGTGAATTGCACCGAGGTACCTGTCGCCATCCACGCTGACCAAC
TTACACCCACATGGCGAGTATATTCCACCGGCTCCAACGTCTTTCAGACACGTGCTGGATGTCTG
ATCGGTGCAGAACACGTTAATAATAGCTACGAGTGTGATATCCCCATCGGTGCTGGAATATGCGC
CTCTTATCAAACTCAAACCAACTCTCGTAGGCGGGCACGTAGTGTAGCATCCCAAAGTATCATTG
CCTACACAATGAGCCTCGGTGCTGAGAATTCTGTCGCCTACAGCAACAACTCCATTGCTATCCCT
ACTAACTTCACAATCAGTGTGACAACTGAAATTCTGCCCGTATCTATGACCAAAACAAGCGTTGA
CTGCACCATGTACATCTGTGGCGATTCTACCGAATGTAGCAATCTCCTCCTGCAATACGGATCAT
TCTGCACTCAGCTGAATCGTGCCCTCACAGGTATTGCAGTTGAGCAGGACAAGAATACGCAGGA
AGTGTTTGCCCAGGTGAAGCAAATCTACAAAACTCCACCCATAAAAGACTTTGGCGGATTCAATT
TCTCACAGATCCTGCCCGATCCCTCAAAACCCTCCAAGCGTAGCTTTATCGAGGATCTGCTCTTC
AACAAGGTAACCCTCGCAGATGCCGGTTTCATCAAGCAGTATGGCGATTGTCTGGGAGACATCG
CCGCTCGGGACCTGATCTGTGCACAGAAGTTCAATGGACTGACCGTGCTGCCTCCCTTGCTGAC
CGACGAGATGATAGCCCAATACACTAGCGCCCTGCTGGCCGGCACCATCACTTCTGGGTGGACA
TTCGGAGCTGGCGCTGCCCTTCAGATTCCTTTTGCTATGCAGATGGCCTACCGCTTTAACGGCAT
CGGTGTGACACAAAACGTTCTGTATGAAAACCAGAAACTCATCGCCAACCAGTTCAACAGTGCTA
TCGGTAAGATACAGGATAGCCTGTCATCCACTGCCAGCGCATTGGGAAAGTTGCAGAATGTAGT
GAACCAGAATGCCCAGGCACTTAACACCCTGGTGAAACAGCTCTCTTCAAATTTTGGTGCCATTT
CTAGCGTGCTGAATGACATACTGAGCCGGTTGGACAAGGTGGAGGCTGAAGTGCAGATTGATAG
GCTGATAACTGGGCGCCTTCAGTCTCTTCAGACCTATGTGACCCAGCAGCTCATCCGCGCTGCT
GAAATTCGCGCATCCGCTAACCTGGCAGCAACCAAAATGTCCGAGTGTGTGCTGGGTCAGTCTA
AGAGAGTGGACTTTTGCGGGAAGGGGTATCACCTGATGTCTTTTCCTCAGTCTGCACCCCATGG
TGTGGTCTTTCTGCACGTGACTTATGTCCCAGCTCAGGAAAAGAACTTCACTACAGCCCCAGCCA
TCTGCCACGATGGGAAAGCCCACTTTCCCAGGGAAGGCGTATTCGTGTCCAATGGTACTCATTG
GTTCGTCACTCAGAGAAATTTCTACGAGCCCCAGATTATAACCACTGACAATACATTTGTATCCG
GCAATTGTGATGTGGTTATCGGGATTGTGAATAATACTGTTTACGATCCTTTGCAGCCAGAGCTG
GACTCCTTCAAGGAGGAGCTTGACAAATATTTTAAGAATCACACATCACCTGACGTCGACCTCGG
AGATATTTCAGGAATCAATGCTTCCGTGGTCAATATTCAGAAGGAGATAGACAGGCTGAATGAGG
TTGCCAAGAACCTCAACGAGTCTCTGATCGATCTGCAGGAGTTGGGCAAGTACGAACAGTATATC
AAATGGCCATGGTACATTTGGCTTGGGTTCATTGCTGGGCTGATAGCTATCGTCATGGTGACAAT
TATGTTGTGTTGCATGACATCCTGCTGTAGTTGTCTGAAGGGCTGCTGCTCATGCGGCAGCTGTT
GCTAATGATAG
Sequence encoding eGFP from ProMab Biotechnologies, Inc.
ATGGTGAGCAAGGGCGAGGAGCTGTTCACCGGGGTGGTGCCCATCCTGGTCGAGCTGGACGG
CGACGTAAACGGCCACAAGTTCAGCGTGTCCGGCGAGGGCGAGGGCGATGCCACCTACGGCAA
GCTGACCCTGAAGTTCATCTGCACCACCGGCAAGCTGCCCGTGCCCTGGCCCACCCTCGTGAC
CACCCTGACCTACGGCGTGCAGTGCTTCAGCCGCTACCCCGACCACATGAAGCAGCACGACTTC
TTCAAGTCCGCCATGCCCGAAGGCTACGTCCAGGAGCGCACCATCTTCTTCAAGGACGACGGCA
ACTACAAGACCCGCGCCGAGGTGAAGTTCGAGGGCGACACCCTGGTGAACCGCATCGAGCTGA
AGGGCATCGACTTCAAGGAGGACGGCAACATCCTGGGGCACAAGCTGGAGTACAACTACAACA
GCCACAACGTCTATATCATGGCCGACAAGCAGAAGAACGGCATCAAGGTGAACTTCAAGATCCG
CCACAACATCGAGGACGGCAGCGTGCAGCTCGCCGACCACTACCAGCAGAACACCCCCATCGG
CGACGGCCCCGTGCTGCTGCCCGACAACCACTACCTGAGCACCCAGTCCGCCCTGAGCAAAGA
CCCCAACGAGAAGCGCGATCACATGGTCCTGCTGGAGTTCGTGACCGCCGCCGGGATCACTCT
CGGCATGGACGAGCTGTACAAGTAA
Sequence encoding B.1.1.529 (Omicron) Spike variant of SARS COV-2 Spike vaccine (SEQ ID NO: 14)
AACGGCTCGTAACATAGGCCTATGCAGCTCTGACGTTATGGAGCGGTCACGTAGAGGGATGTCC
ATTCTTAGAAAGAAGTATTTGAAACCATCCAACAATGTTCTATTCTCTGTTGGCTCGACCATCTAC
CACGAGAAGAGGGACTTACTGAGGAGCTGGCACCTGCCGTCTGTATTTCACTTACGTGGCAAGC
AAAATTACACATGTCGGTGTGAGACTATAGTTAGTTGCGACGGGTACGTCGTTAAAAGAATAGCT
ATCAGTCCAGGCCTGTATGGGAAGCCTTCAGGCTATGCTGCTACGATGCACCGCGAGGGATTCT
TGTGCTGCAAAGTGACAGACACATTGAACGGGGAGAGGGTCTCTTTTCCCGTGTGCACGTATGT
GCCAGCTACATTGTGTGACCAAATGACTGGCATACTGGCAACAGATGTCAGTGCGGACGACGCG
CAAAAACTGCTGGTTGGGCTCAACCAGCGTATAGTCGTCAACGGTCGCACCCAGAGAAACACCA
ATACCATGAAAAATTACCTTTTGCCCGTAGTGGCCCAGGCATTTGCTAGGTGGGCAAAGGAATAT
AAGGAAGATCAAGAAGATGAAAGGCCACTAGGACTACGAGATAGACAGTTAGTCATGGGGTGTT
GTTGGGCTTTTAGAAGGCACAAGATAACATCTATTTATAAGCGCCCGGATACCCAAACCATCATC
AAAGTGAACAGCGATTTCCACTCATTCGTGCTGCCCAGGATAGGCAGTAACACATTGGAGATCG
GGCTGAGAACAAGAATCAGGAAAATGTTAGAGGAGCACAAGGAGCCGTCACCTCTCATTACCGC
CGAGGACGTACAAGAAGCTAAGTGCGCAGCCGATGAGGCTAAGGAGGTGCGTGAAGCCGAGGA
GTTGCGCGCAGCTCTACCACCTTTGGCAGCTGATGTTGAGGAGCCCACTCTGGAGGCAGACGT
CGACTTGATGTTACAAGAGGCTGGGGCCGGCTCAGTGGAGACACCTCGTGGCTTGATAAAGGTT
ACCAGCTACGATGGCGAGGACAAGATCGGCTCTTACGCTGTGCTTTCTCCGCAGGCTGTACTCA
AGAGTGAAAAATTATCTTGCATCCACCCTCTCGCTGAACAAGTCATAGTGATAACACACTCTGGC
CGAAAAGGGCGTTATGCCGTGGAACCATACCATGGTAAAGTAGTGGTGCCAGAGGGACATGCAA
TACCCGTCCAGGACTTTCAAGCTCTGAGTGAAAGTGCCACCATTGTGTACAACGAACGTGAGTTC
GTAAACAGGTACCTGCACCATATTGCCACACATGGAGGAGCGCTGAACACTGATGAAGAATATTA
CAAAACTGTCAAGCCCAGCGAGCACGACGGCGAATACCTGTACGACATCGACAGGAAACAGTGC
GTCAAGAAAGAACTAGTCACTGGGCTAGGGCTCACAGGCGAGCTGGTGGATCCTCCCTTCCATG
AATTCGCCTACGAGAGTCTGAGAACACGACCAGCCGCTCCTTACCAAGTACCAACCATAGGGGT
GTATGGCGTGCCAGGATCAGGCAAGTCTGGCATCATTAAAAGCGCAGTCACCAAAAAAGATCTA
GTGGTGAGCGCCAAGAAAGAAAACTGTGCAGAAATTATAAGGGACGTCAAGAAAATGAAAGGGC
TGGACGTCAATGCCAGAACTGTGGACTCAGTGCTCTTGAATGGATGCAAACACCCCGTAGAGAC
CCTGTATATTGACGAAGCTTTTGCTTGTCATGCAGGTACTCTCAGAGCGCTCATAGCCATTATAA
GACCTAAAAAGGCAGTGCTCTGCGGGGATCCCAAACAGTGCGGTTTTTTTAACATGATGTGCCT
GAAAGTGCATTTTAACCACGAGATTTGCACACAAGTCTTCCACAAAAGCATCTCTCGCCGTTGCA
CTAAATCTGTGACTTCGGTCGTCTCAACCTTGTTTTACGACAAAAAAATGAGAACGACGAATCCG
AAAGAGACTAAGATTGTGATTGACACTACCGGCAGTACCAAACCTAAGCAGGACGATCTCATTCT
CACTTGTTTCAGAGGGTGGGTGAAGCAGTTGCAAATAGATTACAAAGGCAACGAAATAATGACG
GCAGCTGCCTCTCAAGGGCTGACCCGTAAAGGTGTGTATGCCGTTCGGTACAAGGTGAATGAAA
ATCCTCTGTACGCACCCACCTCAGAACATGTGAACGTCCTACTGACCCGCACGGAGGACCGCAT
CGTGTGGAAAACACTAGCCGGCGACCCATGGATAAAAACACTGACTGCCAAGTACCCTGGGAAT
TTCACTGCCACGATAGAGGAGTGGCAAGCAGAGCATGATGCCATCATGAGGCACATCTTGGAGA
GACCGGACCCTACCGACGTCTTCCAGAATAAGGCAAACGTGTGTTGGGCCAAGGCTTTAGTGCC
GGTGCTGAAGACCGCTGGCATAGACATGACCACTGAACAATGGAACACTGTGGATTATTTTGAAA
CGGACAAAGCTCACTCAGCAGAGATAGTATTGAACCAACTATGCGTGAGGTTCTTTGGACTCGAT
CTGGACTCCGGTCTATTTTCTGCACCCACTGTTCCGTTATCCATTAGGAATAATCACTGGGATAA
CTCCCCGTCGCCTAACATGTACGGGCTGAATAAAGAAGTGGTCCGTCAGCTCTCTCGCAGGTAC
CCACAACTGCCTCGGGCAGTTGCCACTGGAAGAGTCTATGACATGAACACTGGTACACTGCGCA
ATTATGATCCGCGCATAAACCTAGTACCTGTAAACAGAAGACTGCCTCATGCTTTAGTCCTCCAC
CATAATGAACACCCACAGAGTGACTTTTCTTCATTCGTCAGCAAATTGAAGGGCAGAACTGTCCT
GGTGGTCGGGGAAAAGTTGTCCGTCCCAGGCAAAATGGTTGACTGGTTGTCAGACCGGCCTGA
GGCTACCTTCAGAGCTCGGCTGGATTTAGGCATCCCAGGTGATGTGCCCAAATATGACATAATAT
TTGTTAATGTGAGGACCCCATATAAATACCATCACTATCAGCAGTGTGAAGACCATGCCATTAAG
CTTAGCATGTTGACCAAGAAAGCTTGTCTGCATCTGAATCCCGGCGGAACCTGTGTCAGCATAG
GTTATGGTTACGCTGACAGGGCCAGCGAAAGCATCATTGGTGCTATAGCGCGGCAGTTCAAGTT
TTCCCGGGTATGCAAACCGAAATCCTCACTTGAAGAGACGGAAGTTCTGTTTGTATTCATTGGGT
ACGATCGCAAGGCCCGTACGCACAATTCTTACAAGCTTTCATCAACCTTGACCAACATTTATACA
GGTTCCAGACTCCACGAAGCCGGATGTGCACCCTCATATCATGTGGTGCGAGGGGATATTGCCA
CGGCCACCGAAGGAGTGATTATAAATGCTGCTAACAGCAAAGGACAACCTGGCGGAGGGGTGT
GCGGAGCGCTGTATAAGAAATTCCCGGAAAGCTTCGATTTACAGCCGATCGAAGTAGGAAAAGC
GCGACTGGTCAAAGGTGCAGCTAAACATATCATTCATGCCGTAGGACCAAACTTCAACAAAGTTT
CGGAGGTTGAAGGTGACAAACAGTTGGCAGAGGCTTATGAGTCCATCGCTAAGATTGTCAACGA
TAACAATTACAAGTCAGTAGCGATTCCACTGTTGTCCACCGGCATCTTTTCCGGGAACAAAGATC
GACTAACCCAATCATTGAACCATTTGCTGACAGCTTTAGACACCACTGATGCAGATGTAGCCATA
TACTGCAGGGACAAGAAATGGGAAATGACTCTCAAGGAAGCAGTGGCTAGGAGAGAAGCAGTG
GAGGAGATATGCATATCCGACGACTCTTCAGTGACAGAACCTGATGCAGAGCTGGTGAGGGTGC
ATCCGAAGAGTTCTTTGGCTGGAAGGAAGGGCTACAGCACAAGCGATGGCAAAACTTTCTCATAT
TTGGAAGGGACCAAGTTTCACCAGGCGGCCAAGGATATAGCAGAAATTAATGCCATGTGGCCCG
TTGCAACGGAGGCCAATGAGCAGGTATGCATGTATATCCTCGGAGAAAGCATGAGCAGTATTAG
GTCGAAATGCCCCGTCGAAGAGTCGGAAGCCTCCACACCACCTAGCACGCTGCCTTGCTTGTGC
ATCCATGCCATGACTCCAGAAAGAGTACAGCGCCTAAAAGCCTCACGTCCAGAACAAATTACTGT
GTGCTCATCCTTTCCATTGCCGAAGTATAGAATCACTGGTGTGCAGAAGATCCAATGCTCCCAGC
CTATATTGTTCTCACCGAAAGTGCCTGCGTATATTCATCCAAGGAAGTATCTCGTGGAAACACCA
CCGGTAGACGAGACTCCGGAGCCATCGGCAGAGAACCAATCCACAGAGGGGACACCTGAACAA
CCACCACTTATAACCGAGGATGAGACCAGGACTAGAACGCCTGAGCCGATCATCATCGAAGAGG
AAGAAGAGGATAGCATAAGTTTGCTGTCAGATGGCCCGACCCACCAGGTGCTGCAAGTCGAGGC
AGACATTCACGGGCCGCCCTCTGTATCTAGCTCATCCTGGTCCATTCCTCATGCATCCGACTTTG
ATGTGGACAGTTTATCCATACTTGACACCCTGGAGGGAGCTAGCGTGACCAGCGGGGCAACGTC
AGCCGAGACTAACTCTTACTTCGCAAAGAGTATGGAGTTTCTGGCGCGACCGGTGCCTGCGCCT
CGAACAGTATTCAGGAACCCTCCACATCCCGCTCCGCGCACAAGAACACCGTCACTTGCACCCA
GCAGGGCCTGCTCGAGAACCAGCCTAGTTTCCACCCCGCCAGGCGTGAATAGGGTGATCACTA
GAGAGGAGCTCGAGGCGCTTACCCCGTCACGCACTCCTAGCAGGTCGGTCTCGAGAACCAGCC
TGGTCTCCAACCCGCCAGGCGTAAATAGGGTGATTACAAGAGAGGAGTTTGAGGCGTTCGTAGC
ACAACAACAATGACGGTTTGATGCGGGTGCATACATCTTTTCCTCCGACACCGGTCAAGGGCATT
TACAACAAAAATCAGTAAGGCAAACGGTGCTATCCGAAGTGGTGTTGGAGAGGACCGAATTGGA
GATTTCGTATGCCCCGCGCCTCGACCAAGAAAAAGAAGAATTACTACGCAAGAAATTACAGTTAA
ATCCCACACCTGCTAACAGAAGCAGATACCAGTCCAGGAAGGTGGAGAACATGAAAGCCATAAC
AGCTAGACGTATTCTGCAAGGCCTAGGGCATTATTTGAAGGCAGAAGGAAAAGTGGAGTGCTAC
CGAACCCTGCATCCTGTTCCTTTGTATTCATCTAGTGTGAACCGTGCCTTTTCAAGCCCCAAGGT
CGCAGTGGAAGCCTGTAACGCCATGTTGAAAGAGAACTTTCCGACTGTGGCTTCTTACTGTATTA
TTCCAGAGTACGATGCCTATTTGGACATGGTTGACGGAGCTTCATGCTGCTTAGACACTGCCAGT
TTTTGCCCTGCAAAGCTGCGCAGCTTTCCAAAGAAACACTCCTATTTGGAACCCACAATACGATC
GGCAGTGCCTTCAGCGATCCAGAACACGCTCCAGAACGTCCTGGCAGCTGCCACAAAAAGAAAT
TGCAATGTCACGCAAATGAGAGAATTGCCCGTATTGGATTCGGCGGCCTTTAATGTGGAATGCTT
CAAGAAATATGCGTGTAATAATGAATATTGGGAAACGTTTAAAGAAAACCCCATCAGGCTTACTGA
AGAAAACGTGGTAAATTACATTACCAAATTAAAAGGACCAAAAGCTGCTGCTCTTTTTGCGAAGAC
ACATAATTTGAATATGTTGCAGGACATACCAATGGACAGGTTTGTAATGGACTTAAAGAGAGACG
TGAAAGTGACTCCAGGAACAAAACATACTGAAGAACGGCCCAAGGTACAGGTGATCCAGGCTGC
CGATCCGCTAGCAACAGCGTATCTGTGCGGAATCCACCGAGAGCTGGTTAGGAGATTAAATGCG
GTCCTGCTTCCGAACATTCATACACTGTTTGATATGTCGGCTGAAGACTTTGACGCTATTATAGCC
GAGCACTTCCAGCCTGGGGATTGTGTTCTGGAAACTGACATCGCGTCGTTTGATAAAAGTGAGG
ACGACGCCATGGCTCTGACCGCGTTAATGATTCTGGAAGACTTAGGTGTGGACGCAGAGCTGTT
GACGCTGATTGAGGCGGCTTTCGGCGAAATTTCATCAATACATTTGCCCACTAAAACTAAATTTAA
ATTCGGAGCCATGATGAAATCTGGAATGTTCCTCACACTGTTTGTGAACACAGTCATTAACATTGT
AATCGCAAGCAGAGTGTTGAGAGAACGGCTAACCGGATCACCATGTGCAGCATTCATTGGAGAT
GACAATATCGTGAAAGGAGTCAAATCGGACAAATTAATGGCAGACAGGTGCGCCACCTGGTTGA
ATATGGAAGTCAAGATTATAGATGCTGTGGTGGGCGAGAAAGCGCCTTATTTCTGTGGAGGGTTT
ATTTTGTGTGACTCCGTGACCGGCACAGCGTGCCGTGTGGCAGACCCCCTAAAAAGGCTGTTTA
AGCTTGGCAAACCTCTGGCAGCAGACGATGAACATGATGATGACAGGAGAAGGGCATTGCATGA
AGAGTCAACACGCTGGAACCGAGTGGGTATTCTTTCAGAGCTGTGCAAGGCAGTAGAATCAAGG
TATGAAACCGTAGGAACTTCCATCATAGTTATGGCCATGACTACTCTAGCTAGCAGTGTTAAATCA
TTCAGCTACCTGAGAGGGGCCCCTATAACTCTCTACGGCTAACCTGAATGGACTACGACATAGTC
TAGTCCGCCAAGTCTGTTTAAACAGCATATGGGCGCGCCCTCAGCATCGATTCAATTCGCCACCA
TGTTTGTTTTTCTTGTTTTATTGCCACTAGTCTCTAGTCAGTGTGTTAATCTTACAACCAGAACTCA
ATTACCCCCTGCATACACTAATTCTTTCACACGTGGTGTTTATTACCCTGACAAAGTTTTCAGATC
CTCAGTTTTACATTCAACTCAGGACTTGTTCTTACCTTTCTTTTCCAATGTTACTTGGTTCCATGTT
ATCTCTGGGACCAATGGTACTAAGAGGTTTGATAACCCTGTCCTACCATTTAATGATGGTGTTTAT
TTTGCTTCCATTGAGAAGTCTAACATAATAAGAGGCTGGATTTTTGGTACTACTTTAGATTCGAAG
ACCCAGTCCCTACTTATTGTTAATAACGCTACTAATGTTGTTATTAAAGTCTGTGAATTTCAATTTT
GTAATGATCCATTTTTGGACCACAAAAACAACAAAAGTTGGATGGAAAGTGAGTTCAGAGTTTATT
CTAGTGCGAATAATTGCACTTTTGAATATGTCTCTCAGCCTTTTCTTATGGACCTTGAAGGAAAAC
AGGGTAATTTCAAAAATCTTAGGGAATTTGTGTTTAAGAATATTGATGGTTATTTTAAAATATATTC
TAAGCACACGCCTATTATAGTGCGTGAGCCAGAAGATCTCCCTCAGGGTTTTTCGGCTTTAGAAC
CATTGGTAGATTTGCCAATAGGTATTAACATCACTAGGTTTCAAACTTTACTTGCTTTACATAGAA
GTTATTTGACTCCTGGTGATTCTTCTTCAGGTTGGACAGCTGGTGCTGCAGCTTATTATGTGGGT
TATCTTCAACCTAGGACTTTTCTATTAAAATATAATGAAAATGGAACCATTACAGATGCTGTAGACT
GTGCACTTGACCCTCTCTCAGAAACAAAGTGTACGTTGAAATCCTTCACTGTAGAAAAAGGAATC
TATCAAACTTCTAACTTTAGAGTCCAACCAACAGAATCTATTGTTAGATTTCCTAATATTACAAACT
TGTGCCCTTTTGATGAAGTTTTTAACGCCACCAGATTTGCATCTGTTTATGCTTGGAACAGGAAGA
GAATCAGCAACTGTGTTGCTGATTATTCTGTCCTATATAATCTCGCACCATTTTTCACTTTTAAGTG
TTATGGAGTGTCTCCTACTAAATTAAATGATCTCTGCTTTACTAATGTCTATGCAGATTCATTTGTA
ATTAGAGGTGATGAAGTCAGACAAATCGCTCCAGGGCAAACTGGAAATATTGCTGATTATAATTA
TAAATTACCAGATGATTTTACAGGCTGCGTTATAGCTTGGAATTCTAACAAGCTTGATTCTAAGGT
TAGTGGTAATTATAATTACCTGTATAGATTGTTTAGGAAGTCTAATCTCAAACCTTTTGAGAGAGAT
ATTTCAACTGAAATCTATCAGGCCGGTAACAAACCTTGTAATGGTGTTGCAGGTTTTAATTGTTAC
TTTCCTTTACGATCATATAGTTTCCGACCCACTTATGGTGTTGGTCACCAACCATACAGAGTAGTA
GTACTTTCTTTTGAACTTCTACATGCACCAGCAACTGTTTGTGGACCTAAAAAGTCTACTAATTTG
GTTAAAAACAAATGTGTCAATTTCAACTTCAATGGTTTAAAAGGCACAGGTGTTCTTACTGAGTCT
AACAAAAAGTTTCTGCCTTTCCAACAATTTGGCAGAGACATTGCTGACACTACTGATGCTGTCCG
TGATCCACAGACACTTGAGATTCTTGACATTACACCATGTTCTTTTGGTGGTGTCAGTGTTATAAC
ACCAGGAACAAATACTTCTAACCAGGTTGCTGTTCTTTATCAGGGTGTTAACTGCACAGAAGTCC
CTGTTGCTATTCATGCAGATCAACTTACTCCTACTTGGCGTGTTTATTCTACAGGTTCTAATGTTTT
TCAAACACGTGCAGGCTGTTTAATAGGGGCTGAATATGTCAACAACTCATATGAGTGTGACATAC
CCATTGGTGCAGGTATATGCGCTAGTTATCAGACTCAGACTAAGTCTCATGCTAGTGTAGCTAGT
CAATCCATCATTGCCTACACTATGTCACTTGGTGCAGAAAATTCAGTTGCTTACTCTAATAACTCT
ATTGCCATACCCACAAATTTTACTATTAGTGTTACCACAGAAATTCTACCAGTGTCTATGACCAAG
ACATCAGTAGATTGTACAATGTACATTTGTGGTGATTCAACTGAATGCAGCAATCTTTTGTTGCAA
TATGGCAGTTTTTGTACACAATTAAAACGTGCTTTAACTGGAATAGCTGTTGAACAAGACAAAAAC
ACCCAAGAAGTTTTTGCACAAGTCAAACAAATTTACAAAACACCACCAATTAAATATTTTGGTGGT
TTTAATTTTTCACAAATATTACCAGATCCATCAAAACCAAGCAAGAGGTCATTTATTGAAGATCTAC
TTTTCAACAAAGTGACACTTGCAGATGCTGGCTTCATCAAACAATATGGTGATTGCCTTGGTGATA
TTGCTGCTAGAGACCTCATTTGTGCACAAAAGTTTAAAGGCCTTACTGTTTTGCCACCTTTGCTCA
CAGATGAAATGATTGCTCAATACACTTCTGCACTGTTAGCGGGTACAATCACTTCTGGTTGGACC
TTTGGTGCAGGTGCTGCATTACAAATACCATTTGCTATGCAAATGGCTTATAGGTTTAATGGTATT
GGAGTTACACAGAATGTTCTCTATGAGAACCAAAAATTGATTGCCAACCAATTTAATAGTGCTATT
GGCAAAATTCAAGACTCACTTTCTTCCACAGCAAGTGCACTTGGAAAACTTCAAGATGTGGTCAA
CCATAATGCACAAGCTTTAAACACGCTTGTTAAACAACTTAGCTCCAAATTTGGTGCAATTTCAAG
TGTTTTAAATGATATCTTTTCACGTCTTGACCCTCCTGAGGCTGAAGTGCAAATTGATAGGTTGAT
CACAGGCAGACTTCAAAGTTTGCAGACATATGTGACTCAACAATTAATTAGAGCTGCAGAAATCA
GAGCTTCTGCTAATCTTGCTGCTACTAAAATGTCAGAGTGTGTACTTGGACAATCAAAAAGAGTT
GATTTTTGTGGAAAGGGCTATCATCTTATGTCCTTCCCTCAGTCAGCACCTCATGGTGTAGTCTTC
TTGCATGTGACTTATGTCCCTGCACAAGAAAAGAACTTCACAACTGCTCCTGCCATTTGTCATGAT
GGAAAAGCACACTTTCCTCGTGAAGGTGTCTTTGTTTCAAATGGCACACACTGGTTTGTAACACA
AAGGAATTTTTATGAACCACAAATCATTACTACAGACAACACATTTGTGTCTGGTAACTGTGATGT
TGTAATAGGAATTGTCAACAACACAGTTTATGATCCTTTGCAACCTGAATTAGATTCATTCAAGGA
GGAGTTAGATAAATATTTTAAGAATCATACATCACCAGATGTTGATTTAGGTGACATCTCTGGCAT
TAATGCTTCAGTTGTAAACATTCAAAAAGAAATTGACCGCCTCAATGAGGTTGCCAAGAATTTAAA
TGAATCTCTCATCGATCTCCAAGAACTTGGAAAGTATGAGCAGTATATAAAATGGCCATGGTACAT
TTGGCTAGGTTTTATAGCTGGCTTGATTGCCATAGTAATGGTGACAATTATGCTTTGCTGTATGAC
CAGTTGCTGTAGTTGTCTCAAGGGCTGTTGTTCTTGTGGATCCTGCTGCAAATTTGATGAAGACG
ACTCTGAGCCAGTGCTCAAAGGAGTCAAATTACATTACACATCACGACTGGAGGAAGAACTGCG
CCGACGCCTGACTGAATAATCTAGAGTGTTTAAACCGACCCGGGCGGCCGCAACTAACTTAAGC
TAGCAACGGTTTCCCTCTAGCGGGATCAATTCCGCCCCCCCCCCCTAACGTTACTGGCCGAAGC
CGCTTGGAATAAGGCCGGTGTGCGTTTGTCTATATGTTATTTTCCACCATATTGCCGTCTTTTGGC
AATGTGAGGGCCCGGAAACCTGGCCCTGTCTTCTTGACGAGCATTCCTAGGGGTCTTTCCCCTC
TCGCCAAAGGAATGCAAGGTCTGTTGAATGTCGTGAAGGAAGCAGTTCCTCTGGAAGCTTCTTG
AAGACAAACAACGTCTGTAGCGACCCTTTGCAGGCAGCGGAACCCCCCACCTGGCGACAGGTG
CCTCTGCGGCCAAAAGCCACGTGTATAAGATACACCTGCAAAGGCGGCACAACCCCAGTGCCAC
GTTGTGAGTTGGATAGTTGTGGAAAGAGTCAAATGGCTCTCCTCAAGCGTATTCAACAAGGGGCT
GAAGGATGCCCAGAAGGTACCCCATTGTATGGGATCTGATCTGGGGCCTCGGTGCACATGCTTT
ACATGTGTTTAGTCGAGGTTAAAAAAACGTCTAGGCCCCCCGAACCACGGGGACGTGGTTTTCC
TTTGAAAAACACGATAATACCATGACCGAGTACAAGCCCACGGTGCGCCTCGCCACCCGCGACG
ACGTCCCCAGGGCCGTACGCACCCTCGCCGCCGCGTTCGCCGACTACCCCGCCACGCGCCAC
ACCGTCGATCCGGACCGCCACATCGAGCGGGTCACCGAGCTGCAAGAACTCTTCCTCACGCGC
GTCGGGCTCGACATCGGCAAGGTGTGGGTCGCGGACGACGGCGCCGCGGTGGCGGTCTGGAC
CACGCCGGAGAGCGTCGAAGCGGGGGCGGTGTTCGCCGAGATCGGCCCGCGCATGGCCGAGT
TGAGCGGTTCCCGGCTGGCCGCGCAGCAACAGATGGAAGGCCTCCTGGCGCCGCACCGGCCC
AAGGAGCCCGCGTGGTTCCTGGCCACCGTCGGCGTCTCGCCCGACCACCAGGGCAAGGGTCT
GGGCAGCGCCGTCGTGCTCCCCGGAGTGGAGGCGGCCGAGCGCGCCGGGGTGCCCGCCTTC
CTGGAGACCTCCGCGCCCCGCAACCTCCCCTTCTACGAGCGGCTCGGCTTCACCGTCACCGCC
GACGTCGAGGTGCCCGAAGGACCGCGCACCTGGTGCATGACCCGCAAGCCCGGTGCCTGAGA
ATTGGCAAGCTGCTTACATAGAACTCGCGGCGATTGGCATGCCGCCTTAAAATTTTTATTTTATTT
TTTCTTTTCTTTTCCGAATCGGATTTTGTTTTTAATATTTCAAAAAAAAAAAAAAAAAAAAAAAAAAC
GCGTCGAGGGGAATTAATTCTTGAAGACGAAAGGGCCAGGTGGCACTTTTCGGGGAAATGTGCG
CGGAACCCCTATTTGTTTATTTTTCTAAATACATTCAAATATGTATCCGCTCATGAGACAATAACCC
TGATAAATGCTTCAATAATATTGAAAAAGGAAGAGTATGAGTATTCAACATTTCCGTGTCGCCCTT
ATTCCCTTTTTTGCGGCATTTTGCCTTCCTGTTTTTGCTCACCCAGAAACGCTGGTGAAAGTAAAA
GATGCTGAAGATCAGTTGGGTGCACGAGTGGGTTACATCGAACTGGATCTCAACAGCGGTAAGA
TCCTTGAGAGTTTTCGCCCCGAAGAACGTTTTCCAATGATGAGCACTTTTAAAGTTCTGCTATGTG
GCGCGGTATTATCCCGTGTTGACGCCGGGCAAGAGCAACTCGGTCGCCGCATACACTATTCTCA
GAATGACTTGGTTGAGTACTCACCAGTCACAGAAAAGCATCTTACGGATGGCATGACAGTAAGAG
AATTATGCAGTGCTGCCATAACCATGAGTGATAACACTGCGGCCAACTTACTTCTGACAACGATC
GGAGGACCGAAGGAGCTAACCGCTTTTTTGCACAACATGGGGGATCATGTAACTCGCCTTGATC
GTTGGGAACCGGAGCTGAATGAAGCCATACCAAACGACGAGCGTGACACCACGATGCCTGTAG
CAATGGCAACAACGTTGCGCAAACTATTAACTGGCGAACTACTTACTCTAGCTTCCCGGCAACAA
TTAATAGACTGGATGGAGGCGGATAAAGTTGCAGGACCACTTCTGCGCTCGGCCCTTCCGGCTG
GCTGGTTTATTGCTGATAAATCTGGAGCCGGTGAGCGTGGGTCTCGCGGTATCATTGCAGCACT
GGGGCCAGATGGTAAGCCCTCCCGTATCGTAGTTATCTACACGACGGGGAGTCAGGCAACTATG
GATGAACGAAATAGACAGATCGCTGAGATAGGTGCCTCACTGATTAAGCATTGGTAACTGTCAGA
CCAAGTTTACTCATATATACTTTAGATTGATTTAAAACTTCATTTTTAATTTAAAAGGATCTAGGTGA
AGATCCTTTTTGATAATCTCATGACCAAAATCCCTTAACGTGAGTTTTCGTTCCACTGAGCGTCAG
ACCCCGTAGAAAAGATCAAAGGATCTTCTTGAGATCCTTTTTTTCTGCGCGTAATCTGCTGCTTGC
AAACAAAAAAACCACCGCTACCAGCGGTGGTTTGTTTGCCGGATCAAGAGCTACCAACTCTTTTT
CCGAAGGTAACTGGCTTCAGCAGAGCGCAGATACCAAATACTGTCCTTCTAGTGTAGCCGTAGTT
AGGCCACCACTTCAAGAACTCTGTAGCACCGCCTACATACCTCGCTCTGCTAATCCTGTTACCAG
TGGCTGCTGCCAGTGGCGATAAGTCGTGTCTTACCGGGTTGGACTCAAGACGATAGTTACCGGA
TAAGGCGCAGCGGTCGGGCTGAACGGGGGGTTCGTGCACACAGCCCAGCTTGGAGCGAACGA
CCTACACCGAACTGAGATACCTACAGCGTGAGCTATGAGAAAGCGCCACGCTTCCCGAAGGGAG
AAAGGCGGACAGGTATCCGGTAAGCGGCAGGGTCGGAACAGGAGAGCGCACGAGGGAGCTTC
CAGGGGGAAACGCCTGGTATCTTTATAGTCCTGTCGGGTTTCGCCACCTCTGACTTGAGCGTCG
ATTTTTGTGATGCTCGTCAGGGGGGCGGAGCCTATGGAAAAACGCCAGCAACGCGAGCTCGCG
ATCGCTTAATTAACGTTACATAACTTACGGTAAATGGCCCGCCTGGCTGACCGCCCAACGACCCC
CGCCCATTGACGTCAATAATGACGTATGTTCCCATAGTAACGCCAATAGGGACTTTCCATTGACG
TCAATGGGTGGAGTATTTACGGTAAACTGCCCACTTGGCAGTACATCAAGTGTATCATATGCCAA
GTACGCCCCCTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCATGGTGATGCG
GTTTTGGCAGTACATCAATGGGCGTGGATAGCGGTTTGACTCACGGGGATTTCCAAGTCTCCAC
CCCATTGACGTCAATGGGAGTTTGTTTTGGCACCAAAATCAACGGGACTTTCCAAAATGTCGTAA
CAACTCCGCCCCATTGACGCAAATGGGGGGTAGGCGTGTACGGTGGGAGGTCTATATAAGCAG
AGCTTAATACGACTCACTATAGGGCCGGCCATGGGCGGCGCATGAGAGAAGCCCAGACCAATTA
CCTACCCAAAATGGAGAAAGTTCACGTTGACATCGAGGAAGACAGCCCATTCCTCAGAGCTTTG
CAGCGGAGCTTCCCGCAGTTTGAGGTAGAAGCCAAGCAGGTCACTGATAATGACCATGCTAATG
CCAGAGCGTTTTCGCATCTGGCTTCAAAACTGATCGAAACGGAGGTGGACCCATCCGACACGAT
CCTTGACATTGGAAGTGCGCCCGCCCGCAGAATGTATTCTAAGCACAAGTATCATTGTATCTGTC
CGATGAGATGTGCGGAAGATCCGGACAGATTGTATAAGTATGCAACTAAGCTGAAGAAAAACTGT
AAGGAAATAACTGATAAGGAATTGGACAAGAAAATGAAGGAGCTGGCCGCCGTCATGAGCGACC
CTGACCTGGAAACTGAGACTATGTGCCTCCACGACGACGAGTCGTGTCGCTACGAAGGGCAAGT
CGCTGTTTACCAGGATGTATACGCGGTTGACGGACCGACAAGTCTCTATCACCAAGCCAATAAG
GGAGTTAGAGTCGCCTACTGGATAGGCTTTGACACCACCCCTTTTATGTTTAAGAACTTGGCTGG
AGCATATCCATCATACTCTACCAACTGGGCCGACGAAACCGTGTT
Further vector sequences
CMV+T7_VEE_GFP_NEW (Basic vector) (SEQ ID NO: 16)
GFP part can be replaced by another of gene of interest. VEE is modified and improved for the
enhanced protein expression
atgctctagactcctgcaggtaagtgtttaaaccgatgaatacagcagcaattggcaagctgcttacatagaactcgcggcgattggcatgccg
ctttaaaatttttattttatttttcttttcttttccgaatcggattttgtttttaatatttcaaaaaaaaaaaaaaaaaaaaaaaaaaacgcgtggccggca
tggtcccagcctcctcgctggcgccggctgggcaacatgcttcggcatggcgaatgggacctgtgccttctagttgccagccatctgttgtttgcc
cctcccccgtgccttccttgaccctggaaggtgccactcccactgtcctttcctaataaaatgaggaaattgcatcgcattgtctgagtaggtgtcat
tctattctggggggtggggggggcaggacagcaagggggaggattgggaagacaatagcaggcatgctggggatgcggtgggctctatg
gcgaggggaattaattcttgaagacgaaagggccaggtggcacttttcggggaaatgtgggccggcccgcggaacccctatttgtttatttttcta
aatacattcaaatatgtatccgctcatgagacaataaccctgataaatgcttcaataatattgaaaaaggaagagtatgagtattcaacatttccg
tgtcgcccttattcccttttttgcggcattttgccttcctgtttttgctcacccagaaacgctggtgaaagtaaaagatgctgaagatcagttgggtgca
cgagtgggttacatcgaactggatctcaacagcggtaagatccttgagagttttcgccccgaagaacgttttccaatgatgagcacttttaaagtt
ctgctatgtggcgcggtattatcccgtgttgacgccgggcaagagcaactcggtcgccgcatacactattctcagaatgacttggttgagtactca
ccagtcacagaaaagcatcttacggatggcatgacagtaagagaattatgcagtgctgccataaccatgagtgataacactgcggccaactt
acttctgacaacgatcggaggaccgaaggagctaaccgcttttttgcacaacatgggggatcatgtaactcgccttgatcgttgggaaccgga
gctgaatgaagccataccaaacgacgagcgtgacaccacgatgcctgtagcaatggcaacaacgttgcgcaaactattaactggcgaacta
cttactctagcttcccggcaacaattaatagactggatggaggcggataaagttgcaggaccacttctgcgctcggcccttccggctggctggttt
attgctgataaatctggagccggtgagcgtggatctcgcggtatcattgcagcactggggccagatggtaagccctcccgtatcgtagttatcta
cacgacggggagtcaggcaactatggatgaacgaaatagacagatcgctgagataggtgcctcactgattaagcattggtaactgtcagacc
aagtttactcatatatactttagattgatttaaaacttcatttttaatttaaaaggatctaggtgaagatcctttttgataatctcatgaccaaaatccctta
acgtgagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgcagata
ccaaatactgtccttctagtgtagccgtagttaggccaccacttcaagaactctgtagcaccgcctacatacctcgctctgctaatcctgttaccag
tggctgctgccagtggcgataagtcgtgtcttaccgggttggactcaagacgatagttaccggataaggcgcagcggtcgggctgaacgggg
ggttcgtgcacacagcccagcttggagcgaacgacctacaccgaactgagatacctacagcgtgagctatgagaaagcgccacgcttcccg
aagggagaaaggcggacaggtatccggtaagcggcagggtcggaacaggagagcgcacgagggagcttccagggggaaacgcctgg
tatctttatagtcctgtcgggtttcgccacctctgacttgagcgtcgatttttgtgatgctcgtcaggggggcggagcctatggaaaaacgccagca
acgcgagctcgcgatcgctcaatattggccattagccatattattcattggttatatagcataaatcaatattggctattggccattgcatacgttgtat
ctatatcataatatgtacatttatattggctcatgtccaatatgaccgccatgttggcattgattattgactagttattaatagtaatcaattacggggtc
attagttcatagcccatatatggagttccgcgttacataacttacggtaaatggcccgcctggctgaccgcccaacgacccccgcccattgacgt
caataatgacgtatgttcccatagtaacgccaatagggactttccattgacgtcaatgggtggagtatttacggtaaactgcccacttggcagtac
atcaagtgtatcatatgccaagtccgccccctattgacgtcaatgacggtaaatggcccgcctggcattatgcccagtacatgaccttacggga
ctttcctacttggcagtacatctacgtattagtcatcgctattaccatggtgatgcggttttggcagtacaccaatgggcgtggatagcggtttgactc
acggggatttccaagtctccaccccattgacgtcaatgggagtttgttttggcaccaaaatcaacgggactttccaaaatgtcgtaataaccccg
ccccgttgacgcaaatgggcggtaggcgtgtacggtgggaggtctatataagcagagctcgtttagtgaaccgtcagatcactagaagctttatt
gcggtagtttatcacagttaaattgctaacgcagtcagtgcttctgacacaacagtctcgaacttaagctgcagaagttggtcgtgaggcactgg
gcaggtaagtatcaaggttacaagacaggtttaaggagaccaatagaaactgggcttgtcgagacagagaagactcttgcgtttctgataggc
acctattggtcttactgacatccactttgcctttctctccacaggtgtccactcccagttcaattacagctcttaaggctagagtacttaatacgactca
ctataaggttaattaaatgggcggcgatgagagaagcccagaccaattacctacccaaaatggagaaagttcacgttgacatcgaggaaga
cagcccattcctcagagctttgcagcggagcttcccgcagtttgaggtagaagccaagcaggtcactgataatgaccatgctaatgccagagc
gttttcgcatctggcttcaaaactgatcgaaacggaggtggacccatccgacacgatccttgacattggaagtgcgcccgcccgcagaatgtat
tctaagcacaagtatcattgtatctgtccgatgagatgtgcggaagatccggacagattgtataagtatgcaactaagctgaagaaaaactgta
aggaaataactgataaggaattggacaagaaaatgaaggagctggccgccgtcatgagcgaccctgacctggaaactgagactatgtgcc
tccacgacgacgagtcgtgtcgctacgaagggcaagtcgctgtttaccaggatgtatacgcggttgacggaccgacaagtctctatcaccaag
ccaataagggagttagagtcgcctactggataggctttgacaccaccccttttatgtttaagaacttggctggagcatatccatcatactctaccaa
ctgggccgacgaaaccgtgttaacggctcgtaacataggcctatgcagctctgacgttatggagcggtcacgtagagggatgtccattcttaga
aagaagtatttgaaaccatccaacaatgttctattctctgttggctcgaccatctaccacgagaagagggacttactgaggagctggcacctgcc
gtctgtatttcacttacgtggcaagcaaaattacacatgtcggtgtgagactatagttagttgcgacgggtacgtcgttaaaagaatagctatcagt
ccaggcctgtatgggaagccttcaggctatgctgctacgatgcaccgcgagggattcttgtgctgcaaagtgacagacacattgaacgggga
gagggtatcttttcccgtgtgcacgtatgtgccagctacattgtgtgaccaaatgactggcatactggcaacagatgtcagtgcggacgacgcg
caaaaactgctggttgggctcaaccagcgtatagtcgtcaacggtcgcacccagagaaacaccaataccatgaaaaattaccttttgcccgta
gtggcccaggcatttgctaggtgggcaaaggaatataaggaagatcaagaagatgaaaggccactaggactacgagatagacagttagtc
atggggtgttgttgggcttttagaaggcacaagataacatctatttataagcgcccggatacccaaaccatcatcaaagtgaacagcgatttcca
ctcattcgtgctgcccaggataggcagtaacacattggagatcgggctgagaacaagaatcaggaaaatgttagaggagcacaaggagcc
gtcacctctcattaccgccgaggacgtacaagaagctaagtgcgcagccgatgaggctaaggaggtgcgtgaagccgaggagttgcgcgc
agctctaccacctttggcagctgatgttgaggagcccactctggaggcagacgtcgacttgatgttacaagaggctggggccggctcagtgga
gacacctcgtggcttgataaaggttaccagctacgatggcgaggacaagatcggctcttacgctgtgctttctccgcaggctgtactcaagagtg
aaaaattatcttgcatccaccctctcgctgaacaagtcatagtgataacacactctggccgaaaagggcgttatgccgtggaaccataccatgg
taaagtagtggtgccagagggacatgcaatacccgtccaggactttcaagctctgagtgaaagtgccaccattgtgtacaacgaacgtgagtt
cgtaaacaggtacctgcaccatattgccacacatggaggagcgctgaacactgatgaagaatattacaaaactgtcaagcccagcgagcac
gacggcgaatacctgtacgacatcgacaggaaacagtgcgtcaagaaagaactagtcactgggctagggctcacaggcgagctggtggat
cctcccttccatgaattcgcctacgagagtctgagaacacgaccagccgctccttaccaagtaccaaccataggggtgtatggcgtgccagga
tcaggcaagtctggcatcattaaaagcgcagtcaccaaaaaagatctagtggtgagcgccaagaaagaaaactgtgcagaaattataagg
gacgtcaagaaaatgaaagggctggacgtcaatgccagaactgtggactcagtgctcttgaatggatgcaaacaccccgtagaaaccctgt
atattgacgaagcttttgcttgtcatgcaggtactctcagagcgctcatagccattataagacctaaaaaggcagtgctctgcggggatcccaaa
cagtgcggtttttttaacatgatgtgcctgaaagtgcattttaaccacgagatttgcacacaagtcttccacaaaagcatctctcgccgttgcactaa
atctgtgacttcggttgtctcaaccttgttttacgacaaaaaaatgagaacgacgaatccgaaagagactaagattgtgattgacactaccggca
gtaccaaacctaagcaggacgatctcattctcacttgtttcagaggggggtgaagcagttgcaaatagattacaaaggcaacgaaataatga
cggcagctgcctctcaagggctgacccgtaaaggtgtgtatgccgttcggtacaaggtgaatgaaaatcctctgtacgcacccacctcagaac
atgtgaacgtcctactgacccgcacggaggaccgcatcgtgtggaaaacactagccggcgacccatggataaaaacactgactgccaagt
accctgggaatttcactgccacgatagaggagtggcaagcagagcatgatgccatcatgaggcacatcttggaaagaccggaccctaccga
cgtcttccagaataaggcaaacgtgtgttgggccaaggctttagtgccggtgctgaagaccgctggcatagacatgaccactgaacaatgga
acactgtggattattttgaaacggacaaagctcactcagcagagatagtattgaaccaactatgcgtgaggttctttggactcgatctggactccg
gtctattttctgcacccactgttccgttatccattaggaataatcactgggataactccccgtcgcctaacatgtacgggctgaataaagaagtggt
ccgtcagctctctcgcaggtacccacaactgcctcgggcagttgccactggaagagtctatgacatgaacactggtacactgcgcaattatgat
ccgcgcataaacctagtacctgtaaacagaagactgcctcatgctttagtcctccaccataatgaacacccacagagtgacttttcttcattcgtc
agcaaattgaagggcagaactgtcctggtggtcggggaaaagttgtccgtcccaggcaaaatggttgactggttgtcagaccggcctgaggct
accttcagagctcggctggatttaggcatcccaggtgatgtgcccaaatatgacataatatttgttaatgtgaggaccccatataaataccatcac
tatcagcagtgtgaagaccatgccattaagcttagcatgttgaccaagaaagcttgtctgcatctgaatcccggcggaacctgtgtcagcatag
gttatggttacgctgacagggccagcgaaagcatcattggtgctatagcgcggcagttcaagttttcccgggtatgcaaaccgaaatcctcactt
gaagaaacggaagttctgtttgtattcattgggtacgatcgcaaggcccgtacgcacaattcttacaagctttcatcaaccttgaccaacatttata
caggttccagactccacgaagccggatgtgcaccctcatatcatgtggtgcgaggggatattgccacggccaccgaaggagtgattataaat
gctgctaacagcaaaggacaacctggcggaggggtgtgcggagcgctgtataagaaattcccggaaagcttcgatttacagccgatcgaag
taggaaaagcgcgactggtcaaaggtgcagctaaacatatcattcatgccgtaggaccaaacttcaacaaagtttcggaggttgaaggtgac
aaacagttggcagaggcttatgagtccatcgctaagattgtcaacgataacaattacaagtcagtagcgattccactgttgtccaccggcatcttt
tccgggaacaaagatcgactaacccaatcattgaaccatttgctgacagctttagacaccactgatgcagatgtagccatatactgcagggac
aagaaatgggaaatgactctcaaggaagcagtggctaggagagaagcagtggaggagatatgcatatccgacgactcttcagtgacagaa
cctgatgcagagctggtgagggtgcatccgaagagttctttggctggaaggaagggctacagcacaagcgatggcaaaactttctcatatttg
gaagggaccaagtttcaccaggcggccaaggatatagcagaaattaatgccatgtggcccgttgcaacggaggccaatgagcaggtatgc
atgtatatcctcggagaaagcatgagcagtattaggtcgaaatgccccgtcgaagagtcggaagcctccacaccacctagcacgctgccttg
cttgtgcatccatgccatgactccagaaagagtacagcgcctaaaagcctcacgtccagaacaaattactgtgtgctcatcctttccattgccga
agtatagaatcactggtgtgcagaagatccaatgctcccagcctatattgttctcaccgaaagtgcctgcgtatattcatccaaggaagtatctcg
tggaaacaccaccggtagacgagactccggagccatcggcagagaaccaatccacagaggggacacctgaacaaccaccacttataac
cgaggatgagactaggactagaacgcctgagccgatcatcatcgaagaggaagaagaggatagcataagtttgctgtcagatggcccgac
ccaccaggtgctgcaagtcgaggcagacattcacgggccgccctctgtatctagctcatcctggtccattcctcatgcatccgactttgatgtgga
cagtttatccatacttgacaccctggagggagctagcgtgaccagcggggcaacgtcagccgagactaactcttacttcgcaaagagtatgga
gtttctggcgcgaccggtgcctgcgcctcgaacagtattcaggaaccctccacatcccgctccgcgcacaagaacaccgtcacttgcaccca
gcagggcctgctcgagaaccagcctagtttccaccccgccaggcgtgaatagggtgatcactagagaggagctcgaggcgcttaccccgtc
acgcactcctagcaggtcggtatcgagaaccagcctggtatccaacccgccaggcgtaaatagggtgattacaagagaggagtttgaggcg
ttcgtagcacaacaacaatgacggtttgatgcgggtgcatacatcttttcctccgacaccggtcaagggcatttacaacaaaaatcagtaaggc
aaacggtgctatccgaagtggtgttggagaggaccgaattggagatttcgtatgccccgcgcctcgaccaagaaaaagaagaattactacgc
aagaaattacagttaaatcccacacctgctaacagaagcagataccagtccaggaaggtggagaacatgaaagccataacagctagacgt
attctgcaaggcctagggcattatttgaaggcagaaggaaaagtggagtgctaccgaaccctgcatcctgttcctttgtattcatctagtgtgaac
cgtgccttttcaagccccaaggtcgcagtggaagcctgtaacgccatgttgaaagagaactttccgactgtggcttcttactgtattattccagagt
acgatgcctatttggacatggttgacggagcttcatgctgcttagacactgccagtttttgccctgcaaagctgcgcagctttccaaagaaacact
cctatttggaacccacaatacgatcggcagtgccttcagcgatccagaacacgctccagaacgtcctggcagctgccacaaaaagaaattgc
aatgtcacgcaaatgagagaattgcccgtattggattcggcggcctttaatgtggaatgcttcaagaaatatgcgtgtaataatgaatattggga
aacgtttaaagaaaaccccatcaggcttactgaagaaaacgtggtaaattacattaccaaattaaaaggaccaaaagctgctgctctttttgcg
aagacacataatttgaatatgttgcaggacataccaatggacaggtttgtaatggacttaaagagggacgtgaaagtgactccaggaacaaa
acatactgaagaacggcccaaggtacaggtgatccaggctgccgatccgctagcaacagcgtatctgtgcggaatccaccgagagctggtt
aggagattaaatgcggtcctgcttccgaacattcatacactgtttgatatgtcggctgaagactttgacgctattatagccgagcacttccagcctg
gggattgtgttctggaaactgacatcgcgtcgtttgataaaagtgaggacgacgccatggctctgaccgcgttaatgattctggaagacttaggt
gtggacgcagagctgttgacgctgattgaggcggctttcggcgaaatttcatcaatacatttgcccactaaaactaaatttaaattcggagccatg
atgaaatctggaatgttcctcacactgtttgtgaacacagtcattaacattgtaatcgcaagcagagtgttgagagaacggctaaccggatcacc
atgtgcagcattcattggagatgacaatatcgtgaaaggagtcaaatcggacaaattaatggcagacaggtgcgccacctggttgaatatgga
agtcaagattatagatgctgtggtgggcgagaaagcgccttatttctgtggagggtttattttgtgtgactccgtgaccggcacagcgtgccgtgtg
gcagaccccctaaaaaggctgtttaagcttggcaaacctctggcagcagacgatgaacatgatgatgacaggagaagggcattgcatgaag
agtcaacacgctggaaccgagtgggtattctttcagagctgtgcaaggcagtagaatcaaggtatgaaaccgtaggaacttccatcatagttat
ggccatgactactctagctagcagtgttaaatcattcagctacctgagaggggcccctataactctctacggctaacctgaatataggcggcgc
atgagagaagcccagaccaattacctacccaaagcaccatggagaaagttcacgttgacatcgaggaagacagcccattcctcagagcttt
gcagcggagcttcccgcagtttgaggtagaagccaagcaggtcactgatattgaccttgctaaagccagagcgttttcgcatctggctggcagc
ggcgcgcccgccacgaacttctctctgttaaagcaagcaggagacgtggaagaaaaccccggtcctatggtgagcaagggcgaggagctg
ttcaccggggtggtgcccatcctggtcgagctggacggcgacgtaaacggccacaagttcagcgtgtccggcgagggcgagggcgatgcc
acctacggcaagctgaccctgaagttcatctgcaccaccggcaagctgcccgtgccctggcccaccctcgtgaccaccctgacctacggcgt
gcagtgcttcagccgctaccccgaccacatgaagcagcacgacttcttcaagtccgccatgcccgaaggctacgtccaggagcgcaccatct
tcttcaaggacgacggcaactacaagacccgcgccgaggtgaagttcgagggcgacaccctggtgaaccgcatcgagctgaagggcatc
gacttcaaggaggacggcaacatcctggggcacaagctggagtacaactacaacagccacaacgtctatatcatggccgacaagcagaa
gaacggcatcaaggtgaacttcaagatccgccacaacatcgaggacggcagcgtgcagctcgccgaccactaccagcagaacaccccc
atcggcgacggccccgtgctgctgcccgacaaccactacctgagcacccagtccgccctgagcaaagaccccaacgagaagcgcgatca
catggtcctgctggagttcgtgaccgccgccgggatcactctcggcatggacgagctgtacaagt

| Feature list | | | | |
|---|---|---|---|---|
| source | 1 . . . 11,873 | 11,873 | == | source |
| ATGC | 1 . . . 4 | 4 | == | homology |
| XbaI restriction site | 5 . . . 10 | 6 | == | misc_feature |
| source (part) | 11 . . . 12 | 2 | == | source |
| source | 11 . . . 12 | 2 | == | source |
| CT dinucleotide | 11 . . . 12 | 2 | == | misc_feature |
| source | 13 . . . 20 | 8 | == | source |
| source | 13 . . . 20 | 8 | == | source |
| source | 13 . . . 20 | 8 | == | source |
| SbfI restriction site | 13 . . . 20 | 8 | == | misc_feature |
| STOP | 21 . . . 23 | 3 | == | misc_feature |
| 3′ UTR | 37 . . . 156 | 120 | == | 3′UTR |
| HDV ribozyme | 189 . . . 256 | 68 | => | ncRNA |
| bGH poly(A) signal | 257 . . . 481 | 225 | == | polyA_signal |
| AmpR promoter (part) | 548 . . . 652 | 105 | == | promoter |
| AmpR (part) | 653 . . . 721 | 69 | => | CDS |
| ATCA | 1116 . . . 1119 | 4 | == | homology |
| G −> A (part) | 1369 . . . 1369 | 1 | == | misc_feature |
| ori (part) | 1684 . . . 2272 | 589 | => | rep_origin |
| source | 2293 . . . 2300 | 8 | == | source |
| AsiSI restriction site | 2293 . . . 2300 | 8 | == | misc_feature |
| CMV enhancer | 2438 . . . 2817 | 380 | == | enhancer |
| CMV promoter | 2818 . . . 3021 | 204 | == | promoter |
| chimeric intron | 3157 . . . 3289 | 133 | == | intron |
| T7 promoter (part) | 3334 . . . 3353 | 20 | == | promoter |
| G > A | 3351 . . . 3351 | 1 | == | misc_feature |
| Corrected the nt back to original | 3364 . . . 3364 | 1 | == | misc_feature |
| left_flank | 4215 . . . 4215 | 1 | == | misc_feature |
| C −> A (part) | 4382 . . . 4382 | 1 | == | misc_feature |
| Shine-Dalgarno sequence (part) | 4895 . . . 4903 | 9 | == | RBS |
| AGAA | 5747 . . . 5750 | 4 | == | homology |
| C −> T (part) | 5966 . . . 5966 | 1 | == | misc_feature |
| G −> A (part) | 6395 . . . 6395 | 1 | == | misc_feature |
| G −> A (part) | 7271 . . . 7271 | 1 | == | misc_feature |
| C −> T (part) | 8483 . . . 8483 | 1 | == | misc_feature |
| C −> A (part) | 8954 . . . 8954 | 1 | == | misc_feature |
| C −> A (part) | 8972 . . . 8972 | 1 | == | misc_feature |
| A −> G (part) | 9935 . . . 9935 | 1 | == | misc_feature |
| ACAG | 10,017 . . . 10,020 | 4 | == | homology |
| 26S Subgenomic promoter (part) | 10,873 . . . 10,893 | 21 | == | misc_feature |
| 5′UTR_Second | 10,894 . . . 10,937 | 44 | == | misc_feature |
| Auxilary Seq | 10,944 . . . 11,083 | 140 | == | misc_feature |
| 51 nt CSE(NSP1) | 11,031 . . . 11,082 | 52 | == | misc_feature |
| P2A | 11,099 . . . 11,155 | 57 | => | CDS |
| source | 11,156 . . . 11,872 | 717 | == | source |
| enhanced GFP | 11,156 . . . 11,872 | 717 | => | CDS |
| EGFP, reverse primer | 11,201 . . . 11,222 | 22 | <= | primer_bind |
| For distinguishing EGFP variants, reverse primer | 11,462 . . . 11,481 | 20 | <= | primer_bind |
| EGFP, forward primer | 11,809 . . . 11,830 | 22 | => | primer_bind |

CMV + T7_VEE_GFP_HBA
(SEQ ID NO: 17)
This vector has additional 5′ and 3′ UTR from HBA1
atgctctagactcctgcaggtaagtgtttaaaccgatgaatacagcagcaattggcaagctgcttacatagaactcgcggcgattggcatgccg
ctttaaaatttttattttatttttcttttcttttccgaatcggattttgtttttaatatttcaaaaaaaaaaaaaaaaaaaaaaaaaaacgcgtggccggca
tggtcccagcctcctcgctggcgccggctgggcaacatgcttcggcatggcgaatgggacaataaagtctgagtgggcggcacgaggggaa
ttaattcttgaagacgaaagggccaggtggcacttttcggggaaatgtgggccggcccgcggaacccctatttgtttatttttctaaatacattcaa
atatgtatccgctcatgagacaataaccctgataaatgcttcaataatattgaaaaaggaagagtatgagtattcaacatttccgtgtcgcccttat
tcccttttttgcggcattttgccttcctgtttttgctcacccagaaacgctggtgaaagtaaaagatgctgaagatcagttgggtgcacgagtgggtta
catcgaactggatctcaacagcggtaagatccttgagagttttcgccccgaagaacgttttccaatgatgagcacttttaaagttctgctatgtggc
gcggtattatcccgtgttgacgccgggcaagagcaactcggtcgccgcatacactattctcagaatgacttggttgagtactcaccagtcacag
aaaagcatcttacggatggcatgacagtaagagaattatgcagtgctgccataaccatgagtgataacactgcggccaacttacttctgacaa
cgatcggaggaccgaaggagctaaccgcttttttgcacaacatgggggatcatgtaactcgccttgatcgttgggaaccggagctgaatgaag
ccataccaaacgacgagcgtgacaccacgatgcctgtagcaatggcaacaacgttgcgcaaactattaactggcgaactacttactctagctt
cccggcaacaattaatagactggatggaggcggataaagttgcaggaccacttctgcgctcggcccttccggctggctggtttattgctgataa
atctggagccggtgagcgtggatctcgcggtatcattgcagcactggggccagatggtaagccctcccgtatcgtagttatctacacgacgggg
agtcaggcaactatggatgaacgaaatagacagatcgctgagataggtgcctcactgattaagcattggtaactgtcagaccaagtttactcat
atatactttagattgatttaaaacttcatttttaatttaaaaggatctaggtgaagatcctttttgataatctcatgaccaaaatcccttaacgtgagttttc
gttccactgagcgtcagaccccgtagaaaagatcaaaggatcttcttgagatcctttttttctgcgcgtaatctgctgcttgcaaacaaaaaaacc
accgctaccagcggtggtttgtttgccggatcaagagctaccaactctttttccgaaggtaactggcttcagcagagcgcagataccaaatactg
tccttctagtgtagccgtagttaggccaccacttcaagaactctgtagcaccgcctacatacctcgctctgctaatcctgttaccagtggctgctgc
cagtggcgataagtcgtgtcttaccgggttggactcaagacgatagttaccggataaggcgcagcggtcgggctgaacggggggttcgtgca
cacagcccagcttggagcgaacgacctacaccgaactgagatacctacagcgtgagctatgagaaagcgccacgcttcccgaagggaga
aaggcggacaggtatccggtaagcggcagggtcggaacaggagagcgcacgagggagcttccagggggaaacgcctggtatctttatag
tcctgtcgggtttcgccacctctgacttgagcgtcgatttttgtgatgctcgtcaggggggcggagcctatggaaaaacgccagcaacgcgagct
cgcgatcgctcaatattggccattagccatattattcattggttatatagcataaatcaatattggctattggccattgcatacgttgtatctatatcata
atatgtacatttatattggctcatgtccaatatgaccgccatgttggcattgattattgactagttattaatagtaatcaattacggggtcattagttcata
gcccatatatggagttccgcgttacataacttacggtaaatggcccgcctggctgaccgcccaacgacccccgcccattgacgtcaataatga
cgtatgttcccatagtaacgccaatagggactttccattgacgtcaatgggtggagtatttacggtaaactgcccacttggcagtacatcaagtgt
atcatatgccaagtccgccccctattgacgtcaatgacggtaaatggcccgcctggcattatgcccagtacatgaccttacgggactttcctactt
ggcagtacatctacgtattagtcatcgctattaccatggtgatgcggttttggcagtacaccaatgggcgtggatagcggtttgactcacggggatt
tccaagtctccaccccattgacgtcaatgggagtttgttttggcaccaaaatcaacgggactttccaaaatgtcgtaataaccccgccccgttgac
gcaaatgggcggtaggcgtgtacggtgggaggtctatataagcagagctcgtttagtgaaccgtcagatcactagaagctttattgcggtagttt
atcacagttaaattgctaacgcagtcagtgcttctgacacaacagtctcgaacttaagctgcagaagttggtcgtgaggcactgggcaggtaag
tatcaaggttacaagacaggtttaaggagaccaatagaaactgggcttgtcgagacagagaagactcttgcgtttctgataggcacctattggt
cttactgacatccactttgcctttctctccacaggtgtccactcccagttcaattacagctcttaaggctagagtacttaatacgactcactataaggtt
aattaaatgggcggcgatgagagaagcccagaccaattacctacccaaaatggagaaagttcacgttgacatcgaggaagacagcccatt
cctcagagctttgcagcggagcttcccgcagtttgaggtagaagccaagcaggtcactgataatgaccatgctaatgccagagcgttttcgcat
ctggcttcaaaactgatcgaaacggaggtggacccatccgacacgatccttgacattggaagtgcgcccgcccgcagaatgtattctaagca
caagtatcattgtatctgtccgatgagatgtgcggaagatccggacagattgtataagtatgcaactaagctgaagaaaaactgtaaggaaat
aactgataaggaattggacaagaaaatgaaggagctggccgccgtcatgagcgaccctgacctggaaactgagactatgtgcctccacga
cgacgagtcgtgtcgctacgaagggcaagtcgctgtttaccaggatgtatacgcggttgacggaccgacaagtctctatcaccaagccaataa
gggagttagagtcgcctactggataggctttgacaccaccccttttatgtttaagaacttggctggagcatatccatcatactctaccaactgggcc
gacgaaaccgtgttaacggctcgtaacataggcctatgcagctctgacgttatggagcggtcacgtagagggatgtccattcttagaaagaagt
atttgaaaccatccaacaatgttctattctctgttggctcgaccatctaccacgagaagagggacttactgaggagctggcacctgccgtctgtatt
tcacttacgtggcaagcaaaattacacatgtcggtgtgagactatagttagttgcgacgggtacgtcgttaaaagaatagctatcagtccaggcc
tgtatgggaagccttcaggctatgctgctacgatgcaccgcgagggattcttgtgctgcaaagtgacagacacattgaacggggagagggtat
cttttcccgtgtgcacgtatgtgccagctacattgtgtgaccaaatgactggcatactggcaacagatgtcagtgcggacgacgcgcaaaaact
gctggttgggctcaaccagcgtatagtcgtcaacggtcgcacccagagaaacaccaataccatgaaaaattaccttttgcccgtagtggccca
ggcatttgctaggtgggcaaaggaatataaggaagatcaagaagatgaaaggccactaggactacgagatagacagttagtcatggggtgt
tgttgggcttttagaaggcacaagataacatctatttataagcgcccggatacccaaaccatcatcaaagtgaacagcgatttccactcattcgt
gctgcccaggataggcagtaacacattggagatcgggctgagaacaagaatcaggaaaatgttagaggagcacaaggagccgtcacctct
cattaccgccgaggacgtacaagaagctaagtgcgcagccgatgaggctaaggaggtgcgtgaagccgaggagttgcgcgcagctctac
cacctttggcagctgatgttgaggagcccactctggaggcagacgtcgacttgatgttacaagaggctggggccggctcagtggagacacctc
gtggcttgataaaggttaccagctacgatggcgaggacaagatcggctcttacgctgtgctttctccgcaggctgtactcaagagtgaaaaatta
tcttgcatccaccctctcgctgaacaagtcatagtgataacacactctggccgaaaagggcgttatgccgtggaaccataccatggtaaagtag
tggtgccagagggacatgcaatacccgtccaggactttcaagctctgagtgaaagtgccaccattgtgtacaacgaacgtgagttcgtaaaca
ggtacctgcaccatattgccacacatggaggagcgctgaacactgatgaagaatattacaaaactgtcaagcccagcgagcacgacggcg
aatacctgtacgacatcgacaggaaacagtgcgtcaagaaagaactagtcactgggctagggctcacaggcgagctggtggatcctcccttc
catgaattcgcctacgagagtctgagaacacgaccagccgctccttaccaagtaccaaccataggggtgtatggcgtgccaggatcaggca
agtctggcatcattaaaagcgcagtcaccaaaaaagatctagtggtgagcgccaagaaagaaaactgtgcagaaattataagggacgtca
agaaaatgaaagggctggacgtcaatgccagaactgtggactcagtgctcttgaatggatgcaaacaccccgtagaaaccctgtatattgac
gaagcttttgcttgtcatgcaggtactctcagagcgctcatagccattataagacctaaaaaggcagtgctctgcggggatcccaaacagtgcg
gtttttttaacatgatgtgcctgaaagtgcattttaaccacgagatttgcacacaagtcttccacaaaagcatctctcgccgttgcactaaatctgtg
acttcggttgtctcaaccttgttttacgacaaaaaaatgagaacgacgaatccgaaagagactaagattgtgattgacactaccggcagtacca
aacctaagcaggacgatctcattctcacttgtttcagaggggggtgaagcagttgcaaatagattacaaaggcaacgaaataatgacggca
gctgcctctcaagggctgacccgtaaaggtgtgtatgccgttcggtacaaggtgaatgaaaatcctctgtacgcacccacctcagaacatgtga
acgtcctactgacccgcacggaggaccgcatcgtgtggaaaacactagccggcgacccatggataaaaacactgactgccaagtaccctg
ggaatttcactgccacgatagaggagtggcaagcagagcatgatgccatcatgaggcacatcttggaaagaccggaccctaccgacgtcttc
cagaataaggcaaacgtgtgttgggccaaggctttagtgccggtgctgaagaccgctggcatagacatgaccactgaacaatggaacactgt
ggattattttgaaacggacaaagctcactcagcagagatagtattgaaccaactatgcgtgaggttctttggactcgatctggactccggtctatttt
ctgcacccactgttccgttatccattaggaataatcactgggataactccccgtcgcctaacatgtacgggctgaataaagaagtggtccgtcag
ctctctcgcaggtacccacaactgcctcgggcagttgccactggaagagtctatgacatgaacactggtacactgcgcaattatgatccgcgca
taaacctagtacctgtaaacagaagactgcctcatgctttagtcctccaccataatgaacacccacagagtgacttttcttcattcgtcagcaaatt
gaagggcagaactgtcctggtggtcggggaaaagttgtccgtcccaggcaaaatggttgactggttgtcagaccggcctgaggctaccttcag
agctcggctggatttaggcatcccaggtgatgtgcccaaatatgacataatatttgttaatgtgaggaccccatataaataccatcactatcagca
gtgtgaagaccatgccattaagcttagcatgttgaccaagaaagcttgtctgcatctgaatcccggcggaacctgtgtcagcataggttatggtta
cgctgacagggccagcgaaagcatcattggtgctatagcgcggcagttcaagttttcccgggtatgcaaaccgaaatcctcacttgaagaaac
ggaagttctgtttgtattcattgggtacgatcgcaaggcccgtacgcacaattcttacaagctttcatcaaccttgaccaacatttatacaggttcca
gactccacgaagccggatgtgcaccctcatatcatgtggtgcgaggggatattgccacggccaccgaaggagtgattataaatgctgctaac
agcaaaggacaacctggcggaggggtgtgcggagcgctgtataagaaattcccggaaagcttcgatttacagccgatcgaagtaggaaaa
gcgcgactggtcaaaggtgcagctaaacatatcattcatgccgtaggaccaaacttcaacaaagtttcggaggttgaaggtgacaaacagttg
gcagaggcttatgagtccatcgctaagattgtcaacgataacaattacaagtcagtagcgattccactgttgtccaccggcatcttttccgggaac
aaagatcgactaacccaatcattgaaccatttgctgacagctttagacaccactgatgcagatgtagccatatactgcagggacaagaaatgg
gaaatgactctcaaggaagcagtggctaggagagaagcagtggaggagatatgcatatccgacgactcttcagtgacagaacctgatgca
gagctggtgagggtgcatccgaagagttctttggctggaaggaagggctacagcacaagcgatggcaaaactttctcatatttggaagggac
caagtttcaccaggcggccaaggatatagcagaaattaatgccatgtggcccgttgcaacggaggccaatgagcaggtatgcatgtatatcct
cggagaaagcatgagcagtattaggtcgaaatgccccgtcgaagagtcggaagcctccacaccacctagcacgctgccttgcttgtgcatcc
atgccatgactccagaaagagtacagcgcctaaaagcctcacgtccagaacaaattactgtgtgctcatcctttccattgccgaagtatagaat
cactggtgtgcagaagatccaatgctcccagcctatattgttctcaccgaaagtgcctgcgtatattcatccaaggaagtatctcgtggaaacac
caccggtagacgagactccggagccatcggcagagaaccaatccacagaggggacacctgaacaaccaccacttataaccgaggatga
gactaggactagaacgcctgagccgatcatcatcgaagaggaagaagaggatagcataagtttgctgtcagatggcccgacccaccaggt
gctgcaagtcgaggcagacattcacgggccgccctctgtatctagctcatcctggtccattcctcatgcatccgactttgatgtggacagtttatcc
atacttgacaccctggagggagctagcgtgaccagcggggcaacgtcagccgagactaactcttacttcgcaaagagtatggagtttctggc
gcgaccggtgcctgcgcctcgaacagtattcaggaaccctccacatcccgctccgcgcacaagaacaccgtcacttgcacccagcagggc
ctgctcgagaaccagcctagtttccaccccgccaggcgtgaatagggtgatcactagagaggagctcgaggcgcttaccccgtcacgcactc
ctagcaggtcggtatcgagaaccagcctggtatccaacccgccaggcgtaaatagggtgattacaagagaggagtttgaggcgttcgtagca
caacaacaatgacggtttgatgcgggtgcatacatcttttcctccgacaccggtcaagggcatttacaacaaaaatcagtaaggcaaacggtg
ctatccgaagtggtgttggagaggaccgaattggagatttcgtatgccccgcgcctcgaccaagaaaaagaagaattactacgcaagaaatt
acagttaaatcccacacctgctaacagaagcagataccagtccaggaaggtggagaacatgaaagccataacagctagacgtattctgca
aggcctagggcattatttgaaggcagaaggaaaagtggagtgctaccgaaccctgcatcctgttcctttgtattcatctagtgtgaaccgtgccttt
tcaagccccaaggtcgcagtggaagcctgtaacgccatgttgaaagagaactttccgactgtggcttcttactgtattattccagagtacgatgcc
tatttggacatggttgacggagcttcatgctgcttagacactgccagtttttgccctgcaaagctgcgcagctttccaaagaaacactcctatttgga
acccacaatacgatcggcagtgccttcagcgatccagaacacgctccagaacgtcctggcagctgccacaaaaagaaattgcaatgtcacg
caaatgagagaattgcccgtattggattcggcggcctttaatgtggaatgcttcaagaaatatgcgtgtaataatgaatattgggaaacgtttaaa
gaaaaccccatcaggcttactgaagaaaacgtggtaaattacattaccaaattaaaaggaccaaaagctgctgctctttttgcgaagacacat
aatttgaatatgttgcaggacataccaatggacaggtttgtaatggacttaaagagggacgtgaaagtgactccaggaacaaaacatactga
agaacggcccaaggtacaggtgatccaggctgccgatccgctagcaacagcgtatctgtgcggaatccaccgagagctggttaggagatta
aatgcggtcctgcttccgaacattcatacactgtttgatatgtcggctgaagactttgacgctattatagccgagcacttccagcctggggattgtgt
tctggaaactgacatcgcgtcgtttgataaaagtgaggacgacgccatggctctgaccgcgttaatgattctggaagacttaggtgtggacgca
gagctgttgacgctgattgaggcggctttcggcgaaatttcatcaatacatttgcccactaaaactaaatttaaattcggagccatgatgaaatctg
gaatgttcctcacactgtttgtgaacacagtcattaacattgtaatcgcaagcagagtgttgagagaacggctaaccggatcaccatgtgcagc
attcattggagatgacaatatcgtgaaaggagtcaaatcggacaaattaatggcagacaggtgcgccacctggttgaatatggaagtcaagat
tatagatgctgtggtgggcgagaaagcgccttatttctgtggagggtttattttgtgtgactccgtgaccggcacagcgtgccgtgtggcagaccc
cctaaaaaggctgtttaagcttggcaaacctctggcagcagacgatgaacatgatgatgacaggagaagggcattgcatgaagagtcaaca
cgctggaaccgagtgggtattctttcagagctgtgcaaggcagtagaatcaaggtatgaaaccgtaggaacttccatcatagttatggccatga
ctactctagctagcagtgttaaatcattcagctacctgagaggggcccctataactctctacggctaacctgaatactcttctggtccccacagact
cagagagaacccaccataggcggcgcatgagagaagcccagaccaattacctacccaaagcaccatggagaaagttcacgttgacatcg
aggaagacagcccattcctcagagctttgcagcggagcttcccgcagtttgaggtagaagccaagcaggtcactgatattgaccttgctaaag
ccagagcgttttcgcatctggctggcagcggcgcgcccgccacgaacttctctctgttaaagcaagcaggagacgtggaagaaaaccccgg
tcctatggtgagcaagggcgaggagctgttcaccggggtggtgcccatcctggtcgagctggacggcgacgtaaacggccacaagttcagc
gtgtccggcgagggcgagggcgatgccacctacggcaagctgaccctgaagttcatctgcaccaccggcaagctgcccgtgccctggccc
accctcgtgaccaccctgacctacggcgtgcagtgcttcagccgctaccccgaccacatgaagcagcacgacttcttcaagtccgccatgccc
gaaggctacgtccaggagcgcaccatcttcttcaaggacgacggcaactacaagacccgcgccgaggtgaagttcgagggcgacaccct
ggtgaaccgcatcgagctgaagggcatcgacttcaaggaggacggcaacatcctggggcacaagctggagtacaactacaacagccaca
acgtctatatcatggccgacaagcagaagaacggcatcaaggtgaacttcaagatccgccacaacatcgaggacggcagcgtgcagctcg
ccgaccactaccagcagaacacccccatcggcgacggccccgtgctgctgcccgacaaccactacctgagcacccagtccgccctgagc
aaagaccccaacgagaagcgcgatcacatggtcctgctggagttcgtgaccgccgccgggatcactctcggcatggacgagctgtacaagt

| Feature list | | | | |
|---|---|---|---|---|
| source | 1 . . . 11,707 | 11,707 | == | source |
| ATGC | 1 . . . 4 | 4 | == | homology |
| XbaI restriction site | 5 . . . 10 | 6 | == | misc_feature |
| source (part) | 11 . . . 12 | 2 | == | source |
| source | 11 . . . 12 | 2 | == | source |
| CT dinucleotide | 11 . . . 12 | 2 | == | misc_feature |
| source | 13 . . . 20 | 8 | == | source |
| source | 13 . . . 20 | 8 | == | source |
| source | 13 . . . 20 | 8 | == | source |
| SbfI restriction site | 13 . . . 20 | 8 | == | misc_feature |
| STOP | 21 . . . 23 | 3 | == | misc_feature |
| 3′ UTR | 37 . . . 156 | 120 | == | 3′UTR |
| HDV ribozyme | 189 . . . 256 | 68 | => | ncRNA |
| HBA1 PolyA | 257 . . . 278 | 22 | == | misc_feature |
| AmpR promoter (part) | 345 . . . 449 | 105 | == | promoter |
| AmpR (part) | 450 . . . 518 | 69 | => | CDS |
| ATCA | 913 . . . 916 | 4 | == | homology |
| G −> A (part) | 1166 . . . 1166 | 1 | == | misc_feature |
| ori (part) | 1481 . . . 2069 | 589 | => | rep_origin |
| source | 2090 . . . 2097 | 8 | == | source |
| AsiSI restriction site | 2090 . . . 2097 | 8 | == | misc_feature |
| CMV enhancer | 2235 . . . 2614 | 380 | == | enhancer |
| CMV promoter | 2615 . . . 2818 | 204 | == | promoter |
| chimeric intron | 2954 . . . 3086 | 133 | == | intron |
| T7 promoter (part) | 3131 . . . 3150 | 20 | == | promoter |
| G > A | 3148 . . . 3148 | 1 | == | misc_feature |
| Corrected the nt back to original | 3161 . . . 3161 | 1 | == | misc_feature |
| left_flank | 4012 . . . 4012 | 1 | == | misc_feature |
| C −> A (part) | 4179 . . . 4179 | 1 | == | misc_feature |
| Shine-Dalgarno sequence (part) | 4692 . . . 4700 | 9 | == | RBS |
| AGAA | 5544 . . . 5547 | 4 | == | homology |
| C −> T (part) | 5763 . . . 5763 | 1 | == | misc_feature |
| G −> A (part) | 6192 . . . 6192 | 1 | == | misc_feature |
| G −> A (part) | 7068 . . . 7068 | 1 | == | misc_feature |
| C −> T (part) | 8280 . . . 8280 | 1 | == | misc_feature |
| C −> A (part) | 8751 . . . 8751 | 1 | == | misc_feature |
| C −> A (part) | 8769 . . . 8769 | 1 | == | misc_feature |
| A −> G (part) | 9732 . . . 9732 | 1 | == | misc_feature |
| ACAG | 9814 . . . 9817 | 4 | == | homology |
| 26S Subgenomic promoter (part) | 10,670 . . . 10,690 | 21 | == | misc_feature |
| HBA1-5UTR | 10,691 . . . 10,727 | 37 | == | misc_feature |
| 5′UTR_VEE | 10,728 . . . 10,771 | 44 | == | misc_feature |
| Auxilary Seq | 10,778 . . . 10,917 | 140 | == | misc_feature |
| 51 nt CSE(NSP1) | 10,865 . . . 10,916 | 52 | == | misc_feature |
| P2A | 10,933 . . . 10,989 | 57 | => | CDS |
| source | 10,990 . . . 11,706 | 717 | == | source |
| enhanced GFP | 10,990 . . . 11,706 | 717 | => | CDS |
| EGFP, reverse primer | 11,035 . . . 11,056 | 22 | <= | primer_bind |
| For distinguishing EGFP variants, reverse primer | 11,296 . . . 11,315 | 20 | <= | primer_bind |
| EGFP, forward primer | 11,643 . . . 11,664 | 22 | => | primer_bind |

CMV + T7_COV-2_GFP
(SEQ ID NO: 18)
atgctctagactcctgcaggGTTtaaacgaacatgggctatataaacgttttcgcttttccgtttacgatatatagtctactcttgtgcagaatgaat
tctcgtaactacatagcacaagtagatgtagttaactttaatctcacatagcaatctttaatcagtgtgtaacattagggaggacttgaaagagcc
accacattttcaccgaggccacgcggagtacgatcgagtgtacagtgaacaatgctagggagagctgcctatatggaagagccctaatgtgt
aaaattaattttagtagtgctatccccatgtgattttaatagcttcttaggagaatgacaaaaaaaaaaaaaaaaaaaaaaaaaaaaaaaaa
gcggccgcGGCCGGCATGGTCCCAGCCTCCTCGCTGGCGCCGGCTGGGCAACATGCTTCGGCATG
GCGAATGGGACCtgtgccttctagttgccagccatctgttgtttgcccctcccccgtgccttccttgaccctggaaggtgccactcccactgt
cctttcctaataaaatgaggaaattgcatcgcattgtctgagtaggtgtcattctattctggggggtggggggggcaggacagcaagggggag
gattgggaagacaatagcaggcatgctggggatgcggtgggctctatggcgcggaacccctatttgtttatttttctaaatacattcaaatatgtat
ccgctcatgagacaataaccctgataaatgcttcaataatattgaaaaaggaagagtatgagtattcaacatttccgtgtcgcccttattccctttttt
gcggcattttgccttcctgtttttgctcacccagaaacgctggtgaaagtaaaagatgctgaagatcagttgggtgcacgagtgggttacatcgaa
ctggatctcaacagcggtaagatccttgagagttttcgccccgaagaacgttttccaatgatgagcacttttaaagttctgctatgtggcgcggtatt
atcccgtgttgacgccgggcaagagcaactcggtcgccgcatacactattctcagaatgacttggttgagtactcaccagtcacagaaaagca
tcttacggatggcatgacagtaagagaattatgcagtgctgccataaccatgagtgataacactgcggccaacttacttctgacaacgatcgga
ggaccgaaggagctaaccgcttttttgcacaacatgggggatcatgtaactcgccttgatcgttgggaaccggagctgaatgaagccatacca
aacgacgagcgtgacaccacgatgcctgtagcaatggcaacaacgttgcgcaaactattaactggcgaactacttactctagcttcccggca
acaattaatagactggatggaggcggataaagttgcaggaccacttctgcgctcggcccttccggctggctggtttattgctgataaatctggag
ccggtgagcgtggctctcgcggtatcattgcagcactggggccagatggtaagccctcccgtatcgtagttatctacacgacggggagtcagg
caactatggatgaacgaaatagacagatcgctgagataggtgcctcactgattaagcattggtaactgtcagaccaagtttactcatatatacttt
agattgatttaaaacttcatttttaatttaaaaggatctaggtgaagatcctttttgataatctcatgaccaaaatcccttaacgtgagttttcgttccact
gagcgtcagaccccgtagaaaagatcaaaggatcttcttgagatcctttttttctgcgcgtaatctgctgcttgcaaacaaaaaaaccaccgcta
ccagcggtggtttgtttgccggatcaagagctaccaactctttttccgaaggtaactggcttcagcagagcgcagataccaaatactgtccttcta
gtgtagccgtagttaggccaccacttcaagaactctgtagcaccgcctacatacctcgctctgctaatcctgttaccagtggctgctgccagtggc
gataagtcgtgtcttaccgggttggactcaagacgatagttaccggataaggcgcagcggtcgggctgaacggggggttcgtgcacacagcc
cagcttggagcgaacgacctacaccgaactgagatacctacagcgtgagctatgagaaagcgccacgcttcccgaagggagaaaggcgg
acaggtatccggtaagcggcagggtcggaacaggagagcgcacgagggagcttccagggggaaacgcctggtatctttatagtcctgtcgg
gtttcgccacctctgacttgagcgtcgatttttgtgatgctcgtcaggggggcggagcctatggaaaaacgccagcaacgcgagctcgcgatcg
ctcaatattggccattagccatattattcattggttatatagcataaatcaatattggctattggccattgcatacgttgtatctatatcataatatgtaca
tttatattggctcatgtccaatatgaccgccatgttggcattgattattgactagttattaatagtaatcaattacggggtcattagttcatagcccatat
atggagttccgcgttacataacttacggtaaatggcccgcctggctgaccgcccaacgacccccgcccattgacgtcaataatgacgtatgttc
ccatagtaacgccaatagggactttccattgacgtcaatgggtggagtatttacggtaaactgcccacttggcagtacatcaagtgtatcatatgc
caagtccgccccctattgacgtcaatgacggtaaatggcccgcctggcattatgcccagtacatgaccttacgggactttcctacttggcagtac
atctacgtattagtcatcgctattaccatggtgatgcggttttggcagtacaccaatgggcgtggatagcggtttgactcacggggatttccaagtct
ccaccccattgacgtcaatgggagtttgttttggcaccaaaatcaacgggactttccaaaatgtcgtaataaccccgccccgttgacgcaaatg
ggcggtaggcgtgtacggtgggaggtctatataagcagagctcgtttagtgaaccgtcagatcactagaagctttattgcggtagtttatcacagt
taaattgctaacgcagtcagtgcttctgacacaacagtctcgaacttaagctgcagaagttggtcgtgaggcactgggcaggtaagtatcaagg
ttacaagacaggtttaaggagaccaatagaaactgggcttgtcgagacagagaagactcttgcgtttctgataggcacctattggtcttactgac
atccactttgcctttctctccacagGTGTCCACTCCCAGTTCAATTACAGCTCTTAAGGCTAGAGTACTtaatacga
ctcactataaggggccggccattaaaggtttataccttcccaggtaacaaaccaaccaactttcgatctcttgtagatctgttctctaaacgaacttt
aaaatctgtgtggctgtcactcggctgcatgcttagtgcactcacgcagtataattaataactaattactgtcgttgacaggacacgagtaactcgt
ctatcttctgcaggctgcttacggtttcgtccgtgttgcagccgatcatcagcacatctaggtttcgtccgggtgtgaccgaaaggtaagGCCA
CCatggagagccttgtccctggtttcaacgagaaaacacacgtccaactcagtttgcctgttttacaggttcgcgacgtgctcgtacgtggctttg
gagactccgtggaggaggtcttatcagaggcacgtcaacatcttaaagatggcacttgtggcttagtagaagttgaaaaaggcgttttgcctca
acttgaacagccctatgtgttcatcaaacgttcggatgctcgaactgcacctcatggtcatgttatggttgagctggtagcagaactcgaaggcat
tcagtacggtcgtagtggtgagacacttggtgtccttgtccctcatgtgggcgaaataccagtggcttaccgcaaggttcttcttcgtaagaacggt
aataaaggagctggtggccatagttacggcgccgatctaaagtcatttgacttaggcgacgagcttggcactgatccttatgaagattttcaaga
aaactggaacactaaacatagcagtggtgttacccgtgaactcatgcgtgagcttaacggaggggctgttttgcagagtggttttagaaaaatg
gcattcccatctggtaaagttgagggttgtatggtacaagtaacttgtggtacaactacacttaacggtctttggcttgatgacgtagtttactgtcca
agacatgtgatctgcacctctgaagacatgcttaaccctaattatgaagatttactcattcgtaagtctaatcataatttcttggtacaggctggtaat
gttcaactcagggttattggacattctatgcaaaattgtgtacttaagcttaaggttgatacagccaatcctaagacacctaagtataagtttgttcg
cattcaaccaggacagactttttcagtgttagcttgttacaatggttcaccatctggtgtttaccaatgtgctatgaggcccaatttcactattaagggt
tcattccttaatggttcatgtggtagtgttggttttaacatagattatgactgtgtctctttttgttacatgcaccatatggaattaccaactggagttcatg
ctggcacagacttagaaggtaacttttatggaccttttgttgacaggcaaacagcacaagcagctggtacggacacaactattacagttaatgttt
tagcttggttgtacgctgctgttataaatggagacaggtggtttctcaatcgatttaccacaactcttaatgactttaaccttgtggctatgaagtaca
attatgaacctctaacacaagaccatgttgacatactaggacctctttctgctcaaactggaattgccgttttagatatgtgtgcttcattaaaagaat
tactgcaaaatggtatgaatggacgtaccatattgggtagtgctttattagaagatgaatttacaccttttgatgttgttagacaatgctcaggtgtta
ctttccaaagtgcagtgaaaagaacaatcaagggtacacaccactggttgttactcacaattttgacttcacttttagttttagtccagagtactcaatggt
ctttgttcttttttttgtatgaaaatgcctttttaccttttgctatgggtattattgctatgtctgcttttgcaatgatgtttgtcaaacataagcatgcatttc
tctgtttgtttttgttaccttctcttgccactgtagcttattttaatatggtctatatgcctgctagttgggtgatgcgtattatgacatggttggatatggttg
atactagtttgtctggttttaagctaaaagactgtgttatgtatgcatcagctgtagtgttactaatccttatgacagcaagaactgtgtatgatgatggtg
ctaggagagtgtggacacttatgaatgtcttgacactcgtttataaagtttattatggtaatgctttagatcaagccatttccatgtgggctcttataatctc
tgttacttctaactactcaggtgtagttacaactgtcatgtttttggccagaggtattgtttttatgtgtgttgagtattgccctattttcttcataactggta
atacacttcagtgtataatgctagtttattgtttcttaggctatttttgtacttgttactttggcctcttttgtttactcaaccgctactttagactgactcttg
gtgtttatgattacttagtttctacacaggagtttagatatatgaattcacagggactactcccacccaagaatagcatagatgccttcaaactcaacatt
aaattgttgggtgttggtggcaaaccttgtatcaaagtagccactgtacagtctaaaatgtcagatgtaaagtgcacatcagtagtcttactctcag
ttttgcaacaactcagagtagaatcatcatctaaattgtgggctcaatgtgtccagttacacaatgacattctcttagctaaagatactactgaagc
ctttgaaaaaatggtttcactactttctgttttgctttccatgcagggtgctgtagacataaacaagctttgtgaagaaatgctggacaacagggca
accttacaagctatagcctcagagtttagttcccttccatcatatgcagcttttgctactgctcaagaagcttatgagcaggctgttgctaatggtgat
tctgaagttgttcttaaaaagttgaagaagtctttgaatgtggctaaatctgaatttgaccgtgatgcagccatgcaacgtaagttggaaaagatg
gctgatcaagctatgacccaaatgtataaacaggctagatctgaggacaagagggcaaaagttactagtgctatgcagacaatgcttttcact
atgcttagaaagttggataatgatgcactcaacaacattatcaacaatgcaagagatggttgtgttcccttgaacataatacctcttacaacagca
gccaaactaatggttgtcataccagactataacacatataaaaatacgtgtgatggtacaacatttacttatgcatcagcattgtgggaaatcca
acaggttgtagatgcagatagtaaaattgttcaacttagtgaaattagtatggacaattcacctaatttagcatggcctcttattgtaacagctttaag
ggccaattctgctgtcaaattacagaataatgagcttagtcctgttgcactacgacagatgtcttgtgctgccggtactacacaaactgcttgcact
gatgacaatgcgttagcttactacaacacaacaaagggaggtaggtttgtacttgcactgttatccgatttacaggatttgaaatgggctagattc
cctaagagtgatggaactggtactatctatacagaactggaaccaccttgtaggtttgttacagacacacctaaaggtcctaaagtgaagtattt
atactttattaaaggattaaacaacctaaatagaggtatggtacttggtagtttagctgccacagtacgtctacaagctggtaatgcaacagaagt
gcctgccaattcaactgtattatctttctgtgcttttgctgtagatgctgctaaagcttacaaagattatctagctagtgggggacaaccaatcactaa
ttgtgttaagatgttgtgtacacacactggtactggtcaggcaataacagttacaccggaagccaatatggatcaagaatcctttggtggtgcatc
gtgttgtctgtactgccgttgccacatagatcatccaaatcctaaaggattttgtgacttaaaaggtaagtatgtacaaatacctacaacttgtgcta
atgaccctgtgggttttacacttaaaaacacagtctgtaccgtctgcggtatgtggaaaggttatggctgtagttgtgatcaactccgcgaacccat
gcttcagtcagctgatgcacaatcgtttttaaacgggtttgcggtgtaagtgcagcccgtcttacaccgtgcggcacaggcactagtactgatgtc
gtatacagggcttttgacatctacaatgataaagtagctggttttgctaaattcctaaaaactaattgttgtcgcttccaagaaaaggacgaagatg
acaatttaattgattcttactttgtagttaagagacacactttctctaactaccaacatgaagaaacaatttataatttacttaaggattgtccagctgtt
gctaaacatgacttctttaagtttagaatagacggtgacatggtaccacatatatcacgtcaacgtcttactaaatacacaatggcagacctcgtc
tatgctttaaggcattttgatgaaggtaattgtgacacattaaaagaaatacttgtcacatacaattgttgtgatgatgattatttcaataaaaaggac
tggtatgattttgtagaaaacccagatatattacgcgtatacgccaacttaggtgaacgtgtacgccaagctttgttaaaaacagtacaattctgtg
atgccatgcgaaatgctggtattgttggtgtactgacattagataatcaagatctcaatggtaactggtatgatttcggtgatttcatacaaaccacg
ccaggtagtggagttcctgttgtagattcttattattcattgttaatgcctatattaaccttgaccagggctttaactgcagagtcacatgttgacactga
cttaacaaagccttacattaagtgggatttgttaaaatatgacttcacggaagagaggttaaaactctttgaccgttattttaaatattgggatcaga
cataccacccaaattgtgttaactgtttggatgacagatgcattctgcattgtgcaaactttaatgttttattctctacagtgttcccacctacaagttttg
gaccactagtgagaaaaatatttgttgatggtgttccatttgtagtttcaactggataccacttcagagagctaggtgttgtacataatcaggatgta
aacttacatagctccagacttagttttaaggaattacttgtgtatgctgctgaccctgctatgcacgctgcttctggtaatctattactagataaacgc
actacgtgcttttcagtagctgcacttactaacaatgttgcttttcaaactgtcaaacccggtaattttaacaaagacttctatgactttgctgtgtctaa
gggtttctttaaggaaggaagttctgttgaattaaaacacttcttctttgctcaggatggtaatgctgctatcagcgattatgactactatcgttataatc
taccaacaatgtgtgatatcagacaactactatttgtagttgaagttgttgataagtactttgattgttacgatggtggctgtattaatgctaaccaagt
catcgtcaacaacctagacaaatcagctggttttccatttaataaatggggtaaggctagactttattatgattcaatgagttatgaggatcaagat
gcacttttcgcatatacaaaacgtaatgtcatccctactataactcaaatgaatcttaagtatgccattagtgcaaagaatagagctcgcaccgta
gctggtgtctctatctgtagtactatgaccaatagacagtttcatcaaaaattattgaaatcaatagccgccactagaggagctactgtagtaattg
gaacaagcaaattctatggtggttggcacaacatgttaaaaactgtttatagtgatgtagaaaaccctcaccttatgggttgggattatcctaaatg
tgatagagccatgcctaacatgcttagaattatggcctcacttgttcttgctcgcaaacatacaacgtgttgtagcttgtcacaccgtttctatagatt
agctaatgagtgtgctcaagtattgagtgaaatggtcatgtgtggcggttcactatatgttaaaccaggtggaacctcatcaggagatgccacaa
ctgcttatgctaatagtgtttttaacatttgtcaagctgtcacggccaatgttaatgcacttttatctactgatggtaacaaaattgccgataagtatgtc
cgcaatttacaacacagactttatgagtgtctctatagaaatagagatgttgacacagactttgtgaatgagttttacgcatatttgcgtaaacatttc
tcaatgatgatactctctgacgatgctgttgtgtgtttcaatagcacttatgcatctcaaggtctagtggctagcataaagaactttaagtcagttcttt
attatcaaaacaatgtttttatgtctgaagcaaaatgttggactgagactgaccttactaaaggacctcatgaattttgctctcaacatacaatgcta
gttaaacagggtgatgattatgtgtaccttccttacccagatccatcaagaatcctaggggccggctgttttgtagatgatatcgtaaaaacagat
ggtacacttatgattgaacggttcgtgtctttagctatagatgcttacccacttactaaacatcctaatcaggagtatgctgatgtctttcatttgtactta
caatacataagaaagctacatgatgagttaacaggacacatgttagacatgtattctgttatgcttactaatgataacacttcaaggtattgggaa
cctgagttttatgaggctatgtacacaccgcatacagtcttacaggctgttggggcttgtgttctttgcaattcacagacttcattaagatgtggtgctt
gcatacgtagaccattcttatgttgtaaatgctgttacgaccatgtcatatcaacatcacataaattagtcttgtctgttaatccgtatgtttgcaatgct
ccaggttgtgatgtcacagatgtgactcaactttacttaggaggtatgagctattattgtaaatcacataaaccacccattagttttccattgtgtgcta
atggacaagtttttggtttatataaaaatacatgtgttggtagcgataatgttactgactttaatgcaattgcaacatgtgactggacaaatgctggtg
attacattttagctaacacctgtactgaaagactcaagctttttgcagcagaaacgctcaaagctactgaggagacatttaaactgtcttatggtatt
gctactgtacgtgaagtgctgtctgacagagaattacatctttcatgggaagttggtaaacctagaccaccacttaaccgaaattatgtctttactg
gttatcgtgtaactaaaaacagtaaagtacaaataggagagtacacctttgaaaaaggtgactatggtgatgctgttgtttaccgaggtacaac
aacttacaaattaaatgttggtgattattttgtgctgacatcacatacagtaatgccattaagtgcacctacactagtgccacaagagcactatgtta
gaattactggcttatacccaacactcaatatctcagatgagttttctagcaatgttgcaaattatcaaaaggttggtatgcaaaagtattctacactc
cagggaccacctggtactggtaagagtcattttgctattggcctagctctctactacccttctgctcgcatagtgtatacagcttgctctcatgccgct
gttgatgcactatgtgagaaggcattaaaatatttgcctatagataaatgtagtagaattatacctgcacgtgctcgtgtagagtgttttgataaattc
aaagtgaattcaacattagaacagtatgtcttttgtactgtaaatgcattgcctgagactacagcagatatagttgtctttgatgaaatttcaatggcc
acaaattatgatttgagtgttgtcaatgccagattacgtgctaagcactatgtgtacattggcgaccctgctcaattacctgcaccacgcacattgct
aactaagggcacactagaaccagaatatttcaattcagtgtgtagacttatgaaaactataggtccagacatgttcctcggaacttgtcggcgttg
tcctgctgaaattgttgacactgtgagtgctttggtttatgataataagcttaaagcacataaagacaaatcagctcaatgctttaaaatgttttataa
gggtgttatcacgcatgatgtttcatctgcaattaacaggccacaaataggcgtggtaagagaattccttacacgtaaccctgcttggagaaaag
ctgtctttatttcaccttataattcacagaatgctgtagcctcaaagattttgggactaccaactcaaactgttgattcatcacagggctcagaatatg
actatgtcatattcactcaaaccactgaaacagctcactcttgtaatgtaaacagatttaatgttgctattaccagagcaaaagtaggcatactttg
cataatgtctgatagagacttatatgacaagttgcaatttacaagtcttgaaattccacgtaggaatgtggcaactttacaagctgaaaatgtaac
aggactctttaaagattgtagtaaggtaatcactgggttacatcctacacaggcacctacacacctcagtgttgacactaaattcaaaactgaag
gtttatgtgttgacatacctggcatacctaaggacatgacctatagaagactcatctctatgatgggttttaaaatgaattatcaagttaatggttacc
ctaacatgtttatcacccgcgaagaagctataagacatgtacgtgcatggattggcttcgatgtcgaggggtgtcatgctactagagaagctgtt
ggtaccaatttacctttacagctaggtttttctacaggtgttaacctagttgctgtacctacaggttatgttgatacacctaataatacagatttttccag
agttagtgctaaaccaccgcctggagatcaatttaaacacctcataccacttatgtacaaaggacttccttggaatgtagtgcgtataaagattgt
acaaatgttaagtgacacacttaaaaatctctctgacagagtcgtatttgtcttatgggcacatggctttgagttgacatctatgaagtattttgtgaa
aataggacctgagcgcacctgttgtctatgtgatagacgtgccacatgcttttccactgcttcagacacttatgcctgttggcatcattctattggattt
gattacgtctataatccgtttatgattgatgttcaacaatggggttttacaggtaacctacaaagcaaccatgatctgtattgtcaagtccatggtaat
gcacatgtagctagttgtgatgcaatcatgactaggtgtctagctgtccacgagtgctttgttaagcgtgttgactggactattgaatatcctataatt
ggtgatgaactgaagattaatgcggcttgtagaaaggttcaacacatggttgttaaagctgcattattagcagacaaattcccagttcttcacgac
attggtaaccctaaagctattaagtgtgtacctcaagctgatgtagaatggaagttctatgatgcacagccttgtagtgacaaagcttataaaata
gaagaattattctattcttatgccacacattctgacaaattcacagatggtgtatgcctattttggaattgcaatgtcgatagatatcctgctaattccat
tgtttgtagatttgacactagagtgctatctaaccttaacttgcctggttgtgatggtggcagtttgtatgtaaataaacatgcattccacacaccagct
tttgataaaagtgcttttgttaatttaaaacaattaccatttttctattactctgacagtccatgtgagtctcatggaaaacaagtagtgtcagatataga
ttatgtaccactaaagtctgctacgtgtataacacgttgcaatttaggtggtgctgtctgtagacatcatgctaatgagtacagattgtatctcgatgc
ttataacatgatgatctcagctggctttagcttgtgggtttacaaacaatttgatacttataacctctggaacacttttacaagacttcagagtttagaa
aatgtggcttttaatgttgtaaataagggacactttgatggacaacagggtgaagtaccagtttctatcattaataacactgtttacacaaaagttg
atggtgttgatgtagaattgtttgaaaataaaacaacattacctgttaatgtagcatttgagctttgggctaagcgcaacattaaaccagtaccaga
ggtgaaaatactcaataatttgggtgtggacattgctgctaatactgtgatctgggactacaaaagagatgctccagcacatatatctactattggt
gtttgttctatgactgacatagccaagaaaccaactgaaacgatttgtgcaccactcactgtcttttttgatggtagagttgatggtcaagtagactt
atttagaaatgcccgtaatggtgttcttattacagaaggtagtgttaaaggtttacaaccatctgtaggtcccaaacaagctagtcttaatggagtc
acattaattggagaagccgtaaaaacacagttcaattattataagaaagttgatggtgttgtccaacaattacctgaaacttactttactcagagta
gaaatttacaagaatttaaacccaggagtcaaatggaaattgatttcttagaattagctatggatgaattcattgaacggtataaattagaaggct
atgccttcgaacatatcgtttatggagattttagtcatagtcagttaggtggtttacatctactgattggactagctaaacgttttaaggaatcacctttt
gaattagaagattttattcctatggacagtacagttaaaaactatttcataacagatgcgcaaacaggttcatctaagtgtgtgtgttctgttattgatt
tattacttgatgattttgttgaaataataaaatcccaagatttatctgtagtttctaaggttgtcaaagtgactattgactatacagaaatttcatttatgct
ttggtgtaaagatggccatgtagaaacattttacccaaaattacaatctagtcaagcgtggcaaccgggtgttgctatgcctaatctttacaaaat
gcaaagaatgctattagaaaagtgtgaccttcaaaattatggtgatagtgcaacattacctaaaggcataatgatgaatgtcgcaaaatatactc
aactgtgtcaatatttaaacacattaacattagctgtaccctataatatgagagttatacattttggtgctggttctgataaaggagttgcaccaggta
cagctgttttaagacagtggttgcctacgggtacgctgcttgtcgattcagatcttaatgactttgtctctgatgcagattcaactttgattggtgattgt
gcaactgtacatacagctaataaatgggatctcattattagtgatatgtacgaccctaagactaaaaatgttacaaaagaaaatgactctaaag
agggttttttcacttacatttgtgggtttatacaacaaaagctagctcttggaggttccgtggctataaagataacagaacattcttggaatgctgatc
tttataagctcatgggacacttcgcatggtggacagcctttgttactaatgtgaatgcgtcatcatctgaagcatttttaattggatgtaattatcttggc
aaaccacgcgaacaaatagatggttatgtcatgcatgcaaattacatattttggaggaatacaaatccaattcagttgtcttcctattctttatttgac
atgagtaaatttccccttaaattaaggggtactgctgttatgtctttaaaagaaggtcaaatcaatgatatgattttatctcttcttagtaaaggtagac
ttataattagagaaaacaacagagttgttatttctagtgatgttcttgttaacaactaaGTTTAAACctggcgcgccagaataaacgaacgcc
accatggtgagcaagggcgaggagctgttcaccggggtggtgcccatcctggtcgagctggacggcgacgtaaacggccacaagttcagc
gtgtccggcgagggcgagggcgatgccacctacggcaagctgaccctgaagttcatctgcaccaccggcaagctgcccgtgccctggccc
accctcgtgaccaccctgacctacggcgtgcagtgcttcagccgctaccccgaccacatgaagcagcacgacttcttcaagtccgccatgccc
gaaggctacgtccaggagcgcaccatcttcttcaaggacgacggcaactacaagacccgcgccgaggtgaagttcgagggcgacaccct
ggtgaaccgcatcgagctgaagggcatcgacttcaaggaggacggcaacatcctggggcacaagctggagtacaactacaacagccaca
acgtctatatcatggccgacaagcagaagaacggcatcaaggtgaacttcaagatccgccacaacatcgaggacggcagcgtgcagctcg
ccgaccactaccagcagaacacccccatcggcgacggccccgtgctgctgcccgacaaccactacctgagcacccagtccgccctgagc
aaagaccccaacgagaagcgcgatcacatggtcctgctggagttcgtgaccgccgccgggatcactctcggcatggacgagctgtacaagt

| Feature list | | | | |
|---|---|---|---|---|
| ATGC | 1 . . . 4 | 4 | == | homology |
| source | 5 . . . 10 | 6 | == | source |
| XbaI restriction site | 5 . . . 10 | 6 | == | misc_feature |
| source | 11 . . . 12 | 2 | == | source |
| CT dinucleotide | 11 . . . 12 | 2 | == | misc_feature |
| source (part) | 13 . . . 20 | 8 | == | source |
| source (part) | 13 . . . 20 | 8 | == | source |
| SbfI restriction site (part) | 13 . . . 20 | 8 | == | misc_feature |
| source | 24 . . . 32 | 9 | == | source |
| RNA | 24 . . . 32 | 9 | == | misc_feature |
| STOP | 24 . . . 26 | 3 | == | misc_feature |
| ORF10 (part) | 33 . . . 149 | 117 | => | gene |
| ORF10 (part) | 33 . . . 149 | 117 | => | CDS |
| stem loop (part) | 84 . . . 119 | 36 | == | stem_loop |
| stem loop (part) | 104 . . . 132 | 29 | == | stem_loop |
| 3′ UTR (part) | 150 . . . 378 | 229 | == | 3′UTR |
| stem loop (part) | 203 . . . 243 | 41 | == | stem_loop |
| source | 379 . . . 386 | 8 | == | source |
| NotI restriction site | 379 . . . 386 | 8 | == | misc_feature |
| From cov2_frag_1_6 | 379 . . . 386 | 8 | == | misc_feature |
| HDV ribozyme | 387 . . . 454 | 68 | => | ncRNA |
| bGH poly(A) signal | 455 . . . 679 | 225 | == | polyA_signal |
| AmpR promoter (part) | 680 . . . 784 | 105 | == | promoter |
| CTAA | 1220 . . . 1223 | 4 | == | homology |
| G −> C (remove BsaI) (part) | 1501 . . . 1501 | 1 | == | misc_feature |
| origin (part) | 1816 . . . 2404 | 589 | == | rep_origin |
| pBR322 origin, forward primer (part) | 2305 . . . 2324 | 20 | => | primer_bind |
| source | 2425 . . . 2432 | 8 | == | source |
| AsiSI restriction site | 2425 . . . 2432 | 8 | == | misc_feature |
| CMV enhancer | 2570 . . . 2949 | 380 | == | enhancer |
| CMV promoter | 2950 . . . 3153 | 204 | => | promoter |
| chimeric intron | 3289 . . . 3421 | 133 | == | intron |
| T7 promoter | 3466 . . . 3485 | 20 | == | misc_feature |
| promoter (part) | 3467 . . . 3485 | 19 | == | promoter |
| G > A | 3483 . . . 3483 | 1 | == | misc_feature |
| source | 3486 . . . 3493 | 8 | == | source |
| source (part) | 3486 . . . 3493 | 8 | == | source |
| source (part) | 3486 . . . 3493 | 8 | == | source |
| source (part) | 3486 . . . 3493 | 8 | == | source |
| FseI restriction site (part) | 3486 . . . 3493 | 8 | == | misc_feature |
| 5′ UTR (part) | 3494 . . . 3758 | 265 | == | 5′UTR |
| left_flank | 3494 . . . 3494 | 1 | == | misc_feature |
| Kozak sequence | 3759 . . . 3764 | 6 | == | regulatory |
| mature peptide (part) | 3765 . . . 4304 | 540 | == | mat_peptide |
| source | 4305 . . . 4316 | 12 | == | source |
| Finalised sequence | 4305 . . . 4316 | 12 | == | misc_feature |
| mature peptide (part) | 4317 . . . 5234 | 918 | == | mat_peptide |
| TTCT | 5453 . . . 5456 | 4 | == | homology |
| mature peptide (part) | 6105 . . . 6353 | 249 | == | mat_peptide |
| mature peptide (part) | 6354 . . . 6947 | 594 | == | mat_peptide |
| mature peptide (part) | 6948 . . . 7286 | 339 | == | mat_peptide |
| mature peptide (part) | 7287 . . . 7703 | 417 | == | mat_peptide |
| mature peptide (part) | 7704 . . . 7742 | 39 | == | mat_peptide |
| mature peptide (part) | 7704 . . . 7730 | 27 | == | mat_peptide |
| stem loop (part) | 7738 . . . 7765 | 28 | == | stem_loop |
| stem loop (part) | 7750 . . . 7804 | 55 | == | stem_loop |
| T −> C remove XbaI | 8794 . . . 8794 | 1 | == | misc_feature |
| ATGC | 9276 . . . 9279 | 4 | == | homology |
| mature peptide (part) | 10,499 . . . 12,301 | 1803 | == | mat_peptide |
| ACCT | 13,324 . . . 13,327 | 4 | == | homology |
| mature peptide (part) | 13,883 . . . 14,920 | 1038 | == | mat_peptide |
| mature peptide (part) | 14,921 . . . 15,814 | 894 | == | mat_peptide |
| source | 15,826 . . . 15,827 | 2 | == | source |

CMV + T7_COV-2_GFP_HBA1
(SEQ ID NO: 19)
atgctctagactcctgcaggGTTtaaacgaacatgggctatataaacgttttcgcttttccgtttacgatatatagtctactcttgtgcagaatgaat
tctcgtaactacatagcacaagtagatgtagttaactttaatctcacatagcaatctttaatcagtgtgtaacattagggaggacttgaaagagcc
accacattttcaccgaggccacgcggagtacgatcgagtgtacagtgaacaatgctagggagagctgcctatatggaagagccctaatgtgt
aaaattaattttagtagtgctatccccatgtgattttaatagcttcttaggagaatgacaaaaaaaaaaaaaaaaaaaaaaaaaaaaaaaaa
gcggccgcGGCCGGCATGGTCCCAGCCTCCTCGCTGGCGCCGGCTGGGCAACATGCTTCGGCATG
GCGAATGGGACaataaagtctgagtgggcggcacgcggaacccctatttgtttatttttctaaatacattcaaatatgtatccgctcatgag
acaataaccctgataaatgcttcaataatattgaaaaaggaagagtatgagtattcaacatttccgtgtcgcccttattcccttttttgcggcattttgc
cttcctgtttttgctcacccagaaacgctggtgaaagtaaaagatgctgaagatcagttgggtgcacgagtgggttacatcgaactggatctcaa
cagcggtaagatccttgagagttttcgccccgaagaacgttttccaatgatgagcacttttaaagttctgctatgtggcgcggtattatcccgtgttg
acgccgggcaagagcaactcggtcgccgcatacactattctcagaatgacttggttgagtactcaccagtcacagaaaagcatcttacggatg
gcatgacagtaagagaattatgcagtgctgccataaccatgagtgataacactgcggccaacttacttctgacaacgatcggaggaccgaag
gagctaaccgcttttttgcacaacatgggggatcatgtaactcgccttgatcgttgggaaccggagctgaatgaagccataccaaacgacgag
cgtgacaccacgatgcctgtagcaatggcaacaacgttgcgcaaactattaactggcgaactacttactctagcttcccggcaacaattaatag
actggatggaggcggataaagttgcaggaccacttctgcgctcggcccttccggctggctggtttattgctgataaatctggagccggtgagcgt
ggctctcgcggtatcattgcagcactggggccagatggtaagccctcccgtatcgtagttatctacacgacggggagtcaggcaactatggatg
aacgaaatagacagatcgctgagataggtgcctcactgattaagcattggtaactgtcagaccaagtttactcatatatactttagattgatttaaa
acttcatttttaatttaaaaggatctaggtgaagatcctttttgataatctcatgaccaaaatcccttaacgtgagttttcgttccactgagcgtcagac
cccgtagaaaagatcaaaggatcttcttgagatcctttttttctgcgcgtaatctgctgcttgcaaacaaaaaaaccaccgctaccagcggtggttt
gtttgccggatcaagagctaccaactctttttccgaaggtaactggcttcagcagagcgcagataccaaatactgtccttctagtgtagccgtagtt
aggccaccacttcaagaactctgtagcaccgcctacatacctcgctctgctaatcctgttaccagtggctgctgccagtggcgataagtcgtgtct
taccgggttggactcaagacgatagttaccggataaggcgcagcggtcgggctgaacggggggttcgtgcacacagcccagcttggagcg
aacgacctacaccgaactgagatacctacagcgtgagctatgagaaagcgccacgcttcccgaagggagaaaggcggacaggtatccgg
taagcggcagggtcggaacaggagagcgcacgagggagcttccagggggaaacgcctggtatctttatagtcctgtcgggtttcgccacctc
tgacttgagcgtcgatttttgtgatgctcgtcaggggggcggagcctatggaaaaacgccagcaacgcgagctcgcgatcgctcaatattggc
cattagccatattattcattggttatatagcataaatcaatattggctattggccattgcatacgttgtatctatatcataatatgtacatttatattggctc
atgtccaatatgaccgccatgttggcattgattattgactagttattaatagtaatcaattacggggtcattagttcatagcccatatatggagttccg
cgttacataacttacggtaaatggcccgcctggctgaccgcccaacgacccccgcccattgacgtcaataatgacgtatgttcccatagtaacg
ccaatagggactttccattgacgtcaatgggtggagtatttacggtaaactgcccacttggcagtacatcaagtgtatcatatgccaagtccgccc
cctattgacgtcaatgacggtaaatggcccgcctggcattatgcccagtacatgaccttacgggactttcctacttggcagtacatctacgtattag
tcatcgctattaccatggtgatgcggttttggcagtacaccaatgggcgtggatagcggtttgactcacggggatttccaagtctccaccccattga
cgtcaatgggagtttgttttggcaccaaaatcaacgggactttccaaaatgtcgtaataaccccgccccgttgacgcaaatgggcggtaggcgt
gtacggtgggaggtctatataagcagagctcgtttagtgaaccgtcagatcactagaagctttattgcggtagtttatcacagttaaattgctaacg
cagtcagtgcttctgacacaacagtctcgaacttaagctgcagaagttggtcgtgaggcactgggcaggtaagtatcaaggttacaagacagg
tttaaggagaccaatagaaactgggcttgtcgagacagagaagactcttgcgtttctgataggcacctattggtcttactgacatccactttgccttt
ctctccacagGTGTCCACTCCCAGTTCAATTACAGCTCTTAAGGCTAGAGTACTtaatacgactcactataaggg
gccggccattaaaggtttataccttcccaggtaacaaaccaaccaactttcgatctcttgtagatctgttctctaaacgaactttaaaatctgtgtgg
ctgtcactcggctgcatgcttagtgcactcacgcagtataattaataactaattactgtcgttgacaggacacgagtaactcgtctatcttctgcagg
ctgcttacggtttcgtccgtgttgcagccgatcatcagcacatctaggtttcgtccgggtgtgaccgaaaggtaagactcttctggtccccacaga
ctcagagagaaGCCACCatggagagccttgtccctggtttcaacgagaaaacacacgtccaactcagtttgcctgttttacaggttcgcgac
gtgctcgtacgtggctttggagactccgtggaggaggtcttatcagaggcacgtcaacatcttaaagatggcacttgtggcttagtagaagttga
aaaaggcgttttgcctcaacttgaacagccctatgtgttcatcaaacgttcggatgctcgaactgcacctcatggtcatgttatggttgagctggta
gcagaactcgaaggcattcagtacggtcgtagtggtgagacacttggtgtccttgtccctcatgtgggcgaaataccagtggcttaccgcaagg
ttcttcttcgtaagaacggtaataaaggagctggtggccatagttacggcgccgatctaaagtcatttgacttaggcgacgagcttggcactgatc
cttatgaagattttcaagaaaactggaacactaaacatagcagtggtgttacccgtgaactcatgcgtgagcttaacggaggggctgttttgcag
agtggttttagaaaaatggcattcccatctggtaaagttgagggttgtatggtacaagtaacttgtggtacaactacacttaacggtctttggcttgat
gacgtagtttactgtccaagacatgtgatctgcacctctgaagacatgcttaaccctaattatgaagatttactcattcgtaagtctaatcataatttct
tggtacaggctggtaatgttcaactcagggttattggacattctatgcaaaattgtgtacttaagcttaaggttgatacagccaatcctaagacacc
taagtataagtttgttcgcattcaaccaggacagactttttcagtgttagcttgttacaatggttcaccatctggtgtttaccaatgtgctatgaggccc
aatttcactattaagggttcattccttaatggttcatgtggtagtgttggttttaacatagattatgactgtgtctctttttgttacatgcaccatatggaat
taccaactggagttcatgctggcacagacttagaaggtaacttttatggaccttttgttgacaggcaaacagcacaagcagctggtacggacacaacta
ttacagttaatgttttagcttggttgtacgctgctgttataaatggagacaggtggtttctcaatcgatttaccacaactcttaatgactttaacctt
gtggctatgaagtacaattatgaacctctaacacaagaccatgttgacatactaggacctctttctgctcaaactggaattgccgttttagatatgtg
tgcttcattaaaagaattactgcaaaatggtatgaatggacgtaccatattgggtagtgctttattagaagatgaatttacaccttttgatgttgttaga
caatgctcaggtgttactttccaaagtgcagtgaaaagaacaatcaagggtacacaccactggttgttactcacaattttgacttcacttttagttttagtcc
agagtactcaatggtctttgttcttttttttgtatgaaaatgcctttttaccttttgctatgggtattattgctatgtctgcttttgcaatgatgtttgtcaaa
cataagcatgcatttctctgtttgtttttgttaccttctcttgccactgtagcttattttaatatggtctatatgcctgctagttgggtgatgcgtattatgac
atggttggatatggttgatactagtttgtctggttttaagctaaaagactgtgttatgtatgcatcagctgtagtgttactaatccttatgacagcaagaac
tgtgtatgatgatggtgctaggagagtgtggacacttatgaatgtcttgacactcgtttataaagtttattatggtaatgctttagatcaagccatttccatgt
gggctcttataatctctgttacttctaactactcaggtgtagttacaactgtcatgtttttggccagaggtattgtttttatgtgtgttgagtattgccct
attttcttcataactggtaatacacttcagtgtataatgctagtttattgtttcttaggctatttttgtacttgttactttggcctcttttgtttactcaacc
gctactttagactgactcttggtgtttatgattacttagtttctacacaggagtttagatatatgaattcacagggactactcccacccaagaatagcatagat
gccttcaaactcaacattaaattgttgggtgttggtggcaaaccttgtatcaaagtagccactgtacagtctaaaatgtcagatgtaaagtgcaca
tcagtagtcttactctcagttttgcaacaactcagagtagaatcatcatctaaattgtgggctcaatgtgtccagttacacaatgacattctcttagct
aaagatactactgaagcctttgaaaaaatggtttcactactttctgttttgctttccatgcagggtgctgtagacataaacaagctttgtgaagaaat
gctggacaacagggcaaccttacaagctatagcctcagagtttagttcccttccatcatatgcagcttttgctactgctcaagaagcttatgagca
ggctgttgctaatggtgattctgaagttgttcttaaaaagttgaagaagtctttgaatgtggctaaatctgaatttgaccgtgatgcagccatgcaac
gtaagttggaaaagatggctgatcaagctatgacccaaatgtataaacaggctagatctgaggacaagagggcaaaagttactagtgctatg
cagacaatgcttttcactatgcttagaaagttggataatgatgcactcaacaacattatcaacaatgcaagagatggttgtgttcccttgaacata
atacctcttacaacagcagccaaactaatggttgtcataccagactataacacatataaaaatacgtgtgatggtacaacatttacttatgcatca
gcattgtgggaaatccaacaggttgtagatgcagatagtaaaattgttcaacttagtgaaattagtatggacaattcacctaatttagcatggcctc
ttattgtaacagctttaagggccaattctgctgtcaaattacagaataatgagcttagtcctgttgcactacgacagatgtcttgtgctgccggtacta
cacaaactgcttgcactgatgacaatgcgttagcttactacaacacaacaaagggaggtaggtttgtacttgcactgttatccgatttacaggattt
gaaatgggctagattccctaagagtgatggaactggtactatctatacagaactggaaccaccttgtaggtttgttacagacacacctaaaggtc
ctaaagtgaagtatttatactttattaaaggattaaacaacctaaatagaggtatggtacttggtagtttagctgccacagtacgtctacaagctggt
aatgcaacagaagtgcctgccaattcaactgtattatctttctgtgcttttgctgtagatgctgctaaagcttacaaagattatctagctagtggggg
acaaccaatcactaattgtgttaagatgttgtgtacacacactggtactggtcaggcaataacagttacaccggaagccaatatggatcaagaa
tcctttggtggtgcatcgtgttgtctgtactgccgttgccacatagatcatccaaatcctaaaggattttgtgacttaaaaggtaagtatgtacaaata
cctacaacttgtgctaatgaccctgtgggttttacacttaaaaacacagtctgtaccgtctgcggtatgtggaaaggttatggctgtagttgtgatca
actccgcgaacccatgcttcagtcagctgatgcacaatcgtttttaaacgggtttgcggtgtaagtgcagcccgtcttacaccgtgcggcacagg
cactagtactgatgtcgtatacagggcttttgacatctacaatgataaagtagctggttttgctaaattcctaaaaactaattgttgtcgcttccaaga
aaaggacgaagatgacaatttaattgattcttactttgtagttaagagacacactttctctaactaccaacatgaagaaacaatttataatttactta
aggattgtccagctgttgctaaacatgacttctttaagtttagaatagacggtgacatggtaccacatatatcacgtcaacgtcttactaaatacac
aatggcagacctcgtctatgctttaaggcattttgatgaaggtaattgtgacacattaaaagaaatacttgtcacatacaattgttgtgatgatgatt
atttcaataaaaaggactggtatgattttgtagaaaacccagatatattacgcgtatacgccaacttaggtgaacgtgtacgccaagctttgttaa
aaacagtacaattctgtgatgccatgcgaaatgctggtattgttggtgtactgacattagataatcaagatctcaatggtaactggtatgatttcggt
gatttcatacaaaccacgccaggtagtggagttcctgttgtagattcttattattcattgttaatgcctatattaaccttgaccagggctttaactgcag
agtcacatgttgacactgacttaacaaagccttacattaagtgggatttgttaaaatatgacttcacggaagagaggttaaaactctttgaccgtta
ttttaaatattgggatcagacataccacccaaattgtgttaactgtttggatgacagatgcattctgcattgtgcaaactttaatgttttattctctacagt
gttcccacctacaagttttggaccactagtgagaaaaatatttgttgatggtgttccatttgtagtttcaactggataccacttcagagagctaggtgt
tgtacataatcaggatgtaaacttacatagctccagacttagttttaaggaattacttgtgtatgctgctgaccctgctatgcacgctgcttctggtaat
ctattactagataaacgcactacgtgcttttcagtagctgcacttactaacaatgttgcttttcaaactgtcaaacccggtaattttaacaaagacttc
tatgactttgctgtgtctaagggtttctttaaggaaggaagttctgttgaattaaaacacttcttctttgctcaggatggtaatgctgctatcagcgattat
gactactatcgttataatctaccaacaatgtgtgatatcagacaactactatttgtagttgaagttgttgataagtactttgattgttacgatggtggctg
tattaatgctaaccaagtcatcgtcaacaacctagacaaatcagctggttttccatttaataaatggggtaaggctagactttattatgattcaatga
gttatgaggatcaagatgcacttttcgcatatacaaaacgtaatgtcatccctactataactcaaatgaatcttaagtatgccattagtgcaaaga
atagagctcgcaccgtagctggtgtctctatctgtagtactatgaccaatagacagtttcatcaaaaattattgaaatcaatagccgccactagag
gagctactgtagtaattggaacaagcaaattctatggtggttggcacaacatgttaaaaactgtttatagtgatgtagaaaaccctcaccttatgg
gttgggattatcctaaatgtgatagagccatgcctaacatgcttagaattatggcctcacttgttcttgctcgcaaacatacaacgtgttgtagcttgt
cacaccgtttctatagattagctaatgagtgtgctcaagtattgagtgaaatggtcatgtgtggcggttcactatatgttaaaccaggtggaacctc
atcaggagatgccacaactgcttatgctaatagtgtttttaacatttgtcaagctgtcacggccaatgttaatgcacttttatctactgatggtaacaa
aattgccgataagtatgtccgcaatttacaacacagactttatgagtgtctctatagaaatagagatgttgacacagactttgtgaatgagttttacg
catatttgcgtaaacatttctcaatgatgatactctctgacgatgctgttgtgtgtttcaatagcacttatgcatctcaaggtctagtggctagcataaa
gaactttaagtcagttctttattatcaaaacaatgtttttatgtctgaagcaaaatgttggactgagactgaccttactaaaggacctcatgaattttgc
tctcaacatacaatgctagttaaacagggtgatgattatgtgtaccttccttacccagatccatcaagaatcctaggggccggctgttttgtagatg
atatcgtaaaaacagatggtacacttatgattgaacggttcgtgtctttagctatagatgcttacccacttactaaacatcctaatcaggagtatgct
gatgtctttcatttgtacttacaatacataagaaagctacatgatgagttaacaggacacatgttagacatgtattctgttatgcttactaatgataac
acttcaaggtattgggaacctgagttttatgaggctatgtacacaccgcatacagtcttacaggctgttggggcttgtgttctttgcaattcacagact
tcattaagatgtggtgcttgcatacgtagaccattcttatgttgtaaatgctgttacgaccatgtcatatcaacatcacataaattagtcttgtctgttaa
tccgtatgtttgcaatgctccaggttgtgatgtcacagatgtgactcaactttacttaggaggtatgagctattattgtaaatcacataaaccacccat
tagttttccattgtgtgctaatggacaagtttttggtttatataaaaatacatgtgttggtagcgataatgttactgactttaatgcaattgcaacatgtga
ctggacaaatgctggtgattacattttagctaacacctgtactgaaagactcaagctttttgcagcagaaacgctcaaagctactgaggagacat
ttaaactgtcttatggtattgctactgtacgtgaagtgctgtctgacagagaattacatctttcatgggaagttggtaaacctagaccaccacttaac
cgaaattatgtctttactggttatcgtgtaactaaaaacagtaaagtacaaataggagagtacacctttgaaaaaggtgactatggtgatgctgtt
gtttaccgaggtacaacaacttacaaattaaatgttggtgattattttgtgctgacatcacatacagtaatgccattaagtgcacctacactagtgcc
acaagagcactatgttagaattactggcttatacccaacactcaatatctcagatgagttttctagcaatgttgcaaattatcaaaaggttggtatg
caaaagtattctacactccagggaccacctggtactggtaagagtcattttgctattggcctagctctctactacccttctgctcgcatagtgtatac
agcttgctctcatgccgctgttgatgcactatgtgagaaggcattaaaatatttgcctatagataaatgtagtagaattatacctgcacgtgctcgtg
tagagtgttttgataaattcaaagtgaattcaacattagaacagtatgtcttttgtactgtaaatgcattgcctgagactacagcagatatagttgtctt
tgatgaaatttcaatggccacaaattatgatttgagtgttgtcaatgccagattacgtgctaagcactatgtgtacattggcgaccctgctcaattac
ctgcaccacgcacattgctaactaagggcacactagaaccagaatatttcaattcagtgtgtagacttatgaaaactataggtccagacatgttc
ctcggaacttgtcggcgttgtcctgctgaaattgttgacactgtgagtgctttggtttatgataataagcttaaagcacataaagacaaatcagctc
aatgctttaaaatgttttataagggtgttatcacgcatgatgtttcatctgcaattaacaggccacaaataggcgtggtaagagaattccttacacgt
aaccctgcttggagaaaagctgtctttatttcaccttataattcacagaatgctgtagcctcaaagattttgggactaccaactcaaactgttgattc
atcacagggctcagaatatgactatgtcatattcactcaaaccactgaaacagctcactcttgtaatgtaaacagatttaatgttgctattaccaga
gcaaaagtaggcatactttgcataatgtctgatagagacttatatgacaagttgcaatttacaagtcttgaaattccacgtaggaatgtggcaactt
tacaagctgaaaatgtaacaggactctttaaagattgtagtaaggtaatcactgggttacatcctacacaggcacctacacacctcagtgttgac
actaaattcaaaactgaaggtttatgtgttgacatacctggcatacctaaggacatgacctatagaagactcatctctatgatgggttttaaaatga
attatcaagttaatggttaccctaacatgtttatcacccgcgaagaagctataagacatgtacgtgcatggattggcttcgatgtcgaggggtgtc
atgctactagagaagctgttggtaccaatttacctttacagctaggtttttctacaggtgttaacctagttgctgtacctacaggttatgttgatacacct
aataatacagatttttccagagttagtgctaaaccaccgcctggagatcaatttaaacacctcataccacttatgtacaaaggacttccttggaat
gtagtgcgtataaagattgtacaaatgttaagtgacacacttaaaaatctctctgacagagtcgtatttgtcttatgggcacatggctttgagttgac
atctatgaagtattttgtgaaaataggacctgagcgcacctgttgtctatgtgatagacgtgccacatgcttttccactgcttcagacacttatgcctg
ttggcatcattctattggatttgattacgtctataatccgtttatgattgatgttcaacaatggggttttacaggtaacctacaaagcaaccatgatctgt
attgtcaagtccatggtaatgcacatgtagctagttgtgatgcaatcatgactaggtgtctagctgtccacgagtgctttgttaagcgtgttgactgg
actattgaatatcctataattggtgatgaactgaagattaatgcggcttgtagaaaggttcaacacatggttgttaaagctgcattattagcagaca
aattcccagttcttcacgacattggtaaccctaaagctattaagtgtgtacctcaagctgatgtagaatggaagttctatgatgcacagccttgtagt
gacaaagcttataaaatagaagaattattctattcttatgccacacattctgacaaattcacagatggtgtatgcctattttggaattgcaatgtcgat
agatatcctgctaattccattgtttgtagatttgacactagagtgctatctaaccttaacttgcctggttgtgatggtggcagtttgtatgtaaataaaca
tgcattccacacaccagcttttgataaaagtgcttttgttaatttaaaacaattaccatttttctattactctgacagtccatgtgagtctcatggaaaac
aagtagtgtcagatatagattatgtaccactaaagtctgctacgtgtataacacgttgcaatttaggtggtgctgtctgtagacatcatgctaatgag
tacagattgtatctcgatgcttataacatgatgatctcagctggctttagcttgtgggtttacaaacaatttgatacttataacctctggaacacttttac
aagacttcagagtttagaaaatgtggcttttaatgttgtaaataagggacactttgatggacaacagggtgaagtaccagtttctatcattaataac
actgtttacacaaaagttgatggtgttgatgtagaattgtttgaaaataaaacaacattacctgttaatgtagcatttgagctttgggctaagcgcaa
cattaaaccagtaccagaggtgaaaatactcaataatttgggtgtggacattgctgctaatactgtgatctgggactacaaaagagatgctcca
gcacatatatctactattggtgtttgttctatgactgacatagccaagaaaccaactgaaacgatttgtgcaccactcactgtcttttttgatggtaga
gttgatggtcaagtagacttatttagaaatgcccgtaatggtgttcttattacagaaggtagtgttaaaggtttacaaccatctgtaggtcccaaaca
agctagtcttaatggagtcacattaattggagaagccgtaaaaacacagttcaattattataagaaagttgatggtgttgtccaacaattacctga
aacttactttactcagagtagaaatttacaagaatttaaacccaggagtcaaatggaaattgatttcttagaattagctatggatgaattcattgaa
cggtataaattagaaggctatgccttcgaacatatcgtttatggagattttagtcatagtcagttaggtggtttacatctactgattggactagctaaa
cgttttaaggaatcaccttttgaattagaagattttattcctatggacagtacagttaaaaactatttcataacagatgcgcaaacaggttcatctaa
gtgtgtgtgttctgttattgatttattacttgatgattttgttgaaataataaaatcccaagatttatctgtagtttctaaggttgtcaaagtgactattgacta
tacagaaatttcatttatgctttggtgtaaagatggccatgtagaaacattttacccaaaattacaatctagtcaagcgtggcaaccgggtgttgct
atgcctaatctttacaaaatgcaaagaatgctattagaaaagtgtgaccttcaaaattatggtgatagtgcaacattacctaaaggcataatgatg
aatgtcgcaaaatatactcaactgtgtcaatatttaaacacattaacattagctgtaccctataatatgagagttatacattttggtgctggttctgata
aaggagttgcaccaggtacagctgttttaagacagtggttgcctacgggtacgctgcttgtcgattcagatcttaatgactttgtctctgatgcagatt
caactttgattggtgattgtgcaactgtacatacagctaataaatgggatctcattattagtgatatgtacgaccctaagactaaaaatgttacaaa
agaaaatgactctaaagagggttttttcacttacatttgtgggtttatacaacaaaagctagctcttggaggttccgtggctataaagataacagaa
cattcttggaatgctgatctttataagctcatgggacacttcgcatggtggacagcctttgttactaatgtgaatgcgtcatcatctgaagcatttttaat
tggatgtaattatcttggcaaaccacgcgaacaaatagatggttatgtcatgcatgcaaattacatattttggaggaatacaaatccaattcagttg
tcttcctattctttatttgacatgagtaaatttccccttaaattaaggggtactgctgttatgtctttaaaagaaggtcaaatcaatgatatgattttatctct
tcttagtaaaggtagacttataattagagaaaacaacagagttgttatttctagtgatgttcttgttaacaactaaGTTTAAACctggcgcgcc
agaataaacgaacgccaccatggtgagcaagggcgaggagctgttcaccggggtggtgcccatcctggtcgagctggacggcgacgtaa
acggccacaagttcagcgtgtccggcgagggcgagggcgatgccacctacggcaagctgaccctgaagttcatctgcaccaccggcaagc
tgcccgtgccctggcccaccctcgtgaccaccctgacctacggcgtgcagtgcttcagccgctaccccgaccacatgaagcagcacgacttct
tcaagtccgccatgcccgaaggctacgtccaggagcgcaccatcttcttcaaggacgacggcaactacaagacccgcgccgaggtgaagtt
cgagggcgacaccctggtgaaccgcatcgagctgaagggcatcgacttcaaggaggacggcaacatcctggggcacaagctggagtaca
actacaacagccacaacgtctatatcatggccgacaagcagaagaacggcatcaaggtgaacttcaagatccgccacaacatcgaggac
ggcagcgtgcagctcgccgaccactaccagcagaacacccccatcggcgacggccccgtgctgctgcccgacaaccactacctgagcac
ccagtccgccctgagcaaagaccccaacgagaagcgcgatcacatggtcctgctggagttcgtgaccgccgccgggatcactctcggcatg
gacgagctgtacaagt

| Feature list | | | | |
|---|---|---|---|---|
| ATGC | 1 . . . 4 | 4 | == | homology |
| source | 5 . . . 10 | 6 | == | source |
| XbaI restriction site | 5 . . . 10 | 6 | == | misc_feature |
| source | 11 . . . 12 | 2 | == | source |
| CT dinucleotide | 11 . . . 12 | 2 | == | misc_feature |
| source (part) | 13 . . . 20 | 8 | == | source |
| source (part) | 13 . . . 20 | 8 | == | source |
| SbfI restriction site (part) | 13 . . . 20 | 8 | == | misc_feature |
| source | 24 . . . 32 | 9 | == | source |
| RNA | 24 . . . 32 | 9 | == | misc_feature |
| STOP | 24 . . . 26 | 3 | == | misc_feature |
| ORF10 (part) | 33 . . . 149 | 117 | => | gene |
| ORF10 (part) | 33 . . . 149 | 117 | => | CDS |
| stem loop (part) | 84 . . . 119 | 36 | == | stem_loop |
| stem loop (part) | 104 . . . 132 | 29 | == | stem_loop |
| 3′ UTR (part) | 150 . . . 378 | 229 | == | 3′UTR |
| stem loop (part) | 203 . . . 243 | 41 | == | stem_loop |
| source | 379 . . . 386 | 8 | == | source |
| NotI restriction site | 379 . . . 386 | 8 | == | misc_feature |
| From cov2_frag_1_6 | 379 . . . 386 | 8 | == | misc_feature |
| HDV ribozyme | 387 . . . 454 | 68 | => | ncRNA |
| HBA1-3UTR | 455 . . . 476 | 22 | == | misc_feature |
| AmpR promoter (part) | 477 . . . 581 | 105 | == | promoter |
| CTAA | 1017 . . . 1020 | 4 | == | homology |
| G −> C (remove BsaI) (part) | 1298 . . . 1298 | 1 | == | misc_feature |
| origin (part) | 1613 . . . 2201 | 589 | == | rep_origin |
| pBR322 origin, forward primer (part) | 2102 . . . 2121 | 20 | => | primer_bind |
| source | 2222 . . . 2229 | 8 | == | source |
| AsiSI restriction site | 2222 . . . 2229 | 8 | == | misc_feature |
| CMV enhancer | 2367 . . . 2746 | 380 | == | enhancer |
| CMV promoter | 2747 . . . 2950 | 204 | => | promoter |
| chimeric intron | 3086 . . . 3218 | 133 | == | intron |
| T7 promoter | 3263 . . . 3282 | 20 | == | misc_feature |
| promoter (part) | 3264 . . . 3282 | 19 | == | promoter |
| G > A | 3280 . . . 3280 | 1 | == | misc_feature |
| source | 3283 . . . 3290 | 8 | == | source |
| source (part) | 3283 . . . 3290 | 8 | == | source |
| source (part) | 3283 . . . 3290 | 8 | == | source |
| source (part) | 3283 . . . 3290 | 8 | == | source |
| FseI restriction site (part) | 3283 . . . 3290 | 8 | == | misc_feature |
| 5′ UTR (part) | 3291 . . . 3555 | 265 | == | 5′UTR |
| left_flank | 3291 . . . 3291 | 1 | == | misc_feature |
| HBA1-5UTR | 3556 . . . 3586 | 31 | == | misc_feature |
| Kozak sequence | 3587 . . . 3592 | 6 | == | regulatory |
| mature peptide (part) | 3593 . . . 4132 | 540 | == | mat_peptide |
| source | 4133 . . . 4144 | 12 | == | source |
| Finalised sequence | 4133 . . . 4144 | 12 | == | misc_feature |
| mature peptide (part) | 4145 . . . 5062 | 918 | == | mat_peptide |
| TTCT | 5281 . . . 5284 | 4 | == | homology |
| mature peptide (part) | 5933 . . . 6181 | 249 | == | mat_peptide |
| mature peptide (part) | 6182 . . . 6775 | 594 | == | mat_peptide |
| mature peptide (part) | 6776 . . . 7114 | 339 | == | mat_peptide |
| mature peptide (part) | 7115 . . . 7531 | 417 | == | mat_peptide |
| mature peptide (part) | 7532 . . . 7570 | 39 | == | mat_peptide |
| mature peptide (part) | 7532 . . . 7558 | 27 | == | mat_peptide |
| stem loop (part) | 7566 . . . 7593 | 28 | == | stem_loop |
| stem loop (part) | 7578 . . . 7632 | 55 | == | stem_loop |
| T −> C remove XbaI | 8622 . . . 8622 | 1 | == | misc_feature |
| ATGC | 9104 . . . 9107 | 4 | == | homology |
| mature peptide (part) | 10,327 . . . 12,129 | 1803 | == | mat_peptide |

### REFERENCES

1. Dobson A P, Carper E R. Infectious diseases and human population history. Bioscience. 1996; 46(2):115-26.
2. Bengis R, Leighton F, Fischer J, Artois M, Morner T, Tate C. The role of wildlife in emerging and re-emerging zoonoses. Revue scientifique et technique-office international des epizooties. 2004; 23(2):497-512.
3. Wolfe N D, Dunavan C P, Diamond J. Origins of major human infectious diseases. Nature. 2007; 447(7142):279-83.
4. Major Epidemics of the Modern Era: 1899-2022 Council on Foreign Relations [Available from: https://www.cfr.org/timeline/major-epidemics-modern-era.
5. Tenforde M W, Self W H, Adams K, Gaglani M, Ginde A A, McNeal T, et al. Association Between mRNA Vaccination and COVID-19 Hospitalization and Disease Severity. JAMA. 2021; 326(20):2043-54.
6. Coronavirus disease (COVID-19): Vaccines World Health Organization (WHO) 2022 [Available from: https://www.who.int/emergencies/diseases/novel-coronavirus-2019/question-and-answers-hub/q-a-detail/coronavirus-disease-(covid-19)-vaccines?adgroupsurvey={adgroupsurvey}&gclid=CjwKCAjw79iaBhAJEiwAPYwoCKkvwtgJUuC7igm XsySXzZiNvX7y6INegiYnrQKZtyd6TAaRwkw7pxoCkhMQAvD_BwE.
7. Liang Y, Huang L, Liu T. Development and Delivery Systems of mRNA Vaccines. Frontiers in Bioengineering and Biotechnology. 2021; 9.

8 Gu Y, Duan J, Yang N, Yang Y, Zhao X. mRNA vaccines in the prevention and treatment of diseases. MedComm. 2022; 3(3):e167.
9. Alden M, Olofsson Falla F, Yang D, Barghouth M, Luan C, Rasmussen M, et al. Intracellular Reverse Transcription of Pfizer BioNTech COVID-19 mRNA Vaccine BNT 162b2 In Vitro in Human Liver Cell Line. Current Issues in Molecular Biology. 2022; 44(3):1115-26.
10. Tinari S. The EMA covid-19 data leak, and what it tells us about mRNA instability. BMJ. 2021; 372:n627.
11. Pardi N, Hogan M J, Porter F W, Weissman D. mRNA vaccines—a new era in vaccinology. Nature Reviews Drug Discovery. 2018; 17(4):261-79.
12. Brito L A, Kommareddy S, Maione D, Uematsu Y, Giovani C, Berlanda Scorza F, et al. Chapter Seven-Self-Amplifying mRNA Vaccines. In: Huang L, Liu D, Wagner E, editors. Advances in Genetics. 89: Academic Press; 2015. p. 179-233.
13. Tatsis N, Ertl H C. Adenoviruses as vaccine vectors. Mol Ther. 2004; 10(4):616-29.
14. Moss B, Earl P L. Overview of the vaccinia virus expression system. Curr Protoc Mol Biol. 2002; Chapter 16: Unit16.5.
15. Villwock S K, Aparicio O M. Two-Dimensional Agarose Gel Electrophoresis for Analysis of DNA Replication. In: Smith J S, Burke D J, editors. Yeast Genetics: Methods and Protocols. New York, N Y: Springer New York; 2014. p. 329-40.
16. Wang Z, Troilo P J, Wang X, Griffiths T G, Pacchione S J, Barnum A B, et al. Detection of integration of plasmid DNA into host genomic DNA following intramuscular injection and electroporation. Gene Therapy. 2004; 11(8):711-21.
17. Nichols W W, Ledwith B J, Manam S V, Troilo P J. Potential DNA vaccine integration into host cell genome. Ann N Y Acad Sci. 1995; 772:30-9.
18. Stephen S L, Montini E, Sivanandam V G, Al-Dhalimy M, Kestler H A, Finegold M, et al. Chromosomal integration of adenoviral vector DNA in vivo. J Virol. 2010; 84(19):9987-94.
19. Milholland B, Dong X, Zhang L, Hao X, Suh Y, Vijg J. Differences between germline and somatic mutation rates in humans and mice. Nature Communications. 2017; 8(1):15183.
20. Lindsay S J, Rahbari R, Kaplanis J, Keane T, Hurles M E. Similarities and differences in patterns of germline mutation between mice and humans. Nature Communications. 2019; 10(1):4053.
21. Wei J, Matthews P C, Stoesser N, Maddox T, Lorenzi L, Studley R, et al. Anti-spike antibody response to natural SARS-CoV-2 infection in the general population. Nature Communications. 2021; 12(1):6250.
22. Sameer A S, Nissar S. Toll-Like Receptors (TLRs): Structure, Functions, Signaling, and Role of Their Polymorphisms in Colorectal Cancer Susceptibility. Biomed Res Int. 2021; 2021:1157023.
23. Blakney A K, Mckay P F, Yus B I, Aldon Y, Shattock R J. Inside out: optimization of lipid nanoparticle formulations for exterior complexation and in vivo delivery of saRNA. Gene Therapy. 2019; 26(9):363-72.
24. Uddin M N, Roni M A. Challenges of Storage and Stability of mRNA-Based COVID-19 Vaccines. Vaccines. 2021; 9(9):1033.
25. Karikó K, Muramatsu H, Welsh F A, Ludwig J, Kato H, Akira S, et al. Incorporation of pseudouridine into mRNA yields superior nonimmunogenic vector with increased translational capacity and biological stability. Mol Ther. 2008; 16(11):1833-40.
26. Ndeupen S, Qin Z, Jacobsen S, Bouteau A, Estanbouli H, Igyártó B Z. The mRNA-LNP platform's lipid nanoparticle component used in preclinical vaccine studies is highly inflammatory. iScience. 2021; 24(12).
27. Ying B, Whitener B, VanBlargan L A, Hassan A O, Shrihari S, Liang C Y, et al. Protective activity of mRNA vaccines against ancestral and variant SARS-CoV-2 strains. bioRxiv. 2021.
28. Scheaffer S M, Lee D, Whitener B, Ying B, Wu K, Liang C-Y, et al. Bivalent SARS-CoV-2 mRNA vaccines increase breadth of neutralization and protect against the BA.5 Omicron variant in mice. Nature Medicine. 2023; 29(1):247-57.
29. Darling T L, Ying B, Whitener B, VanBlargan L A, Bricker T L, Liang C Y, et al. mRNA-1273 and Ad26.COV2.S vaccines protect against the B.1.621 variant of SARS-CoV-2. Med (N Y). 2022; 3(5):309-24.e6.
30. Tscherne A, Schwarz J H, Rohde C, Kupke A, Kalodimou G, Limpinsel L, et al. Immunogenicity and efficacy of the COVID-19 candidate vector vaccine MVA-SARS-2-S in preclinical vaccination. Proc Natl Acad Sci USA. 2021; 118(28).
31. Gu H, Chen Q, Yang G, He L, Fan H, Deng Y Q, et al. Adaptation of SARS-CoV-2 in BALB/c mice for testing vaccine efficacy. Science. 2020; 369(6511):1603-7.
32. Wu K, Choi A, Koch M, Elbashir S, Ma L, Lee D, et al. Variant SARS-CoV-2 mRNA vaccines confer broad neutralization as primary or booster series in mice. bioRxiv. 2021.
33. Majdoubi A, Michalski C, O'Connell S E, Dada S, Narpala S, Gelinas J, et al. A majority of uninfected adults show preexisting antibody reactivity against SARS-CoV-2. JCI Insight. 2021; 6(8).
34. Yoshioka N, Gros E, Li H R, Kumar S, Deacon D C, Maron C, et al. Efficient generation of human iPSCs by a synthetic self-replicative RNA. Cell Stem Cell. 2013; 13(2):246-54.
35. Jose Russo IHR. Techniques and Methodological Approaches in Breast Cancer Research. 1 ed: Springer New York, N Y; 2014. p. 95.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings. Titles, headings, or the like are provided to enhance the reader's comprehension of this document, and should not be read as limiting the scope of the present invention.

1. A vector comprising:
one or more promoters, wherein said one or more promoters are capable of prokaryotic and eukaryotic expression;
replicon protein genes from Venezuelan equine encephalitis (VEE) virus under the control of the one or more promoters;
a sub-genomic promoter from the VEE virus,
a multi-cloning site for insertion of a nucleic acid encoding a payload under the control of the sub-genomic promoter or a nucleic acid encoding a payload under the control of the sub-genomic promoter and
optionally resistance gene(s) for mammalian and/or bacterial cell culture.

2. The vector of claim 1, wherein said one or more promoters is CMV and T7 promoters or a CMV and T7 binary promoter.

3. The vector of claim 1, wherein the replicon protein genes are NSP1-4 replicon protein genes.

4. The vector of claim 1, wherein the sub-genomic promoter is 26S sub-genomic promoter from the VEE virus.

5. The vector of claim 1, wherein said one or more promoters is a CMV and T7 binary promoter;
wherein the replicon protein genes are NSP1-4 replicon protein genes from Venezuelan equine encephalitis (VEE) virus under the control of the binary promoter; and
wherein the sub-genomic promoter is a 26S sub-genomic promoter from the VEE virus.

6-7. (canceled)

8. A freeze-dried plasmid DNA nanomaterial comprising the vector of of claim 1.

9. A self-amplifying RNA vector produced by transcribing the vector of claim 1 in vitro.

10. A pharmaceutical composition comprising the vector of of claim 1 and a pharmaceutically acceptable carrier.

11. The pharmaceutical composition of claim 10, wherein said vector is not-encapsulated with LNPs

12. The pharmaceutical composition of claim 10, further comprising an adjuvant.

13. A method of delivering a payload of interest to a cell, the method comprising contacting the cell with the vector of claim 1 which expresses the payload.

14. A method of delivering a payload of interest to a cell, the method comprising transcribing the vector of claim 1 which expresses the payload to produce a RNA vector, and contacting the cell with the RNA vector.

15-16. (canceled)

17. A method of treating, and/or preventing disease a subject, said method comprising administering the vector of claim 1, or transcribing in vitro the vector of claim 1 which expresses the payload to produce a RNA vector and administering the RNA vector wherein said vector expresses a therapeutic polypeptide effective against said disease.

18. A method of stimulating an antigen-specific immune response, said method comprising administering the vector of claim 1 or transcribing in vitro the vector of claim 1 to produce a RNA vector and administering the RNA vector, wherein said vector expresses one or more immunogens or epitopes from said infectious agent.


SEQUENCE LISTING (19 sequences)

SEQ ID NO: 1 - DNA, 39 bp, synthetic construct
  source 1..39: mol_type = other DNA
cggtcatggtggcgaataagcggcctttaatgtggaatg

SEQ ID NO: 2 - DNA, 20 bp, synthetic construct
  source 1..20: mol_type = other DNA
caccttgatgccgttcttct

SEQ ID NO: 3 - DNA, 19 bp, synthetic construct
  source 1..19: mol_type = other DNA
cggtcatggtggcgaataa

SEQ ID NO: 4 - DNA, 22 bp, synthetic construct
  source 1..22: mol_type = other DNA
ccgagagctggttaggagatta

SEQ ID NO: 5 - DNA, 21 bp, synthetic construct
  source 1..21: mol_type = other DNA
gcttgtcggccatgatataga

SEQ ID NO: 6 - DNA, 21 bp, synthetic construct
  source 1..21: mol_type = other DNA
gtgcaaggcagtagaatcaag

SEQ ID NO: 7 - DNA, 21 bp, synthetic construct
  source 1..21: mol_type = other DNA
gatgaacttcagggtcagctt

SEQ ID NO: 8 - DNA, 20 bp, synthetic construct
  source 1..20: mol_type = other DNA
gccgtcatctggctcaataa

SEQ ID NO: 9 - DNA, 22 bp, synthetic construct
  source 1..22: mol_type = other DNA
cctgcttctcgtactgctttag

SEQ ID NO: 10 - DNA, 15185 bp, synthetic construct
  source 1..15185: mol_type = other DNA
aacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctgtttaaacagcatatggcgcgcctaaacgaacgccaccatgttcgtatttctcgtcctcctcccacttgtttctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccattgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatgaaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaaacttgattctaaggttggtggtaattataattaccgctatagattgtttaggaagtctaatctcaaaccttttgagagagatatttcaactgaaatctatcaggccggtagcacaccttgtaatggtgttcagggttttaattgttactttcctttacaatcatatggtttccaacccacttatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgatgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctagtgtagctagtcaatccatcattgcctacactatgtcacttggtgcagaaaattcagttgcttactctaataactctattgccatacccattaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatccttgcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacacgaaaagaacttcacaactgctcctgccatttgtcatgatggaaaagcacactttcctcgtgaaggtgtctttgtttcaaatggcacacactggtttgtaacacaaaggaatttttatgaaccacaaatcattactacacacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatctggctgggttttatagctggcctgatcgcaattgtaatggtaactataatgttgtgttgcatgacctcttgctgcagctgtttgaagggatgctgttcttgcgggagttgctgtaaatttgatgaggatgacagcgagccggtgttgaaaggagtgaagcttcattatacttcacgactggaggaagaactgcgccgacgcctgactgaataatctagagtgtttaaaccgacccgggcggccgcaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattggcaagctgcttacatagaactcgcggcgattggcatgccgccttaaaatttttattttattttttcttttcttttccgaatcggattttgtttttaatatttcaaaaaaaaaaaaaaaaaaaaaaaaaacgcgtcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgcgatcgcttaattaacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttaatacgactcactatagggccggccatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgtt

SEQ ID NO: 11 - DNA, 12097 bp, synthetic construct
  source 1..12097: mol_type = other DNA
aacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctgtttaaacagcatatgggcgcgccctcagcatcgattcaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagtctagagtgtttaaaccgacccgggcggccgcaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattggcaagctgcttacatagaactcgcggcgattggcatgccgccttaaaatttttattttattttttcttttcttttccgaatcggattttgtttttaatatttcaaaaaaaaaaaaaaaaaaaaaaaaaacgcgtcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgcgatcgcttaattaacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagtaatacgactcactatagggccggccatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgtt

SEQ ID NO: 12 - DNA, 14963 bp, synthetic construct
  source 1..14963: mol_type = other DNA
aacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctgtttaaacagcatatggcgcgcctaaacgaacgccaccatggttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccattgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatgaaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaaacttgattctaaggttggtggtaattataattaccgctatagattgtttaggaagtctaatctcaaaccttttgagagagatatttcaactgaaatctatcaggccggtagcacaccttgtaatggtgttcagggttttaattgttactttcctttacaatcatatggtttccaacccacttatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgatgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctagtgtagctagtcaatccatcattgcctacactatgtcacttggtgcagaaaattcagttgcttactctaataactctattgccatacccattaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatccttgcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacacgaaaagaacttcacaactgctcctgccatttgtcatgatggaaaagcacactttcctcgtgaaggtgtctttgtttcaaatggcacacactggtttgtaacacaaaggaatttttatgaaccacaaatcattactacacacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatcacgactggaggaagaactgcgccgacgcctgactgaataatctagagtgtttaaaccgacccgggcggccgcaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattggcaagctgcttacatagaactcgcggcgattggcatgccgccttaaaatttttattttattttttcttttcttttccgaatcggattttgtttttaatatttcaaaaaaaaaaaaaaaaaaaaaaaaaacgcgtcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgcgatcgcttaattaacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttaatacgactcactatagggccggccatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgtt

SEQ ID NO: 13 - DNA, 3765 bp, synthetic construct
  source 1..3765: mol_type = other DNA
atgtttgtgttcttggtgttgcttccactggtcagttcccaatgcgttaatctcagaacccgaactcaactcccacccgcatatacaaattccttcaccagaggagtgtactatcctgacaaagtgtttcggtcaagtgtcctccactctactcaggacctctttctgcctttcttttctaacgttacatggtttcatgcaatccatgtgtctgggacaaacggcaccaaacgcttcgacaaccctgtattgccattcaatgatggggtgtactttgcctccattgagaaatccaacatcattcgaggatggattttcgggactactctggactcaaagacacagagcctgctgatcgttaacaacgccacaaacgttgtcatcaaagtgtgcgaattccagttttgcaatgatcccttcctggatgtgtactatcacaagaataacaagtcctggatggagagcggagtctacagcagcgcaaacaactgcaccttcgagtacgtgagtcaaccctttctgatggacctggaagggaaacagggaaacttcaagaacctgagagagtttgtctttaagaacatcgacggctattttaagatctatagtaagcatacgcctatcaacctggtaagggatcttccccagggcttttcagccctggaacctttggttgacttgcctattggtatcaatatcaccagatttcagacccttctggcattgcatcggtcttatcttactccaggtgattcctcctccgggtggactgccggcgccgctgcctactatgtcggctatctgcaaccaagaacgttcctgctcaagtacaacgaaaacggcactattacggatgctgttgattgtgccctggaccctctgtctgagactaaatgcaccctcaagagctttaccgttgagaaggggatttaccaaaccagtaatttccgggtccaacccaccgaaagcattgtgcggttcccaaatatcaccaatctgtgtccctttggcgaagtgttcaatgctacaaggtttgcttctgtgtacgcatggaataggaaacgcatctccaattgtgtcgctgattactccgtgctgtacaattccgcctctttctcaaccttcaagtgttatggcgtttcacctaccaaacttaacgacctgtgcttcactaatgtgtatgccgactcttttgtgatacgaggcgatgaagtgagacagattgcaccagggcagaccggcaaaattgccgactacaactacaagcttccagatgactttaccggatgtgttattgcatggaactcaaacaatctggattccaaggtgggtggcaactataactaccgctatagactgttcaggaaatccaacctgaaaccattcgagcgagatataagcacagaaatctaccaggctggaagtaaaccctgcaacggcgtggaagggttcaactgctacttcccattgcagagttacggattccagcctacaaacggggtgggttaccaaccctatcgtgtcgtagtcctgagttttgagctcctccatgccccagccacagtctgtggccccaagaaaagcaccaatctggtgaagaacaaatgcgtgaactttaactttaacggactcacaggaaccggcgtattgacggagagtaacaagaagttcctgccattccagcagttcggtcgcgatattgccgacactaccgacgctgtccgagatccccagacattggagattcttgatatcacaccctgtagtttcggcggagtgagcgtgattacgcccggaaccaataccagcaatcaggttgccgtcctgtatcagggcgtgaattgcaccgaggtacctgtcgccatccacgctgaccaacttacacccacatggcgagtatattccaccggctccaacgtctttcagacacgtgctggatgtctgatcggtgcagaacacgttaataatagctacgagtgtgatatccccatcggtgctggaatatgcgcctcttatcaaactcaaaccaactctcgtaggcgggcacgtagtgtagcatcccaaagtatcattgcctacacaatgagcctcggtgctgagaattctgtcgcctacagcaacaactccattgctatccctactaacttcacaatcagtgtgacaactgaaattctgcccgtatctatgaccaaaacaagcgttgactgcaccatgtacatctgtggcgattctaccgaatgtagcaatctcctcctgcaatacggatcattctgcactcagctgaatcgtgccctcacaggtattgcagttgagcaggacaagaatacgcaggaagtgtttgcccaggtgaagcaaatctacaaaactccacccataaaagactttggcggattcaatttctcacagatcctgcccgatccctcaaaaccctccaagcgtagctttatcgaggatctgctcttcaacaaggtaaccctcgcagatgccggtttcatcaagcagtatggcgattgtctgggagacatcgccgctcgggacctgatctgtgcacagaagttcaatggactgaccgtgctgcctcccttgctgaccgacgagatgatagcccaatacactagcgccctgctggccggcaccatcacttctgggtggacattcggagctggcgctgcccttcagattccttttgctatgcagatggcctaccgctttaacggcatcggtgtgacacaaaacgttctgtatgaaaaccagaaactcatcgccaaccagttcaacagtgctatcggtaagatacaggatagcctgtcatccactgccagcgcattgggaaagttgcagaatgtagtgaaccagaatgcccaggcacttaacaccctggtgaaacagctctcttcaaattttggtgccatttctagcgtgctgaatgacatactgagccggttggacaaggtggaggctgaagtgcagattgataggctgataactgggcgccttcagtctcttcagacctatgtgacccagcagctcatccgcgctgctgaaattcgcgcatccgctaacctggcagcaaccaaaatgtccgagtgtgtgctgggtcagtctaagagagtggacttttgcgggaaggggtatcacctgatgtcttttcctcagtctgcaccccatggtgtggtctttctgcacgtgacttatgtcccagctcaggaaaagaacttcactacagccccagccatctgccacgatgggaaagcccactttcccagggaaggcgtattcgtgtccaatggtactcattggttcgtcactcagagaaatttctacgagccccagattataaccactgacaatacatttgtatccggcaattgtgatgtggttatcgggattgtgaataatactgtttacgatcctttgcagccagagctggactccttcaaggaggagcttgacaaatattttaagaatcacacatcacctgacgtcgacctcggagatatttcaggaatcaatgcttccgtggtcaatattcagaaggagatagacaggctgaatgaggttgccaagaacctcaacgagtctctgatcgatctgcaggagttgggcaagtacgaacagtatatcaaatggccatggtacatttggcttgggttcattgctgggctgatagctatcgtcatggtgacaattatgttgtgttgcatgacatcctgctgtagttgtctgaagggctgctgctcatgcggcagctgttgctaatgatag

SEQ ID NO: 14 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 15 - DNA, 15193 bp, synthetic construct
  source 1..15193: mol_type = other DNA
aacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctgtttaaacagcatatgggcgcgccctcagcatcgattcaattcgccaccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgttatctctgggaccaatggtactaagaggtttgataaccctgtcctaccatttaatgatggtgtttattttgcttccattgagaagtctaacataataagaggctggatttttggtactactttagattcgaagacccagtccctacttattgttaataacgctactaatgttgttattaaagtctgtgaatttcaattttgtaatgatccatttttggaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattatagtgcgtgagccagaagatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttgatgaagtttttaacgccaccagatttgcatctgtttatgcttggaacaggaagagaatcagcaactgtgttgctgattattctgtcctatataatctcgcaccatttttcacttttaagtgttatggagtgtctcctactaaattaaatgatctctgctttactaatgtctatgcagattcatttgtaattagaggtgatgaagtcagacaaatcgctccagggcaaactggaaatattgctgattataattataaattaccagatgattttacaggctgcgttatagcttggaattctaacaagcttgattctaaggttagtggtaattataattacctgtatagattgtttaggaagtctaatctcaaaccttttgagagagatatttcaactgaaatctatcaggccggtaacaaaccttgtaatggtgttgcaggttttaattgttactttcctttacgatcatatagtttccgacccacttatggtgttggtcaccaaccatacagagtagtagtactttcttttgaacttctacatgcaccagcaactgtttgtggacctaaaaagtctactaatttggttaaaaacaaatgtgtcaatttcaacttcaatggtttaaaaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaatatgtcaacaactcatatgagtgtgacatacccattggtgcaggtatatgcgctagttatcagactcagactaagtctcatgctagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaacgtgctttaactggaatagctgttgaacaagacaaaaacacccaagaagtttttgcacaagtcaaacaaatttacaaaacaccaccaattaaatattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaaaggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccataatgcacaagctttaaacacgcttgttaaacaacttagctccaaatttggtgcaatttcaagtgttttaaatgatatcttttcacgtcttgaccctcctgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagattcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacatcacgactggaggaagaactgcgccgacgcctgactgaataatctagagtgtttaaaccgacccgggcggccgcaactaacttaagctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagaattggcaagctgcttacatagaactcgcggcgattggcatgccgccttaaaatttttattttattttttcttttcttttccgaatcggattttgtttttaatatttcaaaaaaaaaaaaaaaaaaaaaaaaaacgcgtcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgcgatcgcttaattaacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttaatacgactcactatagggccggccatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgtt

SEQ ID NO: 16 - DNA, 11873 bp, synthetic construct
  source 1..11873: mol_type = other DNA
atgctctagactcctgcaggtaagtgtttaaaccgatgaatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaacgcgtggccggcatggtcccagcctcctcgctggcgccggctgggcaacatgcttcggcatggcgaatgggacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgaggggaattaattcttgaagacgaaagggccaggtggcacttttcggggaaatgtgggccggcccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggatctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgcgatcgctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataaggttaattaaatgggcggcgatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtatcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagaaaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggttgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggaaagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagaaacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagactaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtatcgagaaccagcctggtatccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagggacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatataggcggcgcatgagagaagcccagaccaattacctacccaaagcaccatggagaaagttcacgttgacatcgaggaagacagcccattcctcagagctttgcagcggagcttcccgcagtttgaggtagaagccaagcaggtcactgatattgaccttgctaaagccagagcgttttcgcatctggctggcagcggcgcgcccgccacgaacttctctctgttaaagcaagcaggagacgtggaagaaaaccccggtcctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagt

SEQ ID NO: 17 - DNA, 11707 bp, synthetic construct
  source 1..11707: mol_type = other DNA
atgctctagactcctgcaggtaagtgtttaaaccgatgaatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaacgcgtggccggcatggtcccagcctcctcgctggcgccggctgggcaacatgcttcggcatggcgaatgggacaataaagtctgagtgggcggcacgaggggaattaattcttgaagacgaaagggccaggtggcacttttcggggaaatgtgggccggcccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggatctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgcgatcgctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataaggttaattaaatgggcggcgatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtatcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagaaaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggttgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggaaagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagaaacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagactaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtatcgagaaccagcctggtatccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagggacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatactcttctggtccccacagactcagagagaacccaccataggcggcgcatgagagaagcccagaccaattacctacccaaagcaccatggagaaagttcacgttgacatcgaggaagacagcccattcctcagagctttgcagcggagcttcccgcagtttgaggtagaagccaagcaggtcactgatattgaccttgctaaagccagagcgttttcgcatctggctggcagcggcgcgcccgccacgaacttctctctgttaaagcaagcaggagacgtggaagaaaaccccggtcctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagt

SEQ ID NO: 18 - DNA, 16572 bp, synthetic construct
  source 1..16572: mol_type = other DNA
atgctctagactcctgcagggtttaaacgaacatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaagcggccgcggccggcatggtcccagcctcctcgctggcgccggctgggcaacatgcttcggcatggcgaatgggacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgcgatcgctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataaggggccggccattaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaaggccaccatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctccagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagactacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacttatatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaagtttaaacctggcgcgccagaataaacgaacgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagt

SEQ ID NO: 19 - DNA, 16400 bp, synthetic construct
  source 1..16400: mol_type = other DNA
atgctctagactcctgcagggtttaaacgaacatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaagcggccgcggccggcatggtcccagcctcctcgctggcgccggctgggcaacatgcttcggcatggcgaatgggacaataaagtctgagtgggcggcacgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgcgatcgctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataaggggccggccattaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagactcttctggtccccacagactcagagagaagccaccatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctccagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagactacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacttatatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaagtttaaacctggcgcgccagaataaacgaacgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagt